(19) United States
(12) Patent Application Publication
(10) Pub. No.: US 2021/0277102 A1
(43) Pub. Date: Sep. 9, 2021

(54) COMPOSITIONS AND METHODS FOR TREATING A TUMOR SUPPRESSOR DEFICIENT CANCER

(71) Applicant: BETH ISRAEL DEACONESS MEDICAL CENTER, INC., Boston, MA (US)

(72) Inventor: PIER PAOLO PANDOLFI, BOSTON, MA (US)

(73) Assignee: BETH ISRAEL DEACONESS MEDICAL CENTER, INC., BOSTON, MA (US)

(21) Appl. No.: 16/328,631

(22) PCT Filed: Aug. 29, 2017
(86) PCT No.: PCT/US2017/049196

§ 371 Date: Feb. 26, 2019

Related U.S. Application Data
(60) Provisional application No. 62/381,281, filed on Aug. 30, 2016.

(51) Int. Cl. C07K 16/24; A61K 31/713; A61K 31/415; A61K 31/426; G01N 33/574; C12Q 1/6886; A01K 67/027; G01N 33/50; A61K 31/7105

(52) U.S. Cl. CPC C07K 16/24; A61K 31/713; A61K 31/415; A61K 31/426; G01N 33/57434; C12Q 1/6886; A01K 2267/0331; G01N 33/5088; A61K 31/7105; C12Q 2600/158; A01K 2207/12; A01K 2227/105; A01K 67/0271

(57) ABSTRACT

As described below, the present invention features compositions and methods of treating cancers characterized by the loss of Pten, Zbtb7a/Pokemon, p53, Pml and other tumor suppressors by inhibiting the expression or activity of CXCL17; and methods for identifying CXCL17 antagonists using a murine platform.

### CROSS-REFERENCE TO RELATED APPLICATIONS

This application claims the benefit of and priority to U.S. Provisional Patent Application Ser. No. 62/381,281, filed Aug. 30, 2016, which is incorporated herein by reference in its entirety.

### STATEMENT OF RIGHTS TO INVENTIONS MADE UNDER FEDERALLY SPONSORED RESEARCH

This invention was made with government support under Grant No. CA102142 awarded by the National Institutes of Health. The government has certain rights in the invention.

### BACKGROUND OF THE INVENTION

The tumor microenvironment (TME) includes an extracellular matrix, fibroblast, blood vessels and immune cells. It has become increasingly clear that all of these components play an important role in tumor progression and response to therapy. In particular, immune cells in the TME are not of a fixed composition, but rather undergo significant morphological and functional changes during tumor evolution. For example, Gr-1+/CD11b+ cells in the TME are a phenotypically heterogeneous population including myeloid-derived suppressor cells (MDSCs) and neutrophils. While MDSCs disrupt tumor immunosurveillance by interfering with T cell activation, neutrophils have been shown to not only have tumor suppressive functions, but also tumor promoting functions in regulating tumor progression and metastasis. These data suggest that the population of Gr1+/CD11b+ cells in the tumor microenvironment exhibit a high phenotypic heterogeneity, and that their role in tumor progression seems to be strongly context-dependent. Still, the precise tumor characteristics that would trigger a certain phenotype and biological role are not entirely clear.

Although cancer is often associated with chronic inflammation, ‘inflammation-unrelated’ cancers also show significant immune infiltration, suggesting that distinct genetic events in cancer cells could potentially lead to an inflammation-based program that fuels tumor growth. However, it is currently unknown whether, and how, the dynamics of the immune landscape and its evolution are differentially and directly driven by the genetic make-up of cancer, which is in turn limiting the precision of possible therapeutic immune interventions.

Accordingly, compositions and methods for characterizing cancer and providing appropriately tailored therapies are required.

### SUMMARY OF THE INVENTION

As described below, the present invention features compositions and methods of treating cancers characterized by the loss of a tumor suppressor (e.g., Pten, Zbtb7a/Pokemon, p53, Pml) by inhibiting the expression or activity of CXCL17; and methods for identifying CXCL17 antagonists using a murine platform.

In one aspect, the invention features a method of treating a cancer characterized by a deficiency in Pten and p53, the method comprising administering an agent that inhibits the expression or activity of CXCL17 to a subject having a cancer identified as Pten, Zbtb7a/Pokemon, p53, and/or Pml deficient.

In another aspect, the invention features a method of treating a subject having cancer, the method comprising obtaining a biological sample from the subject; detecting a tumor suppressor selected from the group consisting of Pten, Zbtb7a/Pokemon, p53, and Pml in the biological sample, wherein a deficiency in the tumor suppressor indicates the subject could benefit from CXCL17 inhibition; and administering an agent that inhibits CXCL17 expression or activity to the subject, thereby treating the cancer. In one embodiment, the cancer is prostate cancer, breast cancer, colorectal cancer, gastric cancer, pancreatic cancer, or any other cancer of epithelial origin.

In another aspect, the invention features a method of treating prostate cancer in a selected subject, the method comprising administering an agent that inhibits CXCL17 expression or activity to a subject, wherein the subject is selected as having a cancer that is deficient in a tumor suppressor selected from the group consisting of Pten, Zbtb7a/Pokemon, p53, and Pml.

In another aspect, the invention features a mouse comprising a prostate cancer organoid, wherein the organoid expresses endogenous or recombinant CXCL17. In one embodiment, the mouse fails to express or expresses undetectable levels of one or more tumor suppressors selected from the group consisting of Pten, Zbtb7a/Pokemon, p53, and Pml. In another embodiment, the cell is a prostate epithelium cell.

In another aspect, the invention features a method for obtaining an immune-competent murine model for drug screening, the method comprising obtaining one or more neoplastic cells expressing CXCL17 from a mouse having one or more defined genetic lesions; culturing the neoplastic cell in vitro to obtain one or more cancer organoids; and implanting the cancer organoid into a syngeneic mouse not having the defined genetic lesion, thereby obtaining an immune-competent murine model for drug screening.

In another aspect, the invention features a method of identifying an anti-cancer therapeutic agent for a subject having one or more defined genetic lesions, the method comprising obtaining one or more neoplastic cells expressing CXCL17 from a mouse having one or more defined genetic lesions; culturing the neoplastic cell in vitro to obtain one or more cancer organoids; implanting the cancer organoid into an immune competent syngeneic mouse; administering one or more candidate agents to the syngenic mouse; and assaying the biological response of the organoid or syngeneic mouse to the candidate agent. In one embodiment, the defined genetic lesion (e.g., a missense mutation, nonsense mutation, insertion, deletion, or frameshift) is in a tumor suppressor gene selected from the group consisting of Pten, Zbtb7a/Pokemon, p53, and Pml. In another embodiment, the defined genetic lesion results in a loss of expression or function in the tumor suppressor. In another embodiment, the candidate agent is a polypeptide, polynucleotide, or small compound. In another embodiment, the polypeptide is an anti-CXCL17 antibody. In another embodiment, assaying the biological response comprises detecting tumor vascularization, the profile of tumor infiltrating myeloid-derived suppressor cell, chemotaxis of myeloid-derived suppressor cells, correlations of CXCL17 expression levels with changes in Treg numbers, Th1 versus Th2 cytokine profiles, tumor growth, and/or murine survival.

In another aspect, the invention features a method of identifying an anti-cancer therapeutic agent for a subject having one or more defined genetic lesions, the method comprising obtaining one or more neoplastic cells expressing CXCL17 from a set of mice, each having one or more defined genetic lesions; culturing the neoplastic cells in vitro to obtain a set of cancer organoids; implanting each cancer organoid into an immune competent syngeneic mouse; administering one or more candidate agents to the syngenic mouse; and assaying the biological response of the organoid or syngeneic mouse to the candidate agent, wherein a reduction in tumor growth or an increase in mouse survival indicates that the candidate agent is useful for the treatment of a subject having a corresponding defined genetic lesion.

In various embodiments of the above aspects, the agent is an anti-CXCL17 antibody (e.g., a neutralizing antibody). In other embodiments of the above aspects, the agent is an inhibitory nucleic acid molecule (e.g., an antisense molecule, siRNA or shRNA) that inhibits the expression of a CXCL17 protein. In other embodiments, the agent is CID-2745687 or ML-145. In other embodiments of the above aspects, the method inhibits myeloid-derived suppressor cell recruitment, reduces tumor growth, and/or increases subject survival. In other embodiments of the above aspects, the cancer is deficient in Pten and p53; deficient in Pten and Zbtb7a/Pokemon; deficient in Pten, Zbtb7a/Pokemon and p53; or deficient in Pten, p53, Zbtb7a/Pokemon, and Pml)

Compositions and articles defined by the invention were isolated or otherwise manufactured. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Chemokine (C-X-C motif) ligand 17 (CXCL17) polypeptide” is meant a protein or fragment thereof having at least about 85% or greater amino acid sequence identity to NCBI Accession No. Q6UXB2-1 and having CXCL17 biological activity.

sp|Q6UXB2|VCC1_HUMAN VEGF coregulated chemokine 1
OS = Homo sapiens GN = CXCL17 PE = 1 SV = 1
MKVLISSLLLLLPLMLMSMVSSSLNPGVARGHRDRGQASRRWLQEGGQE
CECKDWFLRAPRRKFMTVSGLPKKQCPCDHFKGNVKKTRHQRHHRKPNK
HSRACQQFLKQCQLRSFALPL

By “CXCL17 polynucleotide” is meant a nucleic acid molecule encoding a CXCL17 polypeptide.

By “CXCL17 biological activity” is meant chemokine activity or GPR35 binding activity.

By “CID 2745687” is meant a small compound having the following structure:

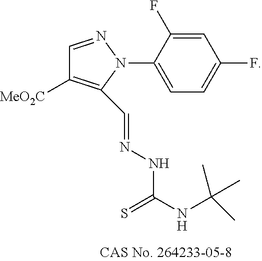 [SMILES: COC(=O)c1cnn(-c2ccc(F)cc2F)c1/C=N/NC(=S)NC(C)(C)C]

CID 2745687 is commercially available, for example, from Tocris.

By “ML 145” is meant a small compound that antagonizes GPR35 and having the following structure:

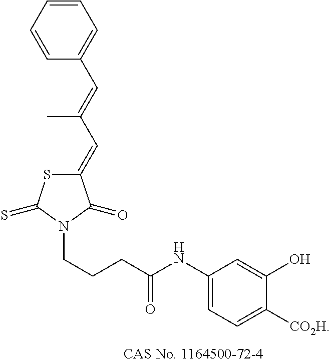 [SMILES: CC(/C=C1\SC(=S)N(CCCC(=O)Nc2ccc(C(=O)O)c(O)c2)C1=O)=C\c1ccccc1]

By “tumor suppressor polypeptide” is meant a protein that represses the development, growth or proliferation of a tumor.

By “tumor suppressor polynucleotide” is meant a polynucleotide encoding a tumor suppressor polypeptide. Exemplary tumor suppressors include Pten, Zbtb7a/Pokemon, p53, and Pml.

By “tumor suppressor deficient” is meant having a reduced level of expression of a tumor suppressor polypeptide or polynucleotide. In one embodiment, the reduction is by at least about 10, 20, 25, 50, or 75% of the level of expression present in a corresponding control cell.

### PTEN

In one embodiment, PTEN expression is undetectable due to a mutation in a polynucleotide encoding PTEN. The sequence of an exemplary Pten polynucleotide is provided below:

| 1 | atgacagcca tcatcaaaga gatcgttagc agaaacaaaa ggagatatca agaggatgga |
|---|---|
| 61 | ttcgacttag acttgaccta tatttatcca aacattattg ctatgggatt tcctgcagaa |
| 121 | agacttgaag gcgtatacag gaacaatatt gatgatgtag taaggttttt ggattcaaag |
| 181 | cataaaaacc attacaagat atacaatctt tgtgctgaaa gacattatga caccgccaaa |
| 241 | tttaattgca gagttgcaca atatcctttt gaagaccata acccaccaca gctagaactt |
| 301 | atcaaaccct tttgtgaaga tcttgaccaa tggctaagtg aagatgacaa tcatgttgca |
| 361 | gcaattcact gtaaagctgg aaagggacga actggtgtaa tgatatgtgc atatttatta |
| 421 | catcggggca aatttttaaa ggcacaagag gccctagatt tctatgggga agtaaggacc |
| 481 | agagacaaaa agggagtaac tattcccagt cagaggcgct atgtgtatta ttatagctac |
| 541 | ctgttaaaga atcatctgga ttatagacca gtggcactgt tgtttcacaa gatgatgttt |
| 601 | gaaactattc caatgttcag tggcggaact tgcaatcctc agtttgtggt ctgccagcta |
| 661 | aaggtgaaga tatattcctc caattcagga cccacacgac gggaagacaa gttcatgtac |
| 721 | tttgagttcc ctcagccgtt acctgtgtgt ggtgatatca aagtagagtt cttccacaaa |
| 781 | cagaacaaga tgctaaaaaa ggacaaaatg tttcactttt gggtaaatac attcttcata |
| 841 | ccaggaccag aggaaacctc agaaaaagta gaaaatggaa gtctatgtga tcaagaaatc |
| 901 | gatagcattt gcagtataga gcgtgcagat aatgacaagg aatatctagt acttacttta |
| 961 | acaaaaaatg atcttgacaa agcaaataaa gacaaagcca accgatactt ttctccaaat |
| 1021 | tttaaggtga agctgtactt cacaaaaaca gtagaggagc cgtcaaatcc agaggctagc |
| 1081 | agttcaactt ctgtaacacc agatgttagt gacaacgaac ctgatcatta tagatattct |
| 1141 | gacaccactg actctgatcc agagaatgaa ccttttgatg aagatcagca tacacaaatt |
| 1201 | acaaaagtc |
| PTEN [Homo sapiens]. | |
| ACCESSION AAD13528 | |
| 1 | mtaiikeivs rnkrryqedg fdldltyiyp niiamgfpae rlegvyrnni ddvvrfldsk |
| 61 | hknhykiynl caerhydtak fncrvaqypf edhnppqlel ikpfcedldq wlseddnhva |
| 121 | aihckagkgr tgvmicayll hrgkflkaqe aldfygevrt rdkkgvtips qrryvyyysy |
| 181 | llknhldyrp vallfhkmmf etipmfsggt cnpqfvvcql kvkiyssnsg ptrredkfmy |
| 241 | fefpqplpvc gdikveffhk qnkmlkkdkm fhfwvntffi pgpeetsekv engslcdqei |
| 301 | dsicsierad ndkeylvltl tkndldkank dkanryfspn fkvklyftkt veepsnpeas |
| 361 | sstsvtpdvs dnepdhyrys dttdsdpene pfdedqhtqi tkv |

### Zbtb7a/Pokemon

In one embodiment, Zbtb7a/Pokemon expression is undetectable due to a mutation in a polynucleotide encoding a Zbtb7a/Pokemon polypeptide. The sequence of an exemplary Zbtb7a/Pokemon polynucleotide is provided below:

| Homo sapiens zinc finger and BTB domain containing 7A (ZBTB7A), | |
|---|---|
| transcript variant 2, mRNA. | |
| NP_001304919 | |
| 1 | actgccgcct cccggcccct cggagggagc cagcccagcc gcagccgccg ccaccgccgc |
| 61 | cgccggggcc gggccccctc gccgctgccc cgggaaggag gtctcggcgc ggaagatggc |
| 121 | cggcggcgtg gacggcccca tcgggatccc gttccccgac cacagcagcg acatcctgag |
| 181 | tgggctgaac gagcagcgga cgcagggcct gctgtgcgac gtggtgatcc tggtggaggg |
| 241 | ccgcgagttc cccacgcacc gctcggtgct ggccgcctgc agccagtact tcaagaagct |
| 301 | gttcacgtcg ggcgccgtgg tggaccagca gaacgtgtac gagatcgact tcgtcagcgc |
| 361 | cgaggcgctc accgcgctca tggacttcgc ctacacggcc acgctcaccg tcagcacagc |
| 421 | caacgtgggt gacatcctca gcgccgcccg cctgctggag atccccgccg tgagccacgt |
| 481 | gtgcgccgac ctcctggacc ggcagatcct ggcggccgac gcgggcgccg acgccgggca |
| 541 | gctggacctt gtagatcaaa ttgatcagcg caacctcctc cgcgccaagg agtacctcga |
| 601 | gttcttccag agcaacccca tgaacagcct gccccccgcg gccgccgccg ccgctgccag |
| 661 | cttcccgtgg tccgcctttg gggcgtccga tgatgacctg gatgccacca aggaggccgt |
| 721 | ggccgccgct gtggccgccg tggccgcggg cgactgcaac ggcttagact tctatgggcc |
| 781 | gggccccccg gccgagcggc ccccgacggg ggacggggac gagggcgaca gcaacccggg |
| 841 | tctgtggcca gagcgggatg aggacgcccc caccgggggt ctctttccgc cgccggtggc |
| 901 | cccgccggcc gccacgcaga acggccacta cggccgcggc ggagaggagg aggccgcctc |
| 961 | gctgtcggag gcggcccccg agccgggcga ctctccgggc ttcctgtcgg gagcggccga |
| 1021 | gggcgaggac ggggacgggc ccgacgtgga cgggctggcg gccagcacgc tgctgcagca |
| 1081 | gatgatgtca tcggtgggcc gggcgggggc cgcggcgggg gacagcgacg aggagtcgcg |
| 1141 | ggccgacgac aagggcgtca tggactacta cctgaagtac ttcagcggcg cccacgacgg |
| 1201 | cgacgtctac ccggcctggt cgcagaaggt ggagaagaag atccgagcca aggccttcca |
| 1261 | gaagtgcccc atctgcgaga aggtcatcca gggcgccggc aagctgccgc gacacatccg |
| 1321 | cacccacacg ggcgagaagc cctacgagtg caacatctgc aaggtccgct tcaccaggca |
| 1381 | ggacaagctg aaggtgcaca tgcggaagca cacgggcgag aagccgtacc tgtgccagca |
| 1441 | gtgcggcgcc gcctttgccc acaactacga cctgaagaac cacatgcgcg tgcacacggg |
| 1501 | cctgcgcccc taccagtgcg acagctgctg caagaccttc gtccgctccg accacctgca |
| 1561 | cagacacctc aagaaagacg gctgcaacgg cgtcccctcg cgccgcggcc gcaagccccg |
| 1621 | cgtccggggc ggggcgcccg accccagccc gggggccacc gcgacccccg gcgcccccgc |
| 1681 | ccagcccagc tcccccgacg cccggcgcaa cggccaggag aagcacttta aggacgagga |
| 1741 | cgaggacgag gacgtggcca gccccgacgg cttgggccgg ttgaatgtag cgggcgccgg |
| 1801 | tggaggaggt gacagcggag gtggccccgg ggccgccacc gacggtaact tcacagccgg |
| 1861 | actcgcctaa aaaccaaaaa gagaaaacag aaacccgaga aagagagaga cagagagaga |
| 1921 | gaaaaaaaat cacccaccac ccccccaaaa acacaaaaaa agaaaatcta tctatataca |
| 1981 | gatatctata tctatatata tatatacaga tatatatata tgacgcgtca cagaatctag |
| 2041 | ggtagcgctt tctcagattt ccctcctttc tgacgttttt ctccctccgc aggggccccg |
| 2101 | gccctccctg gctccccttc cccccaccac cccatcgctg ggtttcgggg cttggtttgg |
| 2161 | ggttttttgt aggacacaag gaatccgaga ccccgcacag ccccctgggc acccggcatg |
| 2221 | gggcctgggg cccgatccga ggccctgggc tggggggagg gtagacgtgg gggcgctggg |
| 2281 | gggggactgg ggtgggcttt taatttcctc ccctcgctgg tttctatgag tctttcagac |
| 2341 | aagaccttaa atgatttctg tctgctctga gcggacgtta aaatgggccc ccgtcccccg |
| 2401 | acccgcaccc tccttcctca gggcacttac taagggaggg gtctccctct ccatctcccc |
| 2461 | agtggcctcc ccgcctccaa ccctgcctgc ggcctccccc cgtcgcccac cccacgtctc |
| 2521 | ctggccactg agacacaaac ctatttattt ctaggcctgg agaaaggaga tcggactggg |
| 2581 | gttcccggtg gggcgccagg atggctcctg ggggtgctcc tgccgccttc cttcacggca |
| 2641 | cttacaaccg gcgggacccc cagggaccac ccctcagggc gcccccccac ccccgcccgg |
| 2701 | tccacctaga cccccacgtt tggagattca aaacttctgt cttcgtcctc tcccccgagc |
| 2761 | cccctctccc aaatttttaa agcacttttt agattttttt ttctctttcc tccttaaaaa |
| 2821 | caaaatttat atatagatat atatatatat ataaataata tacttttcct cagaggagca |
| 2881 | ggcaacagtg tgggataaac agagtcacga tcagaggaac cccagggtct ggtgatggca |
| 2941 | gggatggggg gagagagaga aaatccacaa attccaatgt cacaaaagca ataaaacaaa |
| 3001 | ctagaaaaaa aaaaggtttt acaaaatgaa aggaaggaaa aaaaaaaagg caaccaacca |
| 3061 | cattagaagt cttggcactt tgtaacggaa cgggtactac actttatctt aattcttaat |
| 3121 | ttaaaaacat gtttacaagt tacaaccaac ttctatgaaa agttgaaaag acaaaaaaaa |
| 3181 | aaaaaaaaag cgagcgagag agagagcgag agagagagcg agagcagaag aaattcctaa |
| 3241 | aagtcgattt atttttgtac aaaataataa aaaaaaaaac ccaccacaaa cgtagaatcc |
| 3301 | acttctgttc cccaaaaagc gagaaggggg gttcaggagg aagccatcgc aggggacctg |
| 3361 | ggagacgccc cgaggtgttt gtgcttcacc cccagacgtc agcctcgaag gcaggactgt |
| 3421 | ggggtgttcg tgctgtgttc cccccgctcc ccctttctgt cccctttttt ggttctgacg |
| 3481 | tgaagaggtc ttagcgcccg cttctgtcca cggggtctct ccttcctcct ccctagctca |
| 3541 | gggatgggcc ttccagccgg agcaccccga tccccatccg gcacccccca atcccccaac |
| 3601 | acgcctgtcc ctcccgcatg gccaccaagg agctggacct tggatgcgcc taccctgctg |
| 3661 | aggtgggtga caggggcccc ccacctccag ggccttagaa ccaccgcccc tctccccacc |
| 3721 | ccaggcaccc ctctttttac tcaaaggcac tgactgtaat ccagggggac tgggacctgc |
| 3781 | ctccccccaa cctctggctc ccacaaggcc cggtgttgac cgagccacag gccacggaca |
| 3841 | ggggccgggg ttggggagac tatgtcgcca gatgccagga cgccctcacc ccgtttgcat |
| 3901 | atgcaatgct agcatgggac cccgaaaata gacgctctgc tgcactgaga cttcttgtca |
| 3961 | atgcccaacc ggcggggggg tgtctccctg cccccgaccc ccccataccc ccttctctgt |
| 4021 | gacacacaca tcttctcgtc tctttttctt tcattgttaa agggaagctt tttaagaagg |
| 4081 | caattttcat attgtttcta caggatggtt ttggttccct tcccttccca ccccccctta |
| 4141 | agcctgtcag ccccctccaa atgtctcagg atcccccctc tcccctgggg ctgggtgaca |
| 4201 | gcaccccggc tgcgttcaca ccccagtgtc acagggcgag ctgttctgga gagaaaacca |
| 4261 | tctgtcgtgg ctgagcgggg agcttgaaca cccaggccag ggacacccct ccccagctcc |
| 4321 | cagagaggcc ccctgagggg tgagccctct ttccaccttc ccctatccat gcaccccctc |
| 4381 | gcaataaaac caactctaaa atcacagctg tcgtcctagc cagtgggggc gaccggactt |
| 4441 | ggggggtgga gccctctggg acttccgtag gaacaagggc tgcggcccac cgcgacactt |
| 4501 | acacagacct cggggattgc actaaaccct cgttcctagc tccgcactca gcttcgcctg |
| 4561 | tcctgcccgc ccactttgcc ttaactaccc gcccgtcctg ggggccacag cctctgcatg |
| 4621 | ggcccagagc cgggaccccc ccagcccagc cccgccctcc ccagactccg cgcaatcaca |
| 4681 | tactgtatat agacgtgaat cgattttatt tttattcttt aaattaaggt cgtgataaag |
| 4741 | tgttgccaaa gatacctgct gaattctcgc gtttcaggaa acaaacaaac aaaaaaaaat |
| 4801 | gatatttgag gagggtcgtg ttgactccat atgaaaggac acagctcaaa gcttttttgt |
| 4861 | ttggttgttt ggggtttttt gtgttttctt tttttggggt gttttttttt taactgcctg |
| 4921 | gtacaaaaaa aaaaagagaa aaaaaaaaaa gaaaaacaat gcgaaattgt tatttccatt |
| 4981 | ctcatggtga agttgcgtgg acgcgtgtgt gcgtgtgtgc aagagagcgg gagtgaggtc |
| 5041 | caggctgggg ttggggggct tcaggcgggg gcgcccgggg gccggggagg tggccgggcc |
| 5101 | ggagcccccg tctgcagtgc cccccagcct gccgggccca ggagagagag agaagcatct |
| 5161 | ttgctactag ctgttgctgc tacctgcctc tgccccccga cgccccccgc cttttgagat |
| 5221 | taaggaaaaa aaaaaaaagt caaaaaagtt tttaaaaatg aaaaaaaaaa attataaacc |
| 5281 | agtgaatgta aaatgccgga gcaggcccgg cctggcatgg gtgtggacct gcagccaggc |
| 5341 | aggctcgagc gggcgatacc aaagtctgcc cccccaccat tgtggccatg cagtcctgtc |
| 5401 | actgtctttt tgcttccttc cgaggggggt cccccagcct cttccagggt cttcccctgg |
| 5461 | aagtgggcgg ctgcagggaa ggtgggggac aggggtcttt gcacgattca gaccccgggg |
| 5521 | ccgtggcagg agcggtcacc tcacaggtgg tgacactgag gcaggggcct cggggtgccc |
| 5581 | cctcccgccc ggcaaccaga atggttggag gcaagacaga gagaatgaaa ggaaaaacag |
| 5641 | aagaaaaaaa aatattaaaa accaacaaaa aaagcaaaaa tcctattttt tgagaaagaa |
| 5701 | agatatttat atttgcagtt ttattttaaa aagttattta agttgaagca gccttcctgg |
| 5761 | aggtgggggg gggggggtgg tgggtggctg gcgcaggacg ggtcaggggc ctggaggctg |
| 5821 | ggggtgcccc aggagctaca acctcagagt taagactagc tcgcattaaa tacatagatt |
| 5881 | tacggggggg gggggggggg gccgggccca gggggtggag ggggccaggg agacccccat |
| 5941 | ccctcgccgg ggctgcctgg aggctgtgga ccaggatccg atgcccaggt cccgcccccc |
| 6001 | accccacccc aggcccagaa tcgaggtgcc ttggactttg gaggggccag gcctggtgaa |
| 6061 | tggggggcgg ggcggcgccc tcagggtaca gagcacagac agatagacat tccagagact |
| 6121 | gtattgagag tctttataaa gtgtgggaga tttaaaaaaa aaaaaaactg ataaaaatgc |
| 6181 | actttttggg agtggggagg gagaagcttt aaaagtaata aaaaacaaac aaaaacacaa |
| 6241 | aagatgaaaa aacaaaaaaa ttcatttttc ttgtacataa aaaaaaaaaa agaaccacta |
| 6301 | aacgcagcct gttacgac |

The sequence of an exemplary Zbtb7a/Pokemon protein is provided below:

| Zinc finger and BTB domain-containing protein 7A | |
|---|---|
| NCBI Reference Sequence: NM_001317990.1 | |
| 1 | maggvdgpig ipfpdhssdi lsglneqrtq gllcdvvilv egrefpthrs vlaacsqyfk |
| 61 | klftsgavvd qqnvyeidfv saealtalmd faytatltvs tanvgdilsa arlleipavs |
| 121 | hvcadlldrq ilaadagada gqldlvdqid qrnllrakey leffqsnpmn slppaaaaaa |
| 181 | asfpwsafga sdddldatke avaaavaava agdcngldfy gpgppaerpp tgdgdegdsn |
| 241 | pglwperded aptgglfppp vappaatqng hygrggeeea aslseaapep gdspgflsga |
| 301 | aegedgdgpd vdglaastll qqmmssvgra gaaagdsdee sraddkgvmd yylkyfsgah |
| 361 | dgdvypawsq kvekkiraka fqkcpicekv iqgagklprh irthtgekpy ecnickvrft |
| 421 | rqdklkvhmr khtgekpylc qqcgaafahn ydlknhmrvh tglrpyqcds ccktfvrsdh |
| 481 | lhrhlkkdgc ngvpsrrgrk prvrggapdp spgatatpga paqpsspdar rngqekhfkd |
| 541 | edededvasp dglgrlnvag aggggdsggg pgaatdgnft agla |

### P53

In one embodiment, p53 expression is undetectable due to a mutation in a polynucleotide encoding a p53 polypeptide. The sequence of an exemplary p53 polynucleotide is provided below:

| DEFINITION Homo sapiens mRNA for P53, complete cds. | |
|---|---|
| ACCESSION AB082923 | |
| 1 | cgtgctttcc acgacggtga cacgcttccc tggattggcc agactgcctt ccgggtcact |
| 61 | gccatggagg agccgcagtc agatcctagc gtcgagcccc ctctgagtca ggaaacattt |
| 121 | tcagacctat ggaaactact tcctgaaaac aacgttctgt cccccttgcc gtcccaagca |
| 181 | atggatgatt tgatgctgtc cccggacgat attgaacaat ggttcactga agacccaggt |
| 241 | ccagatgaag ctcccagaat gccagaggct gctccccgcg tggcccctgc accagcagct |
| 301 | cctacaccgg cggcccctgc accagccccc tcctggcccc tgtcatcttc tgtcccttcc |
| 361 | cagaaaacct accagggcag ctacggtttc cgtctgggct tcttgcattc tgggacagcc |
| 421 | aagtctgtga cttgcacgta ctcccctgcc ctcaacaaga tgttttgcca actggccaag |
| 481 | acctgccctg tgcagctgtg ggttgattcc acacccccgc ccggcacccg cgtccgcgcc |
| 541 | atggccatct acaagcagtc acagcacatg acggaggttg tgaggcgctg cccccaccat |
| 601 | gagcgctgct cagatagcga tggtctggcc cctcctcagc atcttatccg agtggaagga |
| 661 | aatttgcgtg tggagtattt ggatgacaga aacacttttc gacatagtgt ggtggtgccc |
| 721 | tatgagccgc ctgaggttgg ctctgactgt accaccatcc actacaacta catgtgtaac |
| 781 | agttcctgca tgggcggcat gaaccggagg cccatcctca ccatcatcac actggaagac |
| 841 | tccagtggta atctactggg acggaacagc tttgaggtgc atgtttgtgc ctgtcctggg |
| 901 | agagaccggc gcacagagga agagaatctc cgcaagaaag gggagcctca ccacgagctg |
| 961 | cccccaggga gcactaagcg agcactgtcc aacaacacca gctcctctcc ccagccaaag |
| 1021 | aagaaaccac tggatggaga atatttcacc cttcagatcc gtgggcgtga gcgcttcgag |
| 1081 | atgttccgag agctgaatga ggccttggaa ctcaaggatg cccaggctgg gaaggagcca |
| 1141 | ggggggagca gggctcactc cagccacctg aagtccaaaa agggtcagtc tacctcccgc |
| 1201 | cataaaaaac tcatgttcaa gacagaaggg cctgactcag actgacattc tccacttctt |
| 1261 | gttccccact gacagcctcc cacccccatc tctccctccc ctgccatttt gggttttggg |
| 1321 | tctttgaacc cttgcttgca ataggtgtgc gtcagaagca cccaggactt ccatttgctt |
| 1381 | tgtcccgggg ctccactgaa caagttggcc tgcactggtg ttttgttgtg gggaggagga |
| 1441 | tggggagtag gacataccag cttagatttt aaggttttta ctgtgaggga tgtttgggag |
| 1501 | atgtaagaaa tgttcttgca gttaagggtt agtttacaat cagccacatt ctaggtaggg |
| 1561 | gcccacttca ccgtactaac cagggaagct gtccctcact gttgaatttt ctctaacttc |
| 1621 | aaggcccata tctgtgaaat gctggcattt gcacctacct cacagagtgc attgtgaggg |
| 1681 | ttaatgaaat aatgtacatc tggccttgaa accacctttt attacatggg gtctagaact |
| 1741 | tgaccccctt gagggtgctt gttccctctc cctgttggtc ggtgggttgg tagtttctac |
| 1801 | agttgggcag ctggttaggt agagggagtt gtcaagtctc tgctggccca gccaaaccct |
| 1861 | gtctgacaac ctcttggtga accttagtac ctaaaaggaa atctcacccc atcccacacc |
| 1921 | ctggaggatt tcatctcttg tatatgatga tctggatcca ccaagacttg ttttatgctc |
| 1981 | agggtcaatt tcttttttct tttttttttt ttttttcttt ttctttgaga ctgggtctcg |
| 2041 | ctttgttgcc caggctggag tggagtggcg tgatcttggc ttactgcagc ctttgcctcc |
| 2101 | ccggctcgag cagtcctgcc tcagcctccg gagtagctgg gaccacaggt tcatgccacc |
| 2161 | atggccagcc aacttttgca tgttttgtag agatggggtc tcacagtgtt gcccaggctg |
| 2221 | gtctcaaact cctgggctca ggcgatccac ctgtctcagc ctcccagagt gctgggatta |
| 2281 | caattgtgag ccaccacgtc cagctggaag ggtcaacatc ttttacattc tgcaagcaca |
| 2341 | tctgcatttt caccccaccc ttcccctcct tctccctttt tatatcccat ttttatatcg |
| 2401 | atctcttatt ttacaataaa actttgctgc caaaaaaaaa aaaaaaaaaa a |

The sequence of an exemplary p53 polypeptide is provided below:

| DEFINITION P53 [Homo sapiens]. | |
|---|---|
| ACCESSION BAC16799 AB082923.1 | |
| 1 | meepqsdpsv epplsqetfs dlwkllpenn vlsplpsqam ddlmlspddi eqwftedpgp |
| 61 | deaprmpeaa prvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evhvcacpgr drrteeenlr kkgephhelp |
| 301 | pgstkralsn ntssspqpkk kpldgeyftl qirgrerfem frelnealel kdaqagkepg |
| 361 | gsrahsshlk skkgqstsrh kklmfktegp dsd |

### Pml

In one embodiment, Pml expression is undetectable due to a mutation in a polynucleotide encoding a Pml polypeptide. The sequence of an exemplary Pml polynucleotide is provided below:

| DEFINITION PML [human, mRNA, 2251 nt]. | |
|---|---|
| ACCESSION S50913 | |
| 1 | gccaactggc tcacgcctcc ccttcagctt ctcttcacgc actccaagat ctaaaccgag |
| 61 | aatcgaaact aagctggggt ccatggagcc tgcacccgcc cgatctccga ggccccagca |
| 121 | ggaccccgcc cggccccagg agcccaccat gcctcccccc gagaccccct ctgaaggccg |
| 181 | ccagcccagc cccagcccca gccctacaga gcgagccccc gcttcggagg aggagttcca |
| 241 | gtttctgcgc tgccagcaat gccaggcgga agccaagtgc ccgaagctgc tgccttgtct |
| 301 | gcacacgctg tgctcaggat gcctggaggc gtcgggcatg cagtgcccca tctgccaggc |
| 361 | gccctggccc ctaggtgcag acacacccgc cctggataac gtctttttcg agagtctgca |
| 421 | gcggcgcctg tcggtgtacc ggcagattgt ggatgcgcag gctgtgtgca cccgctgcaa |
| 481 | agagtcggcc gacttctggt gctttgagtg cgagcagctc ctctgcgcca agtgcttcga |
| 541 | ggcacaccag tggttcctca agcacgaggc ccggccccta gcagagctgc gcaaccagtc |
| 601 | ggtgcgtgag ttcctggacg gcacccgcaa gaccaacaac atcttctgct ccaaccccaa |
| 661 | ccaccgcacc cctacgctga ccagcatcta ctgccgagga tgttccaagc cgctgtgctg |
| 721 | ctcgtgcgcg ctccttgaca gcagccacag tgagctcaag tgcgacatca gcgcagagat |
| 781 | ccagcagcga caggaggagc tggacgccat gacgcaggcg ctgcaggagc aggatagtgc |
| 841 | ctttggcgcg gttcacgcgc agatgcacgc ggccgtcggc cagctgggcc gcgcgcgtgc |
| 901 | cgagaccgag gagctgatcc gcgagcgcgt gcgccaggtg gtagctcacg tgcgggctca |
| 961 | ggagcgcgag ctgctggagg ctgtggacgc gcggtaccag cgcgactacg aggagatggc |
| 1021 | cagtcggctg ggccgcctgg atgctgtgct gcagcgcatc cgcacgggca gcgcgctggt |
| 1081 | gcagaggatg aagtgctacg cctcggacca ggaggtgctg gacatgcacg gtttcctgcg |
| 1141 | ccaggcgctc tgccgcctgc gccaggagga gccccagagc ctgcaagctg ccgtgcgcac |
| 1201 | cgatggcttc gacgagttca aggtgcgcct gcaggacctc agctcttgca tcacccaggg |
| 1261 | gaaagatgca gctgtatcca agaaagccag cccagaggct gccagcactc ccagggaccc |
| 1321 | tattgacgtt gacctgcccg aggaggcaga gagagtgaag gcccaggttc aggccctggg |
| 1381 | gctggctgaa gcccagccta tggctgtggt acagtcagtg cccggggcac accccgtgcc |
| 1441 | agtgtacgcc ttctccatca aaggcccttc ctatggagag gatgtctcca atacaacgac |
| 1501 | agcccagaag aggaagtgca gccagaccca gtgccccagg aaggtcatca agatggagtc |
| 1561 | tgaggagggg aaggaggcaa ggttggctcg gagctccccg gagcagccca ggcccagcac |
| 1621 | ctccaaggca gtctcaccac cccacctgga tggaccgcct agccccagga gccccgtcat |
| 1681 | aggaagtgag gtcttcctgc ccaacagcaa ccacgtggcc agtggcgccg gggaggcaga |
| 1741 | ggaacgcgtt gtggtgatca gcagctcgga agactcagat gccgaaaact cggtctcttc |
| 1801 | cagccctcag tctgaggttc tgtattggaa agtgcatgga gcccatggag accgccgagc |
| 1861 | cacagtcctc gccagcccac tcctcgccag cccactcctc gccagcccac tcctcgccag |
| 1921 | tccagtctct gctgagagca caaggagcct ccagcctgcc ctgtggcaca taccaccccc |
| 1981 | cagcttggcc tccccaccag cccgctgagc aggctgccac ccccgatgct gagcctcaca |
| 2041 | gcgagcctcc tgatcaccag gagcgccctg ccgtccaccg tgggatccgc tacctgttgt |
| 2101 | acagagcaca gagagccatc cgccttcgcc atgccctccg cttgcaccct caattgcatc |
| 2161 | gggcccctat tcggacttgg tctccccatg tggtccaagc cagcactcct gccatcacag |
| 2221 | ggcccctcaa ccatcctgcc aatgcccagg a |

The sequence of an exemplary Pml polypeptide is provided below:

| DEFINITION PML [Homo sapiens]. | |
|---|---|
| ACCESSION AAB19601 | |
| 1 | mepaparspr pqqdparpqe ptmpppetps egrqpspsps pterapasee efqflrcqqc |
| 61 | qaeakcpkll pclhtlcsgc leasgmqcpi cqapwplgad tpaldnvffe slqrrlsvyr |
| 121 | qivdaqavct rckesadfwc feceqllcak cfeahqwflk hearplaelr nqsvrefldg |
| 181 | trktnnifcs npnhrtptlt siycrgcskp lccscallds shselkcdis aeiqqrqeel |
| 241 | damtqalqeq dsafgavhaq mhaavgqlgr araeteelir ervrqvvahv raqerellea |
| 301 | vdaryqrdye emasrlgrld avlqrirtgs alvqrmkcya sdqevldmhg flrqalcrlr |
| 361 | qeepqslqaa vrtdgfdefk vrlqdlssci tqgkdaavsk kaspeaastp rdpidvdlpe |
| 421 | eaervkaqvq alglaeaqpm avvqsvpgah pvpvyafsik gpsygedvsn tttaqkrkcs |
| 481 | qtqcprkvik meseegkear larsspeqpr pstskavspp hldgppsprs pvigsevflp |
| 541 | nsnhvasgag eaeervvvis ssedsdaens vssspqsevl ywkvhgahgd rratvlaspl |
| 601 | laspllaspl laspvsaest rslqpalwhi pppslasppa r |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “defined genetic lesion” is meant an alteration in a polynucleotide sequence relative to a wild-type or reference sequence. Exemplary lesions include, but are not limited to, missense mutations, nonsense mutations, insertions, deletions, or frameshifts.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include any cancer characterized by a deficiency in Pten and p53, including but not limited to prostate cancer, breast cancer, colorectal cancer, gastric cancer, ovarian cancer, pancreatic cancer, or any other cancer of epithelial origin. Examples of diseases include any cancer characterized by a deficiency in Pten, Zbtb7a/Pokemon, p53, Pml and/or other tumor suppressors.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Cancers of the invention are those characterized by a reduction in or the loss of markers Pten and p53.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1F show the genetic make-up of prostate cancer dictates the composition of immune infiltrates in the primary tumor. FIG. 1A: Weight in grams of the prostates (anterior lobe) of controls, Pten^{pc-/-}, Pten^{pc-/-}; Zbtb7a^{pc-/-}, Pten^{pc-/-}; Trp53^{pc-/-} and Pten^{pc-/-}; Pml^{pc-/-} mice at 3 months of age. FIG. 1B: Hematoxylin and eosin staining in the prostate tissues (anterior lobe) of controls, Pten^{pc-/-}, Pten^{pc-/-}; Zbtb7a^{pc-/-}, Pten^{pc-/-}; Trp53^{pc-/-} and Pten^{pc-/-}; Pml^{pc-/-} mice at 3 months of age. Black arrows show invasive sites. Scale bars, 0.1 mm. FIG. 1C: Pie charts show percentage of T cells (CD45+/CD3+), B cells (CD45+/CD19+/B220+), Macrophages (CD45+/CD11b+/F4/80+) and CD45+/Gr-1+/CD11b+ cells in the prostate tissues of control mice and respective prostate tumor models at 3 months of age. ‘Other cells’ contain prostate epithelial cells and the other stromal cells. FIG. 1D: Summarized result of the CD45+/Gr-1+/CD11b+ immune cell population from FIG. 1C. FIG. 1E: Weight in grams of the whole prostates of Pten^{pc-/-}; Zbtb7a^{pc-/-}, Pten^{pc-/-}; Trp53^{pc-/-} and Pten^{pc-/-}; Pml^{pc-/-} mice at 6 months of age. FIG. 1F: Pie charts as in FIG. 1C showing results collected from 6 months old mice. Data are represented as mean±SEM.

FIGS. 2A-2H show the characterization of Gr-1+/CD11b+ cells in Pten^{pc-/-}; Zbtb7a^{pc-/-} and Pten^{pc-/-}; Trp53^{pc-/-} prostate tumors. FIG. 2A: May-Grunwald Giemsa staining of Gr-1+/CD11b+ cells sorted from Pten^{pc-/-}; Zbtb7a^{pc-/-} and Pten^{pc-/-}; Trp53^{pc-/-} prostate tumors (anterior prostate lobes, at 3 months of age). FIG. 2B: Expression analysis of sorted Gr-1+/CD11b+ cells from Pten^{pc-/-} (n=2), Pten^{pc-/-}; Zbtb7a^{pc-/-} (n=3) or Pten^{pc-/-}; Trp53^{pc-/-} (n=3) tumors shows differential expressions of Arginase 1 and inducible nitric oxidase (iNOS). FIG. 2C: is a graph that shows a significant upregulation of S100A9 and IL1b in Gr-1+/CD11b+ cells from Pten^{pc-/-}; Zbtb7a^{pc-/-} tumors, and FIG. 2D is a graph that shows a significant upregulation of IL10 and CD40 in Gr-1+/CD11b+ cells from Pten^{pc-/-}; Trp53^{pc-/-} tumors. FIG. 2E: Characterization of the Gr-1 epitopes, Ly-6G and Ly-6C, in CD11b+ cells by flow cytometry and May-Grunwald Giemsa in Pten^{pc-/-}; Zbtb7a^{pc-/-} and Pten^{pc-/-}; Trp53^{pc-/-} tumors at 3 months of age. FIG. 2F: Quantification of the Ly6G+/Ly6C+ and Ly6G−/Ly6C+ cell populations shows at 3 months of age a significant increase of Ly6G+/Ly6C+ cells in Pten^{pc-/-}; Zbtb7a^{pc-/-} (n=5) compared to Pten^{pc-/-}; Trp53^{pc-/-} mice (n=5) that show mainly Ly6G−/Ly6C+ cells. FIG. 2G: Ly6G+/Ly6C+ and Ly6G−/Ly6C+ analysis in Pten^{pc-/-}; Zbtb7a^{pc-/-} and Pten^{pc-/-}; Trp53^{pc-/-} tumors at 6 months of age. FIG. 2H: Expression analysis by qRT-PCR of sorted Ly6G+/Ly6C+ and Ly6G−/Ly6C+ cells from Pten^{pc-/-}; Trp53^{pc-/-} tumors. Data are represented as mean±SEM.

FIGS. 3A-3H show the differential mechanisms of Gr-1+/CD11b+ cell recruitment in Pten^{pc-/-}; Zbtb7a^{pc-/-} and Pten^{pc-/-}; Trp53^{pc-/-} tumors. FIG. 3A: Expression analysis of chemokines in the prostate tumor tissues (anterior lobes) of Pten^{pc-/-} (n=3), Pten^{pc-/-}; Zbtb7a^{pc-/-} (n=4) and Pten^{pc-/-}; Trp53^{pc-/-} (n=3) mice at 3 months of age by qRT-PCR. FIG. 3B: CXCL5 protein expression level in Pten^{pc-/-}, Pten^{pc-/-}; Zbtb7a^{pc-/-} and Pten^{pc-/-}; Trp53^{pc-/-} mice (n=3) at 3 months of age shows that CXCL5 is upregulated in Pten^{pc-/-}; Zbtb7a^{pc-/-} prostate tumors. FIG. 3C: Chromatin immunoprecipitation (ChIP) analysis in RWPE-1 human prostate epithelial cells shows enrichment of CXCL5 locus in Zbtb7a immunoprecipitates, Mia and H19 serve as positive controls. FIG. 3D: Zbtb7a overexpression in RWPE-1 cells leads to a decrease of CXCL5 mRNA levels. FIG. 3E: Sox9 knockdown leads to a decrease of CXCL5 mRNA levels and Zbtb7a knockdown leads to an increase of CXCL5 mRNA levels in RWPE-1 cells. FIG. 3F: ChIP analysis in RWPE-1 cells shows enrichment of CXCL5 locus in Sox9 immunoprecipitates. FIG. 3G: p53 knockdown in RWPE-1 cells leads to an increase of CXCL17 mRNA levels. p21 serves as a positive control. FIG. 3H: ChIP analysis in RWPE-1 cells shows enrichment of CXCL17 locus in p53 immunoprecipitates, p21 serves as positive controls. Data of in vitro cell line experiments are represented as mean of 3 independent biological replicates±SEM.

FIGS. 4A-4K show that CXCL5 and CXCL17 are chemoattractant for polymorphonuclear leukocytes (PMN) cells and monocytes respectively. FIG. 4A: Ly6G+/Ly6C+ and Ly6G−/Ly6C+ flow analysis of Gr1+ cells culture for 4 days in GM-CSF, IL-6 supplemented medium plus either recombinant CXCL5 or recombinant CXCL17 did not show significant changes. FIG. 4B: Transwell migration assay of Gr1+ cells, and (FIG. 4C) monocytes isolated form the bone marrow of healthy mice shows differential migration toward medium supplemented with increasing concentration of either recombinant CXCL5 or CXCL17 (n=3). FIG. 4D: Western blot analysis confirms the specific deletion of the tumor suppressor genes Zbtb7a, PTEN and Trp53 in organoids isolated from our prostate cancer mouse models. FIG. 4E: Haemotoxylin and Eosin (H&E) and immunohistochemistry (IHC) staining showing similar phospho-AKT and Ki67 staining in organoid generated from the prostates of 3 months old Pten^{pc-/-}; Zbtb7a^{pc-/-} and Pten^{pc-/-}; Trp53^{pc-/-} mice. FIG. 4F: CXCL17 qRT-PCR expression analysis in organoids generated from the prostates of wild type, Pten^{pc-/-}; Zbtb7a^{pc-/-} and Pten^{pc-/-}; Trp53^{pc-/-} mice. FIG. 4G: Schematic representation of the experimental strategy used to perform transwell migration assays using organoid conditioned medium. FIG. 4H: Transwell migration assay of monocytes isolated from healthy mice shows increased migration toward conditioned medium from Pten^{pc-/-}; Trp53a^{pc-/-} organoids (n=3). FIG. 4I: CXCL17 qRT-PCR expression analysis in Pten^{pc-/-}; Trp53^{pc-/-} organoids shows the efficacy of the CXCL17 shRNA-mediated knockdown. FIG. 4J: Reduced migration of monocytes, but not of Gr1+ cells (FIG. 4K) in a transwell migration assay performed using conditioned medium from Pten^{pc-/-}; Trp53^{pc-/-} organoids expressing either scramble shRNA or a CXCL17 shRNA (n=3). Data are represented as mean±SEM.

FIGS. 5A-5H show Gr-1+/CD11b+ cells in Pten^{pc-/-}; Zbtb7a^{pc-/-} and Pten^{pc-/-}; Trp53^{pc-/-} prostate tumors promote tumor growth. FIG. 5A: Flow cytometry analysis of CD4+/Foxp3+ cells in the prostate tumors of Pten^{pc-/-}; Zbtb7a^{pc-/-} and Pten^{pc-/-}; Trp53^{pc-/-} at 3 months of age (n=3). FIG. 5B: Purified CD4+ T cells were co-cultured with Gr-1+/CD11b+ cells from Pten^{pc-/-}; Zbtb7a^{pc-/-} and Pten^{pc-/-}; Trp53a^{pc-/-} tumors (n=3) for 3 days followed by flow cytometry to assess the presence of CD4+/Foxp3+ Treg cells. FIG. 5C: Left panel: measurement of tumor growth by MRI in Pten^{pc-/-}; Zbtb7a^{pc-/-} mice treated with control IgG (n=3) or anti-CXCL5 antibody (n=3). Right panel: flow cytometry analysis of Pten^{pc-/-}; Zbtb7a^{pc-/-} prostate tumors after treatment with the anti-CXCL5 antibody shows less Gr-1+/CD11b+ granulocytes (n=3). FIG. 5D: Flow cytometry analysis of CD45+/CD8+ T cells (left) and CD45+/CD4+/FoxP3+ Treg cells (right) in Pten^{pc-/-}; Zbtb7a^{pc-/-} prostate tumors after treatment with the anti-CXCL5 (n=3). FIG. 5E: Left panel: measurement of tumor growth by MRI in Pten^{pc-/-}; Trp53^{pc-/-} mice treated with control IgG (n=3) or anti-Gr1 antibody (n=3). Right panel: flow cytometry analysis of Pten^{pc-/-}; Trp53^{pc-/-} prostate tumors after treatment with the anti-Gr1 antibody shows less Gr-1+/CD11b+ granulocytes (n=3). FIG. 5F: Flow cytometry analysis of CD45+/CD8+ T cells (left) and CD45+/CD4+/FoxP3+ Treg cells (right) in Pten^{pc-/-}; Trp53^{pc-/-} prostate tumors after treatment with the anti-Gr1 antibody (n=3). FIG. 5G: Flow cytometry analysis of Pten^{pc-/-}; Zbtb7a^{pc-/-}, Pten^{pc-/-}; Trp53^{pc-/-} and Pten^{pc-/-}; Pml^{pc-/-} prostate tumors after treatment with the CXCR2 antagonist SB225002 (CXCR2i). FIG. 5H: Left panel: Tumor growth in vehicle (n=3) or CXCR2 antagonist SB225002 (CXCR2i) (n=3) treated mice of Pten^{pc-/-}; Zbtb7apc−/and Pten^{pc-/-}; Trp53^{pc-/-} shows a significant inhibition of tumor growth in both models, whereas Pten^{pc-/-}; Pml^{pc-/-} mice did not show any significant response (n=2). Right panel: representative MRIs of prostate cancers in vehicle or SB225002 (CXCR2i) treated mice of Pten^{pc-/-}; Zbtb7a^{pc-/-} at day 0 (Baseline) and 3 weeks on treatment, Pten^{pc-/-}; Trp53^{pc-/-} at day 0 (Baseline) and 2 weeks on treatment and Pten^{pc-/-}; Pml^{pc-/-} at day 0 (Baseline) and 2 weeks on treatment. Tumor volumes (area outlined by dotted circle) were quantified by using ImageJ software. An asterisk represents the location of the bladder. All data are represented as mean±SEM.

FIGS. 6A-6H show the clinical relevance of the genotype-chemokines-immune phenotype axis of prostate tumor models. FIG. 6A: Left panel: Heat map of the TGCA provisional prostate adenocarcinoma dataset (499 samples) clustered into PMN-high, PMN-middle and PMN-low groups using a gene signature for polymorphonuclear leukocytes myeloid derived suppressor cells (PMN-MDSCs). Right panel: CXCL5 is significantly more expressed in the group of samples that showed higher expression of the PMN-signature. FIG. 6B: Top panel: Heat map of the TGCA provisional prostate adenocarcinoma dataset (499 samples) clustered into Mo-high, Mo-middle and Mo-low groups using a gene signature for monocytic MDSCs/M2 macrophages. Bottom panel: CXCL17 is significantly more expressed in the group of samples that showed higher expression of the Mo-signature. FIG. 6C: Expression level of CXCL5 and CXCL17 in samples of the Robinson dataset (metastatic prostate cancer, n=150) grouped by the status of PTEN (not altered/altered) and the expression level of Zbtb7a (low/high). FIG. 6D: Expression level of CXCL17 and CXCL5 in samples of the Robinson dataset grouped by the status of PTEN and p53 (not altered/altered). FIG. 6E: Clustering of the Robinson into the 3 groups PMN-high, PMN-mid and PMN-low (upper panel), and into the 3 groups T cell-high, T cell-mid and T cell-low (lower panel). FIG. 6F: Distribution of patients with the indicated status of PTEN, p53, Zbtb7a and PML in the different clusters generated by the PMN- and the T-cell-signature. FIG. 6G: PML expression level is significantly lower in the patients categorized in the PMN-low group and in the T cell-low group when compared to the respective high-signature group. FIG. 6H: Immune phenotype model for tumor progression by Gr-1+/CD11b+ cells in Pten^{pc-/-}; Zbtb7a^{pc-/-} and Pten^{pc-/-}; Trp53^{pc-/-} mice.

FIGS. 7A-7D show infiltration of the immune cells in spleen and the prostate tissue of respective mouse models at 3 months of age. FIG. 7A: Percentage of Gr-1+/CD11b+ cells, T cells (CD3+), B cells (CD19+/B220+) and macrophages (CD11b+/F4/80+) in spleen of control mice and respective prostate tumor models at 3 months of age (n≥3). FIG. 7B: Percentage of T cells (CD3+), B cells (CD19+/B220+) and macrophages (CD11b+/F4/80+) in the tumor of control mice and respective prostate tumor models at 3 months of age (n≥3). Data are represented as mean±SEM. FIG. 7C: Gating strategy used for our immune landscape analysis. FIG. 7D: Gating strategy for Gr-1+/CD11b+ cells. Representative flow cytometry blots of Gr-1+/CD11b+ cells in the prostate Pten^{pc-/-}; Zbtb7a^{pc-/-}, Pten^{pc-/-}; Trp53^{pc-/-} and Pten^{pc-/-}; Pml^{pc-/-} mice at 3 months of age.

FIGS. 8A-8F show infiltration of the immune cells in spleen and the prostate tissue of respective mouse models at 3 months of age. FIG. 8A: Percentage of Gr-1+/CD11b+ cells, T cells (CD3+), B cells (CD19+/B220+) and macrophages (CD11b+/F4/80+) in spleen of prostate tumor models at 6 months of age (n=3). FIG. 8B: Percentage of Gr-1+/CD11b+ cells, T cells (CD3+), B cells (CD19+/B220+) and macrophages (CD11b+/F4/80+) in the tumor of prostate cancer models at 6 months of age (n≥3). Data are represented as mean f SEM. FIG. 8C, FIG. 8D, FIG. 8E, and FIG. 8F are representative flow cytometry blots (upper panel) and quantification of the indicated cell populations (lower panel) isolated from the prostate tumor of 6 months old Pten^{pc-/-}; Trp53^{pc-/-} mice (n=3).

FIGS. 9A-9C show localization of immune cells in prostate tumor tissues. FIG. 9A: IHC of the Ly6G epitope in Pten^{pc-/-}; Zbtb7a^{pc-/-} and Pten^{pc-/-}; Trp53^{pc-/-} prostate tumors (anterior prostate lobes, at 3 month of age) shows that Ly6G+ cells are mainly localized in the lumen of prostate glands and are in close proximity to cancer cells (black arrows). Scale bars, 0.05 mm. FIG. 9B: IHC of the CD45R (B220) and CD3 epitope in Pten^{pc-/-}; Zbtb7a^{pc-/-} and Pten^{pc-/-}; Trp53^{pc-/-} prostate tumors at 3 months of age (anterior prostate lobes) shows that B cells and T cells are mainly localized in the stroma of prostate tumor tissue. Scale bars, 0.05 mm. FIG. 9C: Gating strategy for positivity of the Ly6G and Ly6C epitopes.

FIGS. 10A-10C provides graphs of Gr-1+/CD11b+ cells showing a differential tumor promotive activity in Pten^{pc-/-}; Zbtb7a^{pc-/-} and Pten^{pc-/-}; Trp53^{pc-/-} tumors. FIG. 10A: Expression analysis of sorted Gr-1+/CD11b+ cells from Pten^{pc-/-} (n=2), Pten^{pc-/-}; Zbtb7a^{pc-/-} (n=3) and Pten^{pc-/-}; Trp53^{pc-/-} (n=3) tumors shows a specific upregulation of S100A8 in granulocytes from Pten^{pc-/-}; Zbtb7a^{pc-/-} tumors. Data are represented as mean±SEM. FIG. 10B: Expression analysis of sorted Gr-1+/CD11b+ cells from peripheral blood (blood) (n=4) or Pten^{pc-/-}; Zbtb7a^{pc-/-} tumors (n=3) shows increase in expression of S100A9, S100A8 and IL1b in granulocytes from the primary tumor site. FIG. 10C: Expression analysis of sorted CD11b+/Gr1+ cells and tumor cells (CD45−/CD49f+) from Pten^{pc-/-}; Zbtb7a^{pc-/-} (n=3) tumors shows specific expressions of S100A9, IL1b and S100A8 in Gr-1+/CD11b+ cells. Data are represented as mean±SEM.

FIGS. 11A-11D show CXCL5 expression is upregulated in Pten^{pc-/-}; Zbtb7a^{pc-/-} tumors. FIG. 11A: Expression analysis of chemokines from the CXC and CC family using microarray data obtained from prostate tumors (anterior lobes) from 3 month old Pten^{pc-/-} and Pten^{pc-/-}; Zbtb7a^{pc-/-} mice. FIG. 11B: Gene rank list of upregulated genes in Pten^{pc-/-}; Zbtb7a^{pc-/-} vs Pten^{pc-/-} mice at 3 months measured by microarray. FIG. 11C: Expression analysis of sorted CD11b+/Gr1+ cells and tumor cells (CD45−/CD49f+) from Pten^{pc-/-}; Zbtb7a^{pc-/-} (n=3) tumors shows specific expressions of CXCL5 in tumor cells. FIG. 11D: Expression analysis of CXCL5 in the prostate tissues of control (n=3), Zbtb7a^{pc-/-} (n=3) mice and in prostate tumor tissue (anterior lobes) from Pten^{pc-/-}; Zbtb7a^{pc-/-} (n=3) mice at 3 months of age by qRT-PCR.

FIG. 12A provides a graph that shows Ly6G+/Ly6C+ and Ly6G−/Ly6C+ flow analysis of BM cells culture for 4 days in GM-CSF, IL-6 supplemented medium plus either recombinant CXCL5 or recombinant CXCL17.

FIG. 12B provides two graphs that show qRT-PCR gene expression analysis of BM and Gr1+ cells from the experiment in FIG. 12A and the experiment in FIG. 4A. Data are represented as mean±SEM.

FIG. 12C shows representative flow cytometry blots of Gr1+ cells and monocytes isolated from the bone marrow of healthy mice.

FIGS. 13A-13C show that depletion of Gr-1+/CD11b+ cells decreases tumor burden in Pten^{pc-/-}; Zbtb7a^{pc-/-} and Pten^{pc-/-}; Trp53a^{pc-/-} mice. FIG. 13A: Pten^{pc-/-}; Zbtb7a^{pc-/-} mice (4 months of age) were treated with Ly6G-depletion antibody or control IgG antibody every other day for 10 days by intraperitoneal injection (300 ug/mouse) and tumor tissue was subjected to histological analysis. Black arrows show regions of reduced tumor burden. Scale bars, 0.02 mm. FIG. 13B: Histological analysis of Pten^{pc-/-}; Zbtb7a^{pc-/-} and Pten^{pc-/-}; Trp53^{pc-/-} tumors (anterior prostate lobes) treated with Vehicle or SB225002 (CXCR2i) shows reduced tumor burden after CXCR2 inhibition (black arrows). Scale bars, 0.02 mm. FIG. 13C: Flow cytometry analysis of Pten^{pc-/-}; Trp53^{pc-/-} prostate tumors after treatment with SB225002 (CXCR2i) (n=5) and vehicle (n=5) every day for 10 days by intraperitoneal injection shows less Foxp3+ cells. All data are represented as mean±SEM.

FIGS. 14A-14D show that the NFκB pathway is markedly activated through Gr-1+/CD11b+ cells in Pten^{pc-/-}; Zbtb7a^{pc-/-} tumors. FIG. 14A: Gene Set Enrichment Analysis for NFκB targets using microarray data obtained from tumors derived from 3 month old Pten^{pc-/-} and Pten^{pc-/-}; Zbtb7a^{pc-/-} mice. FIG. 14B: Protein level of pIRAK4 (normalized with total IRAK4) and IκBα (normalized with β-actin) in the prostate tumors of 3 month old Pten^{pc-/-}, Pten^{pc-/-}; Zbtb7a^{pc-/-}, and Pten^{pc-/-}; Trp53^{pc-/-} mice (n=3). FIG. 14C: Protein level of IκBα (normalized with β-actin) in the prostate tumors treated with vehicle (n=2) or SB225002 (CXCR2i) (n=3) in Pten^{pc-/-}; Zbtb7a^{pc-/-} mice. FIG. 14D: Expression of CXCL5 in the prostate tumors treated with vehicle (n=3) or SB225002 (CXCR2i) (n=4) in Pten^{pc-/-}; Zbtb7a^{pc-/-} mice.

FIGS. 15A-15B show upregulation of phospho-ERK and β-Catenin in Pten^{pc-/-}; Pmi^{pc-/-} mice. FIG. 15A: IHC of phospho-ERK and β-catenin in Pten^{pc-/-} and Pten^{pc-/-}; Pml^{pc-/-} prostate tumors at 3 months of age (anterior prostate lobes). Scale bars, 0.1 mm. FIG. 15B: Schematic representation of the three different immune landscapes observed in the Pten^{pc-/-}; Zbtb7a^{pc-/-}, Pten^{pc-/-}; Trp53^{pc-/-} and Pten^{pc-/-}; Pml^{pc-/-} mice.

FIG. 16 shows the genetic background of the Control, Pten^{pc-/-}, Pten^{pc-/-}; Zbtb7a^{pc-/-}, Pten^{pc-/-}; Trp53^{pc-/-} and Pten^{pc-/-}; Pml^{pc-/-} experimental mice.

### DETAILED DESCRIPTION OF THE INVENTION

The invention provides compositions and methods featuring agents that inhibit the activity or expression of CXCL17, and use of such agents to treat prostate cancer, breast cancer, and other cancers characterized by the loss of Pten, Zbtb7a/Pokemon, p53, Pml and other tumor suppressors by inhibiting the expression or activity of CXCL17; and methods for identifying CXCL17 antagonists using a murine platform.

The invention is based, at least in part, on the discovery that loss of p53 leads to increased expression of the Gr-1+/CD11b cell attractant, CXCL17. Different genetic backgrounds in prostate cancer were found to affect the composition of the tumor microenvironment. These changes were characterized using a comprehensive co-clinical platform to systematically analyze the immune cell component of faithfully genetically engineered mouse models (GEMMs) of prostate cancer driven only by the loss of the Pten, or the compound loss of Pten along with the Zbtb7a/Pokemon, p53, Pml and other tumor suppressors. This analysis revealed a striking quantitative and qualitative heterogeneity in the infiltration and tumor-promotive function of Gr-1+/CD11b+ cells based on the genetic make-up of the primary tumor.

In Pten^{pc-/-}; Zbtb7a^{pc-/-} tumors, infiltrating Gr-1+/CD11b+ cells exhibited a neutrophilic phenotype that directly promoted tumor progression by impacting the NFκB signaling pathway through the non-cell autonomous secretion of S100A9 and IL1β. In contrast, S100A9 expression and subsequent NFκB signaling activation was not upregulated in Pten^{pc-/-}; p53^{pc-/-} tumors that mainly recruit granulocytic myeloid-derived suppressor cells (MDSCs). Accordingly, the tumor promoting impact of Gr-1+/CD11b+ cells in this model was based on a Treg mediated anti-tumor immune suppression. Human prostate and breast cancer specimens' deficient of p53 showed a significantly higher expression of CXCL17. Loss of Zbtb7a or p53 results in the overexpression of CXCL17 and the consequent aberrant recruitment of tumor promoting granulocytes. Accordingly, the invention provides compositions and methods that reduce the expression or activity of CXCL17. In one embodiment, the invention provides method of treating a cancer characterized by a loss of Zbtb7a or p53 by administering to the subject an effective amount of an anti-CXCL17 antibody.

### Antibodies Against CXCL17 in Human Cancer

Increased expression of CXCL17 mRNA was found in human prostate and breast cancer samples that were PTEN and p53 deficient. Based on these findings, it is likely that CXCL17 functions in other cancer types, including colorectal cancer. CXCL17 is important for recruitment of MDSCs and tumor progression in the Pten^{pc-/-}; Trp53^{pc-/-} model and therefore, a therapeutic antibody specifically neutralizing CXCL17 is likely to inhibit MDSC accumulation, limit tumor growth, and lead to increased survival. Accordingly, the invention provides a therapeutic antibody that accesses CXCL17 within the tumor stroma to bind and neutralize it there. In one embodiment, the neutralizing antibody disrupts CXCL17 binding to its receptor, GPR35.

Antibodies are made by any methods known in the art utilizing a CXCL17 polypeptide, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide of the invention or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

In one embodiment, the invention provides a human-rodent cross reactive CXCL17 neutralizing antibody. In one embodiment, antibodies against the CXCL17 polypeptide are derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

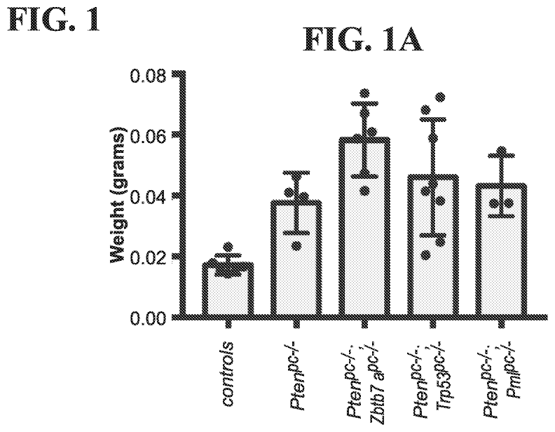

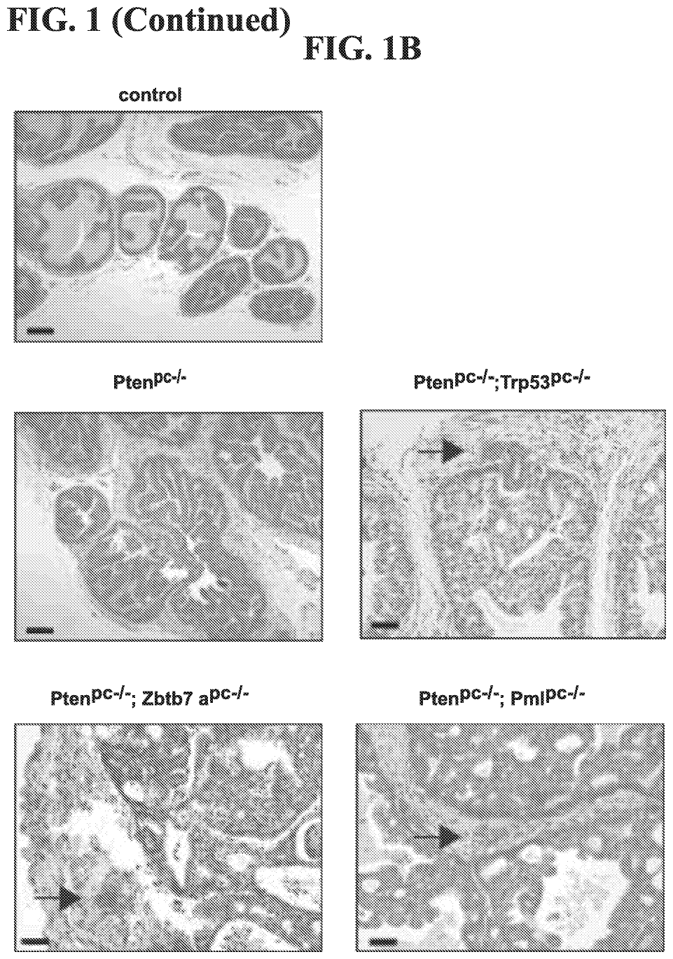

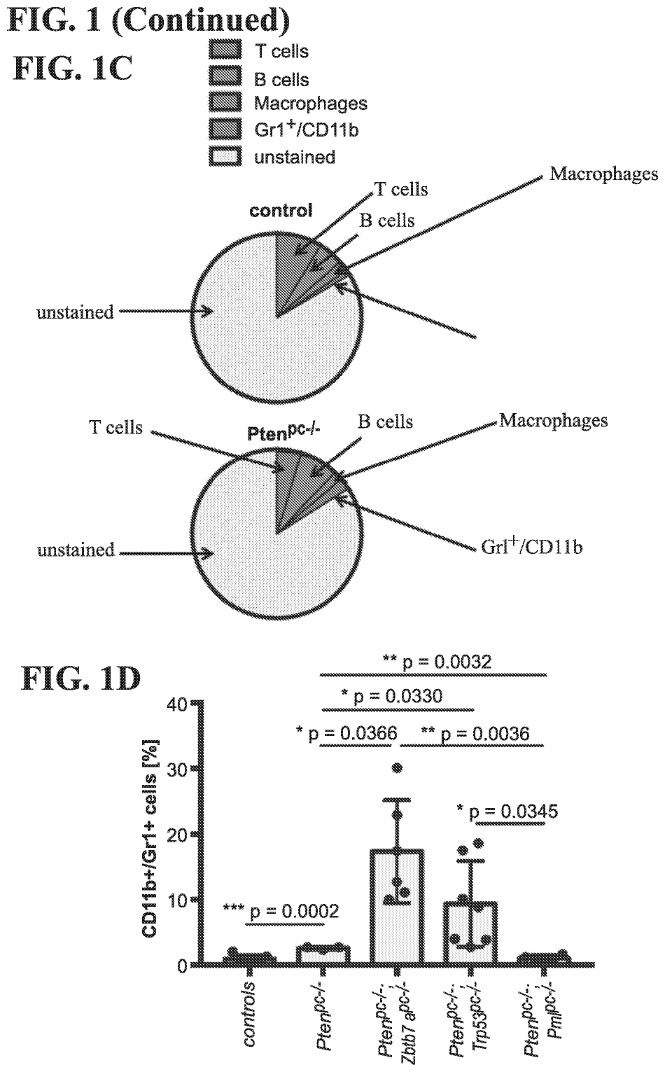

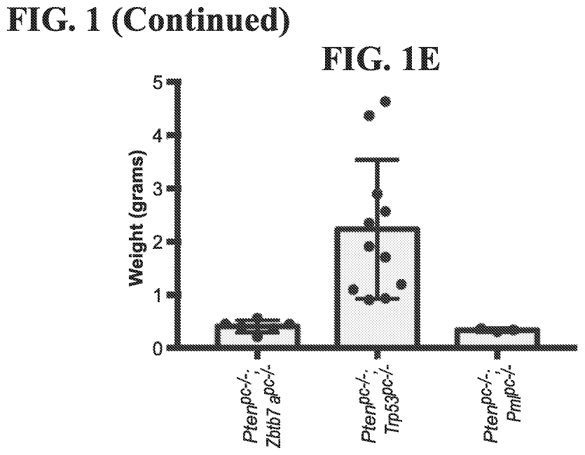

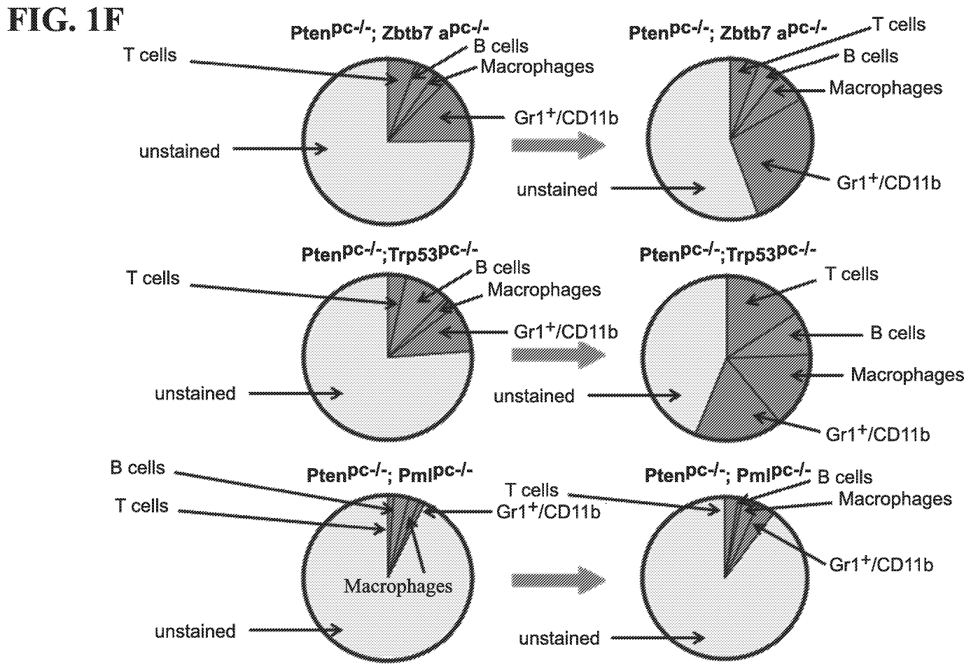

In one embodiment, human phage display scFv libraries are screened for antibodies having the desired properties. A scheme for selecting with human and mouse antigens to drive selection of cross-reactive antibodies is provided at FIG. 1O. In one embodiment, a mouse antigen-only selection is carried out. Such a mouse-specific antibody may be used as a surrogate for a human-specific antibody. Given the high degree of homology between human and cynomolgus monkey CXCL17, antibodies that bind human CXCL17 are likely to also bind the cynomolgus ortholog FIG. 1P). However, to ensure human/cyno cross-reactivity a separate selection branch in which human and cyno proteins are alternately used for selection will be conducted (FIG. 1O).

The general format for phage screening makes use of biotinylated antigens and pull-down strategies. In one embodiment, a CXCL17 protein is chemically biotinylated. In another embodiment, a CXCL17 protein comprises a biotin modification sites (Avi-tag). Once phage binders are isolated, purified or peri-prepped scFVs are evaluated using additional binding and functional assays (e.g., ELISA and Octet biolayer interferometry binding formats (direct binding assays and ligand competition assays). To identify neutralizing antibodies, functional neutralization assays and cell-based ligand binding competition assays are used. Multiple functional assays for CXCL17 and activation of its receptor, GPR35, have been published (Pisabarro et al., The Journal of Immunology; 176(4): 2069-2073, 2006; Wang et al., The Journal of Biological Chemistry; 281(31) 22021-22028, 2006; Matsui et al., PLoS ONE; 7(8): e44080, 2012; Burkhardt et al., Journal of Immunology; 188(12): 6399-6406, 2012; Lee et al., American Journal of Physiology Endocrinology and Metabolism 304: E32-E40, 2013; Maravillas-Montero et al., J Immunol. 2015 Jan. 1; 194(1):29-33) and include chemotaxis, calcium mobilization, transcriptional response, bacteriocidal, and signaling assays. In one embodiment, neutralizing antibodies are characterized using a calcium mobilization assay that reflects the activity of the CXCL17 receptor in the unmodified human monocytic cell line, THP1, as well as in engineered cell lines (Wang et al., The Journal of Biological Chemistry; 281(31) 22021-22028, 2006; Lee et al., American Journal of Physiology Endocrinology and Metabolism 304: E32-E40, 2013; Maravillas-Montero et al., J Immunol. 2015 Jan. 1; 194(1):29-33). Calcium mobilization assays can generally be run in a high-throughput format and are likely to be compatible with crude phage scFv-containing peripreps. Neutralizing antibodies selected as described above are sequenced and unique clones analyzed. Neutralizing antibodies will be screened for poly-reactivity and poly-reactive binders excluded. Neutralizing antibodies will also be epitope binned and selected hits from each bin will be assayed for CXCL17 binding and neutralization activity. Promising scFv clones with the appropriate characteristics will be reformatted into fully human IgGs engineered to be devoid of cytolytic and cytotoxic function (e.g., effector function null).

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

In various embodiments, an antibody that binds CXCL17 is monoclonal. Alternatively, the anti-CXCL17 antibody is a polyclonal antibody. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies. Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody.

Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes. The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

### GPR35 Antagonists

CXCL17 binds to G protein coupled receptor, GPR35 (Maravillas-Montero et al., J Immunol. 2015 Jan. 1; 194(1):29-33). GPR35-CLXL17 mediated functional responses have been reported in the 50 nM range (Maravillas-Montero et al., supra). Agents that antagonize GPR35 would likely prevent chemotaxis of CD11b+Ly6G+Ly6Clo cells to tumors and inhibit tumor growth. GPR35 antagonists have been described (Wang et al., The Journal of Biological Chemistry; 281(31) 22021-22028, 2006; Tanaguchi et al., FEBS Letters; 580(21):5003-5008, 2006; Zhao et al., Molecular Pharmacology; 78(4): 560-568, 2010; Jenkins et al., The Journal of Pharmacology and Experimental Therapeutics; 343(3): 683-695, 2012). Moreover, ML145 and CID2745687 have been shown to have in vitro antagonist activity at both human and mouse GPR35 (Zhao et al., Molecular Pharmacology; 78(4): 560-568, 2010; Jenkins et al., The Journal of Pharmacology and Experimental Therapeutics; 343(3): 683-695, 2012) and are, therefore, expected to be useful for the treatment of cancer that is PTEN and p53 deficient.

### Inhibitory Nucleic Acids

Inhibitory nucleic acid molecules are those oligonucleotides that inhibit the expression or activity of a CXCL17 polypeptide. Such oligonucleotides include single and double stranded nucleic acid molecules (e.g., DNA, RNA, and analogs thereof) that bind a nucleic acid molecule that encodes a CXCL17 polypeptide (e.g., antisense molecules, siRNA, shRNA) as well as nucleic acid molecules that bind directly to the polypeptide to modulate its biological activity (e.g., aptamers). In one embodiment, the invention provides a viral-mediated shRNA approach to knock down Cxcl17 expression in Pten^{pc-/-}; Trp53^{pc-/-} tumors.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer.

The inhibitory nucleic acid molecules of the present invention may be employed as double-stranded RNAs for RNA interference (RNAi)-mediated knock-down of expression. In one embodiment, expression of CXCL17 polypeptide is reduced in a subject having cancer that is PTEN and p53 deficient. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes. shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+ envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo (e.g., a nucleic acid encoding CXCL17). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Therapeutic Methods

The methods and compositions provided herein can be used to treat or prevent progression of a cancer characterized by the loss of a tumor suppressor (e.g., Pten, Zbtb7a/Pokemon, p53, Pml). In particular embodiments, a CXCL17 antagonist is used to treat prostate cancer, breast cancer, colorectal cancer, gastric cancer, pancreatic cancer, or any other cancer of epithelial origin that is deficient in Pten and Zbtb7a/Pokemon or p53. In general, antibodies specific to a CXCL17 polypeptide can be administered therapeutically and/or prophylactically.

Treatment will be suitably administered to subjects, particularly humans, suffering from, having, susceptible to, or at risk of developing such cancer. Determination of those subjects “at risk” can be made by any objective or subjective determination by a diagnostic test or opinion of a subject or health care provider (e.g., genetic test, enzyme or protein marker, family history, and the like). The methods herein also include administering to the subject (including a subject identified as in need of such treatment) an effective amount of an anti-CXCL17 antibody as described herein. Identifying a subject in need of such treatment can be in the judgment of a subject or a health care professional and can be subjective (e.g. opinion) or objective (e.g. measurable by a test or diagnostic method).

In some aspects, the invention features methods of treating or preventing cancer in a subject, the methods comprising administering to the subject an effective amount of a composition comprising an anti-CXCL17 antibody. Optionally, an anti-CXCL17 therapeutic of the invention (e.g., an anti-CXCL17 antibody as described herein) may be administered in combination with one or more of any other standard anti-cancer therapies. For example, an anti-CXCL17 antibody as described herein may be administered in combination with standard chemotherapeutics. Methods for administering combination therapies (e.g., concurrently or otherwise) are known to the skilled artisan and are described for example in Remington's Pharmaceutical Sciences by E. W. Martin.

### Pharmaceutical Compositions

The present invention features compositions useful for treating cancer that is characterized by the loss of a tumor suppressor (e.g., Pten, Zbtb7a/Pokemon, p53, Pml). The methods include administering an effective amount of a CXCL17 antibody or other agent that inhibits CXCL17 expression or activity provided herein to an individual in a physiologically acceptable carrier.

Typically, the carrier or excipient for the composition provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with the composition of the invention.

The administration of a composition comprising an anti-CXCL17 antibody or other agent herein (e.g., agent that inhibits CXCL17 expression or activity) for the treatment or prevention of cancer may be by any suitable means that results in a concentration of the therapeutic that, combined with other components, is effective in ameliorating, reducing, or stabilizing the disease symptoms in a subject. The composition may be administered systemically, for example, formulated in a pharmaceutically-acceptable buffer such as physiological saline. Preferable routes of administration include, for example, subcutaneous, intravenous, intraperitoneally, intramuscular, intrathecal, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of cancer, although in certain instances lower amounts will be needed because of the increased specificity of the agent. A composition is administered at a dosage that ameliorates or decreases effects of the cancer as determined by a method known to one skilled in the art.

The therapeutic or prophylactic composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intrathecally, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent to a particular cell type. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac dysfunction or disease, the composition may include suitable parenterally acceptable carriers and/or excipients. The active therapeutic agent(s) (e.g., an anti-CXCL17 agent described herein) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic (i.e., an anti-CXCL17 antibody herein) is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Murine Platform for Screening CXCL17 Therapies

The invention further provides methods for characterizing anti-CXCL17 therapies in immunocompromised mice that are implanted with human tumor cell lines or primary human tumors (PDX models) (FIG. 1). In particular embodiments, an implanted tumor constitutively over-expresses CXCL17, is engineered to over-express CXCL17, or is engineered to have reduced (e.g. via shRNA knockdown) CXCL17 (Matsui et al., PLoS ONE; 7(8): e44080 2012). Such immunocompromised mice generally lack adaptive immune system components, but have relatively intact innate immune systems. Therefore, upon tumor formation, infiltration of mouse MDSCs is assessed along with their phenotypic characteristics (immunosuppressive markers, cell surface markers, immunosuppressive potency). A similar approach is taken with mouse tumor lines in syngenic hosts. In either xenograft or syngenic models, tumor cell lines overexpressing human or mouse CXCL17 are assessed. Such mice are used to assess the biological response to neutralizing antibodies or other anti-CXCL17 therapies. For example, the effects of anti-CXCL17 antibody administration is evaluated by assaying tumor vascularization (Matsui et al., supra), the profile of tumor infiltrating MDSCs and other immune cells, correlations of CXCL17 expression levels with changes in Treg numbers or Th1 versus Th2 cytokine profiles, tumor growth, and/or murine survival.

Pathological expression of CXCL17 by human tumors recruits immunosuppressive myeloid cells to the tumor microenvironment. Accordingly, the effect of anti-CXCL17 antibodies on MDSCs is assessed. A chemotaxis assay (e.g. transwell assay) is used to assay the effects of anti-CXCL17 antibodies on MDSC migration. Primary MDSCs can be obtained from the Pten−/−; Trp53−/− mouse model or from human patients.

In another embodiment, mice are implanted with organoids that either endogenously express CXCL17 or are engineered to do so. Methods for generating organoids are known in the art and described, for example, by Boj et al., Cell; 160: 324-338, 2015; Gao et al., Cell; 159: 176-187, 2014; Linde et al., PLoS ONE; 7(7): e40058, 2012. In another embodiment, organoids are maintained in co-culture with autologous PBMC using tumor tissue and PBMCs from the same human patient.

The GEMM platform can be used with virtually any murine model known in the art. In one embodiment, therapies described herein are evaluated in a Cxcl17 conditional knockout mouse that is part of the KOMP collection (https://www.komp.org/geneinfo.php?geneid=29373 (Burkhardt et al., 2012). This conditional allele will be used to generate a prostate-specific Pten−/−; Trp53−/− lacking all 3 genes in the prostatic epithelium. In another embodiment, primary tumors from the Pten−/−; Trp53−/− model will be implanted into CXCL17 receptor (Gpr35; Maravillas-Montero et al., J Immunol. 2015 Jan. 1; 194(1):29-33) knockout mice, which are also available as part of the KOMP collection.

### Patient Stratification

Despite advances in pharmacology, many cancer patients fail to benefit from standard anti-cancer therapies or experience an adverse reaction to the medication they receive. There are many subsets of patients who suffer from apparently similar clinical diseases, but whose molecular underpinnings are different. Failure to quickly identify such patients delays appropriate efficacious therapy and allows their cancers to advance unchecked. The present invention provides insights into the disease mechanisms and drug actions underlying tumor suppressor (e.g., Pten, Zbtb7a/Pokemon, p53, Pml) deficient cancers. In this context, the present invention provides for the identification of subjects having a cancer characterized by the loss of a tumor suppressor (e.g., Pten, Zbtb7a/Pokemon, p53, Pml). In one embodiment, Pten and Zbtb7a/Pokemon and/or p53 can be used as biomarkers to identify patients that are responsive to anti-CXCL17 therapy. Biomarkers Pten and Zbtb7a/Pokemon and/or p53 can be used to stratify different patient groups in terms of clinical response, so as to develop personalized, preventive or therapeutic strategies. Accordingly, the invention provides a method for characterizing a tumor suppressor (e.g., Pten, Zbtb7a/Pokemon, p53, Pml) in a biological sample obtained from the subject where the identification of reduced or undetectable levels of tumor suppressor expression indicates that the subject would benefit from to anti-CXCL17 therapy.

The invention provides for the integration of a particular treatment (administration of an effective amount of anti-CXCL17 antibodies) into the diagnostic and treatment process. The combination of the diagnostic and therapeutic steps is not routine and conventional, but ensures that patients who have a particular type of cancer (e.g., deficient in Pten and Zbtb7a/Pokemon and/or p53) will be accurately diagnosed (and properly treated with anti-CXCL17 antibodies), as opposed to being misdiagnosed and administered a therapy that is ineffective.

The present invention provides methods of treating Pten and Zbtb7a/Pokemon and/or p53 deficient cancer or symptoms thereof which comprise administering a therapeutically effective amount of a pharmaceutical composition comprising an anti-CXCL17 antibody or agent that otherwise inhibits the expression or activity of CXCL17 herein to a subject (e.g., a mammal such as a human). The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

In one embodiment, the invention provides a method of monitoring treatment progress. The method includes the step of determining a level of diagnostic marker (Marker) (e.g., any target delineated herein modulated by a compound herein, a protein or indicator thereof, including a tumor suppressor or diagnostic measurement (e.g., screen, assay) in a subject suffering from or susceptible to a disorder or symptoms thereof associated with a tumor suppressor deficient cancer, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Kits

The invention provides kits for the treatment or prevention of cancer. In some embodiments, the kit includes a therapeutic or prophylactic composition containing an effective amount of an anti-CXCL17 agent (e.g., an anti-CXCL17 antibody) in unit dosage form. In other embodiments, the kit includes a therapeutic composition containing an effective amount of an anti-CXCL17 agent in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof, precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: The Genetic Make-Up of Prostate Cancer Dictates the Composition of Immune Infiltrates in the Primary Tumor

To address whether the genetic make-up of cancer impacts the components of the TME, the “Co-Clinical platform” was utilized as described by Chen, Z. et al. (Nature. 2005. 436, 725-30), in which genetically engineered mouse models (GEMMs) driven by distinct genetic alterations are systematically analyzed, at a steady state or upon therapeutic perturbations. As Pten is one of the most frequently lost and relevant tumor suppressors in prostate cancer, genetic complexity representative of human prostate cancer was added to the non-lethal Pten-loss driven mouse model (Pten^{Lx/Lx}; Probasin-Cre, prostate specific loss of PTEN; referred to herein as Ptenpc−/−). To this end, the data generated by the experiments of this example characterized the composition of the immune cells of Pten^{Lx/Lx}; Pml^{Lx/Lx} Probasin-Cre (referred to as Pten^{pc-/-}; Pml^{pc-/-}); Pten^{pc-/-}; Zbtb7a^{Lx/Lx} Probasin-Cre (referred to as Pten^{pc-/-} Zbtb7a^{pc-/-}) and Pten^{Lx/Lx}; Trp53^{Lx/Lx} Probasin-Cre (referred to as Pten^{pc-/-} Trp53^{pc-/-} mice, all displaying very aggressive phenotypes.

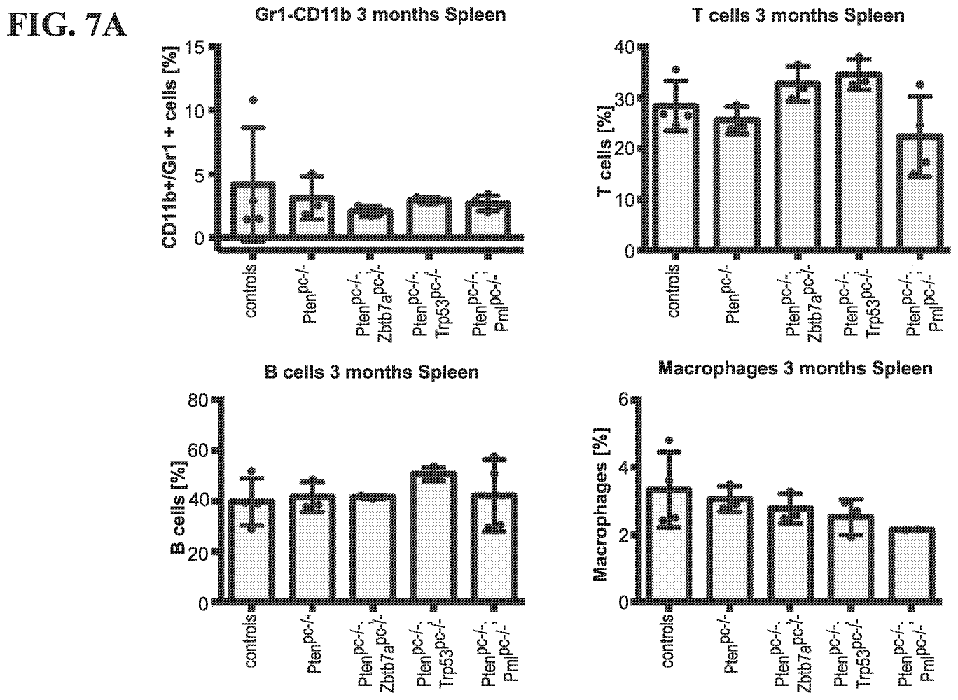

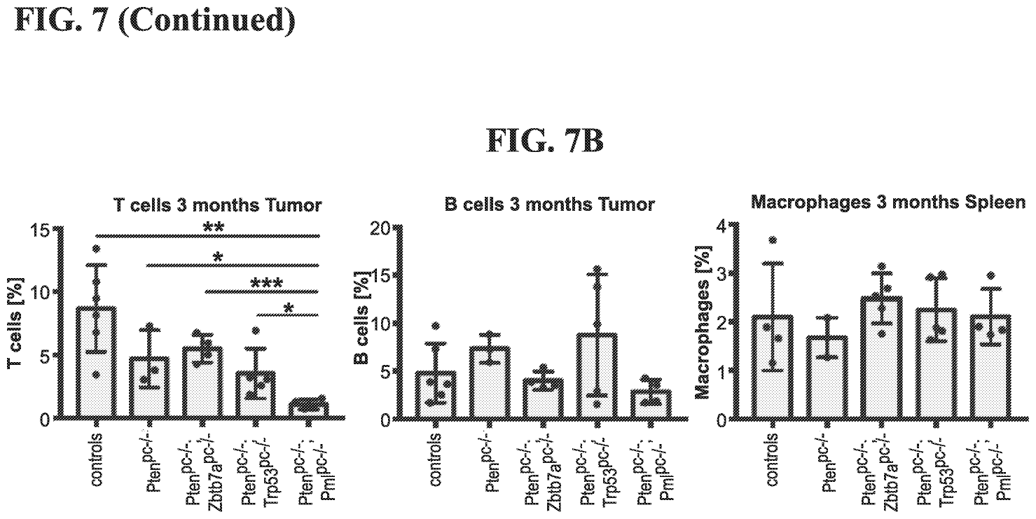

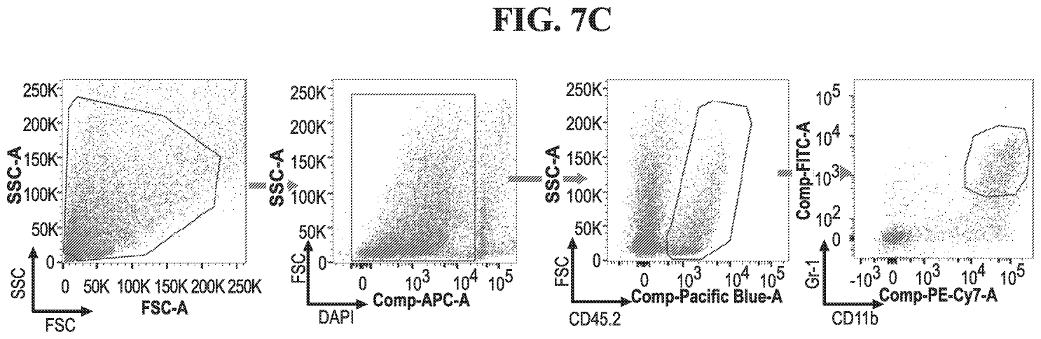

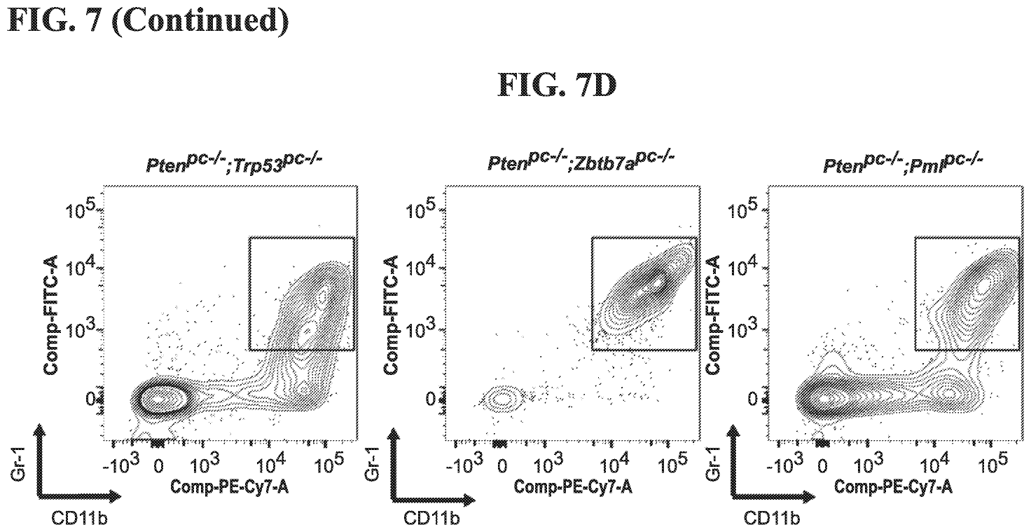

The experiments of this example first analyzed T cells (CD3+), B cells (CD19+/B220+), macrophages (CD11b+/F480+) and Gr-1+/CD11b+ myeloid cells (immature myeloid cells, monocytes, neutrophils) in whole prostate tumor tissue single cell suspensions at 3 months of age. At this age, all of the analyzed GEMMs developed high-grade prostatic intraepithelial neoplasia with partially locally invasive prostatic adenocarcinoma only observed in Pten^{pc-/-} Zbtb7a^{pc-/-}, Pten^{pc-/-}; Trp53^{pc-/-} and Pten^{pc-/-}; Pml^{pc-/-} mice (FIG. 1A and FIG. 1B, black arrows). The presence of the aforementioned immune cell populations were further analyzed in the spleen, a classical hematopoietic organ, to assess whether tumor-bearing mice display altered immune cell populations in the periphery. While changes in the spleen between control and tumor bearing mice were not detected, or between different models (FIG. 7A), the profiling of primary tumor tissue showed profound differences in the immune cell infiltrates in the various GEMMs (FIG. 1C and FIG. 7B). Most strikingly, Gr-1+/CD11b+ cells (FIG. 1C, FIG. 1D, FIG. 7C, and FIG. 7D) varied significantly between the control and tumor bearing mice as well as between the different models. Consistent with a previous report (Di Mitri, D. et al. Nature 515, 134-137 (2014)), the infiltration of Gr-1+/CD11b+ cells were increased in Pten^{pc-/-} prostate tumors as compared to control prostates, indicating a clear interaction between primary prostate cancer and Gr-1+/CD11b+ cells. Moreover, compound loss of Zbtb7a or p53 dramatically increased the accumulation of Gr-1+/CD11b+ cells, especially in Pten^{pc-/-}; Zbtb7a^{pc-/-}-mice when compared to Pten^{pc-/-} mice. By contrast, the infiltration of Gr-1+/CD11b+ cells as well as T cells was decreased in Pten^{pc-/-}; Pml^{pc-/-} compared to the other models (FIG. 1C, FIG. 1D, and FIG. 7B), even though the Pten^{pc-/-}; Pml^{pc-/-} mice also developed very aggressive and lethal prostate cancers (FIG. 1A, and FIG. 1B).

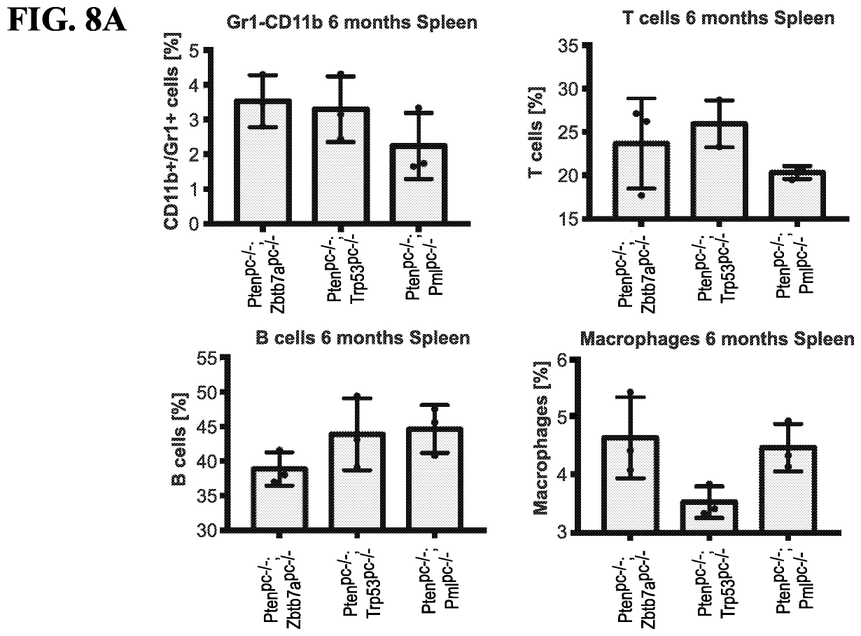

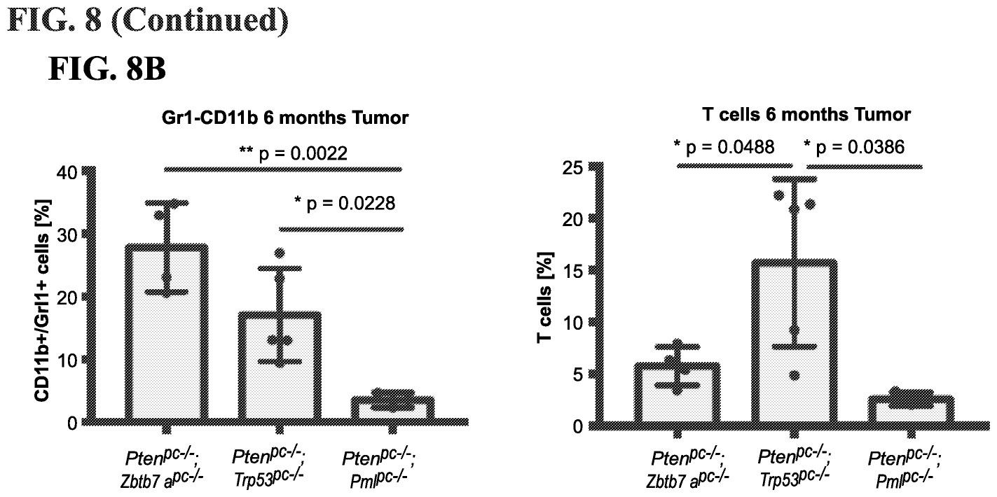

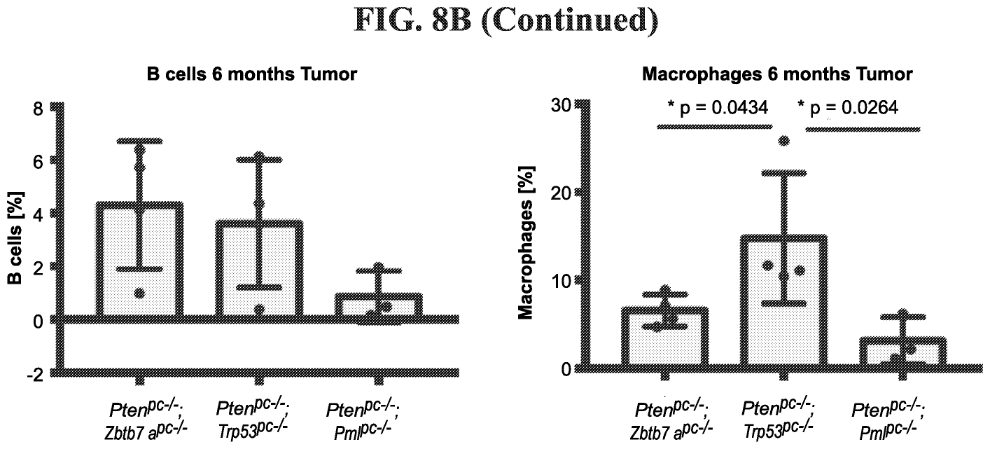

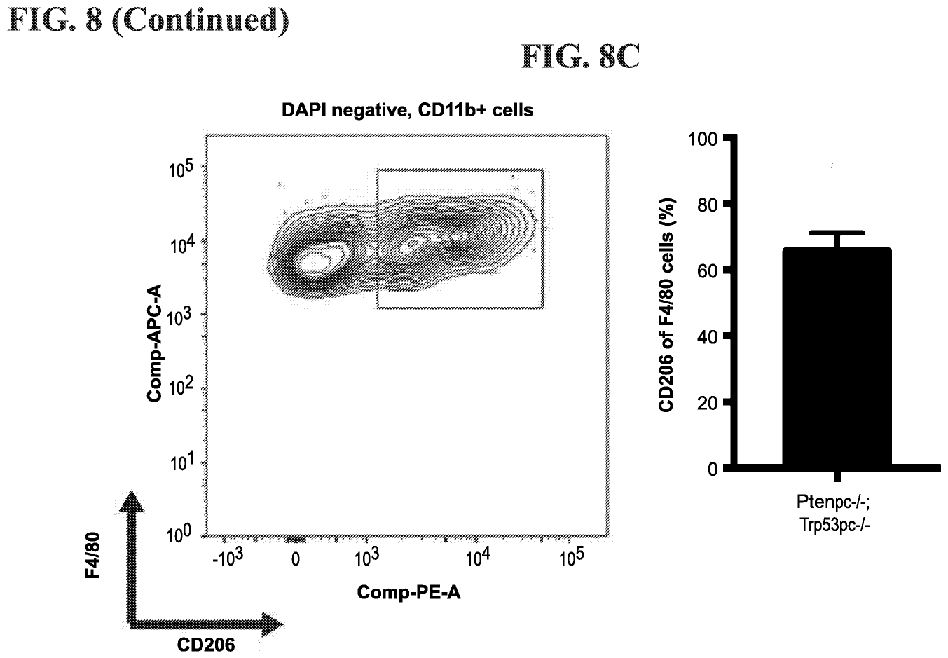

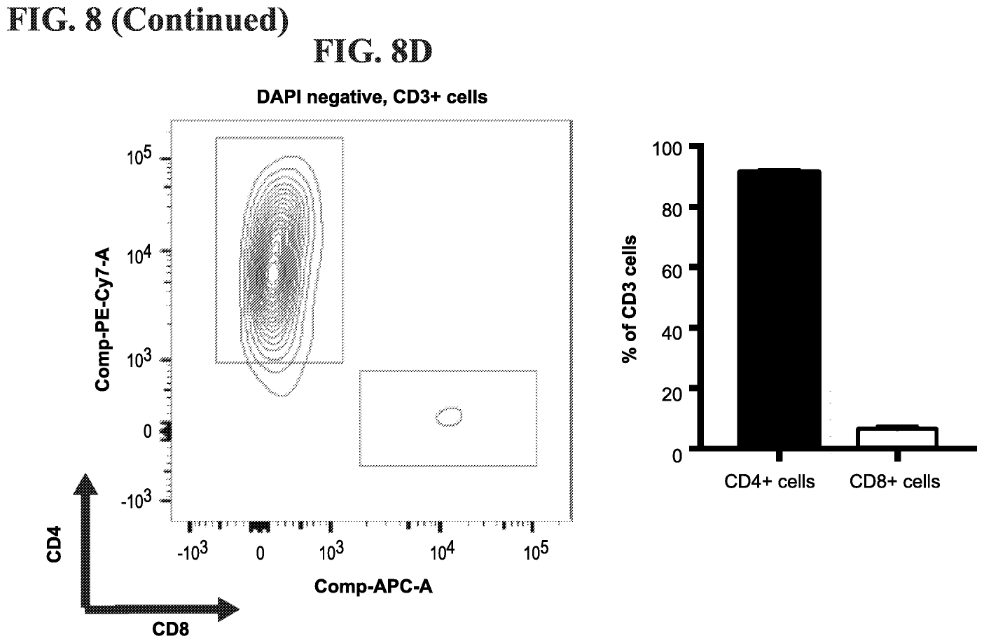

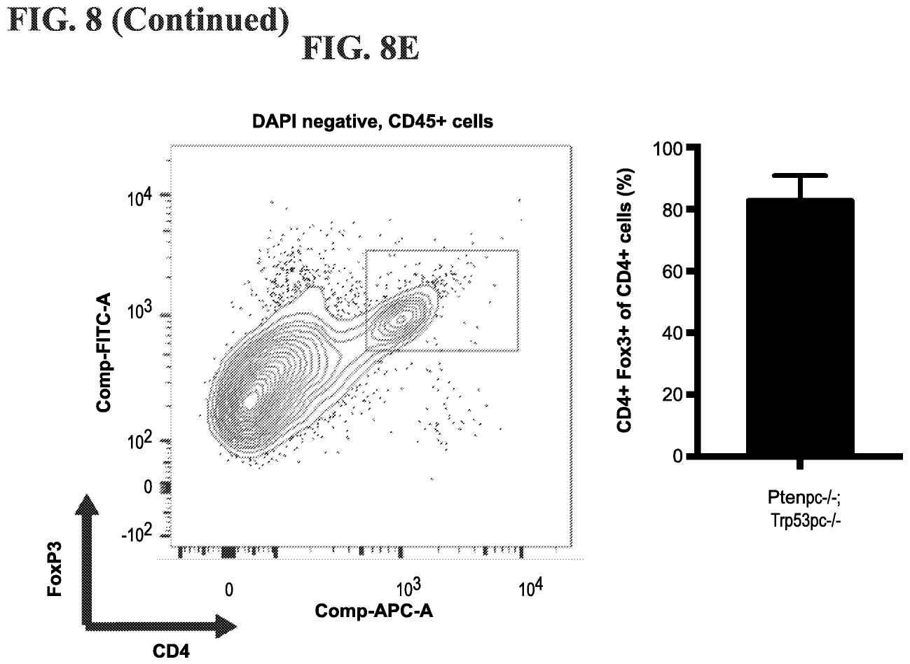

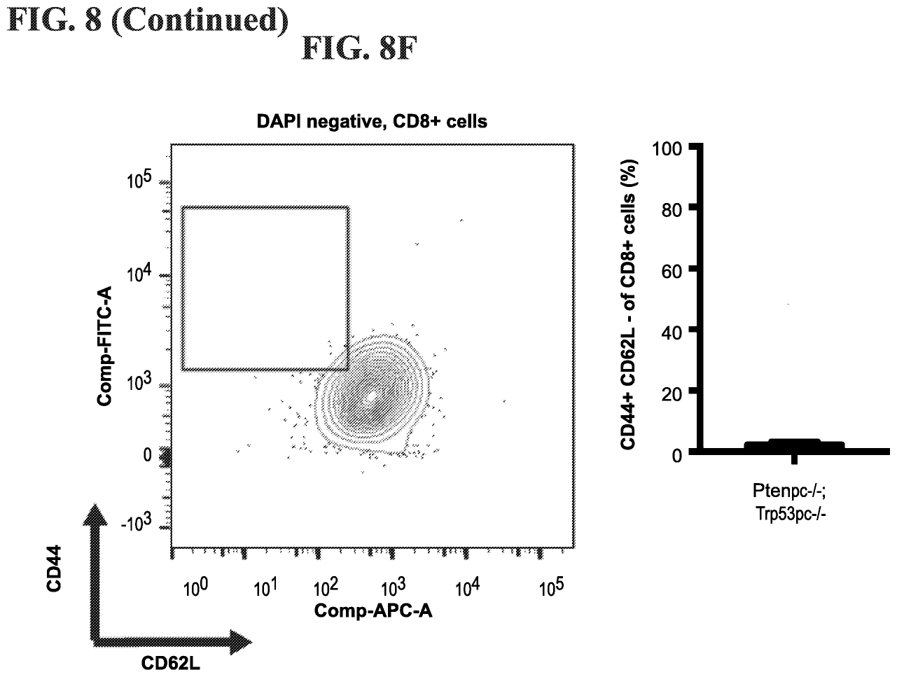

Next, in order to understand how the immune landscape evolves during disease progression the experiments of this example analyzed prostate cancer models at 6 months of age. At this stage, the Pten^{pc-/-}; Trp53^{pc-/-} mice displayed a much larger tumor compared to the Pten^{pc-/-}; Zbtb7a^{pc-/-} and Pten^{pc-/-}; Pml^{pc-/-} models (FIG. 1E). Remarkably, major changes in the spleen of these tumor-bearing mice were not detected (FIG. 8A), the immune landscapes of the three models diverged even further in accordance with the profiles observed at 3 months of age (FIG. 1F, and FIG. 8B). The Pten^{pc-/-}; Pml^{pc-/-} tumors still appeared “immune-depleted”, whereas the Pten^{pc-/-}; Zbtb7a^{pc-/-} immune landscape was dominated with Gr-1+/CD11b+ cells. The Gr-1+/CD11b+ cell population was also increased in Pten^{pc-/-}; Trp53^{pc-/-} mice along with a marked recruitment of T cells and macrophages. Interestingly, further analysis revealed that the majority of macrophages had an M2-like phenotype (CD11b+/F4/80+/CD206+) (FIG. 8C) and that the increase of CD3+ cells was reflecting the recruitment of CD4+/FoxP3+ T regulatory cells (Treg) able to suppress cytotoxic T cells, defining a potentially favorable microenvironment for cancer immune-evasion (FIG. 8D, FIG. 8E, and FIG. 8F). These data indicate that the genetic background of prostate cancer determines the composition of the immune infiltration in the primary tumor, and specifically impacts the population of Gr-1+/CD11b+ cells infiltrating the tumor site, irrespective of the histological characteristics of the primary tumoral lesion.

### Example 2: Characterization of Gr-1+/CD11b+ Cells in Pten^{pc-/-}; Zbtb7a^{pc-/-} and Pten^{pc-/-}; Trp53^{pc-/-} Prostate Tumors

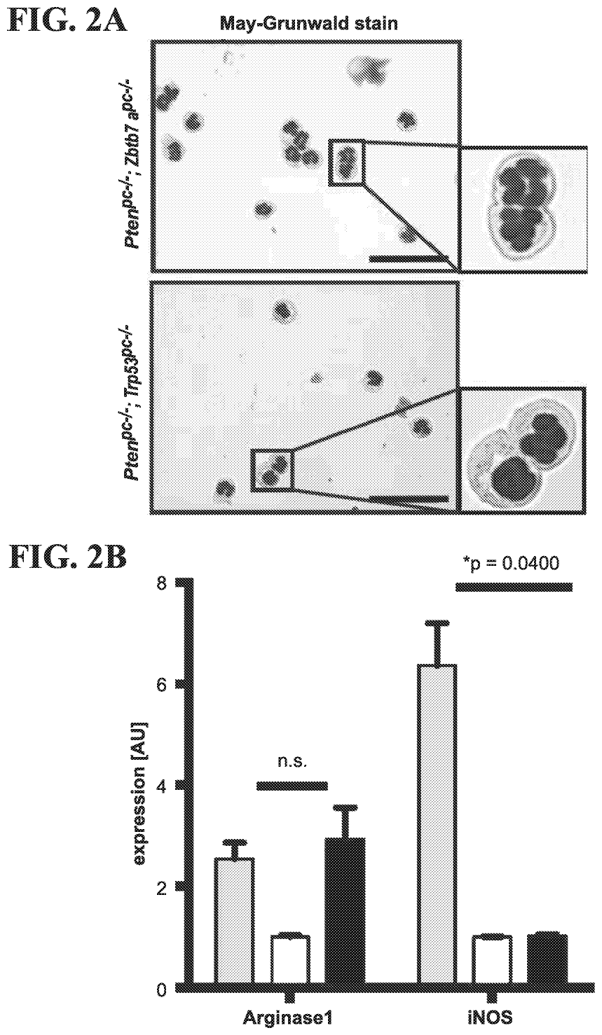

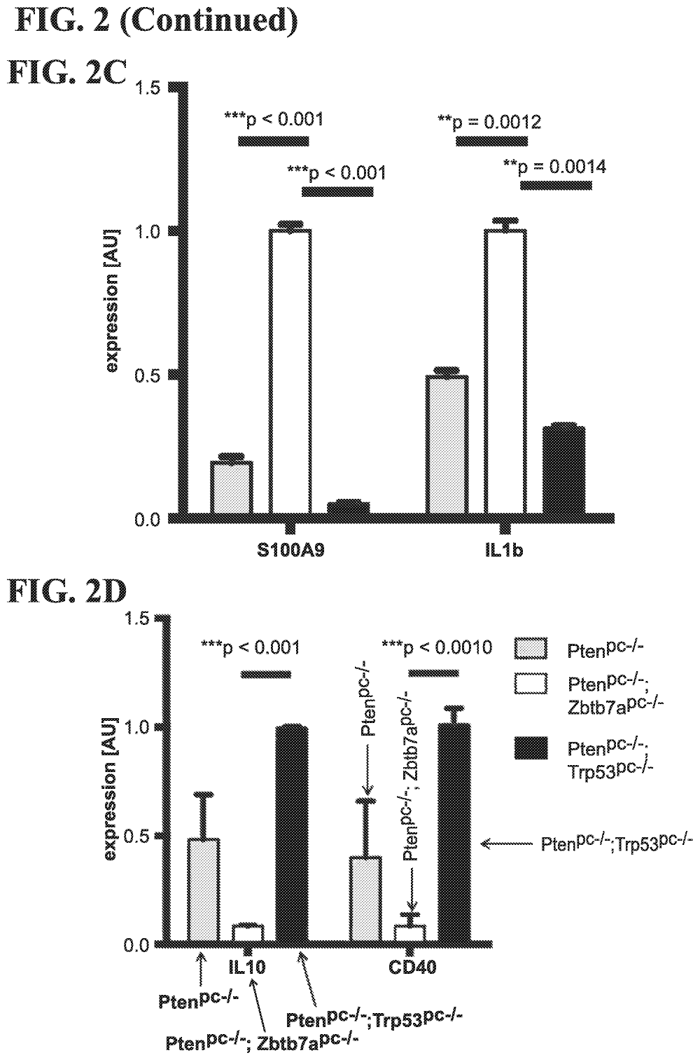

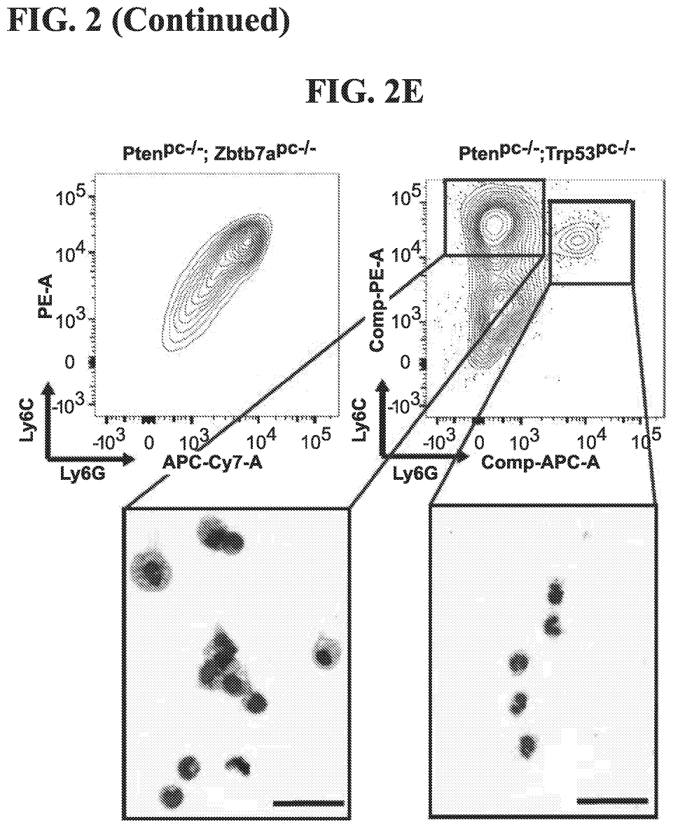

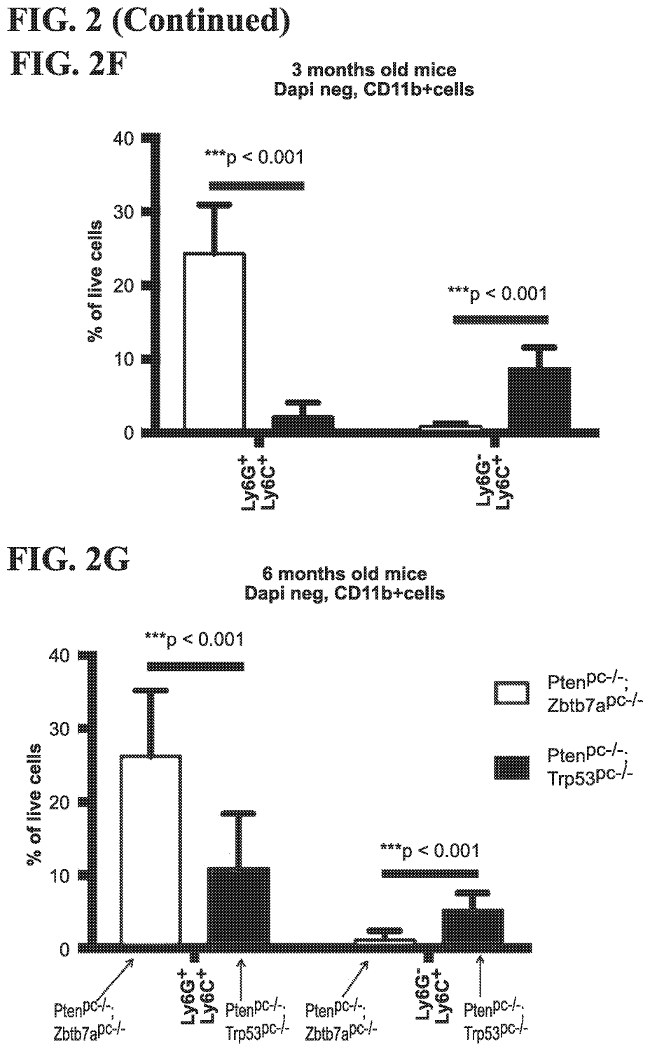

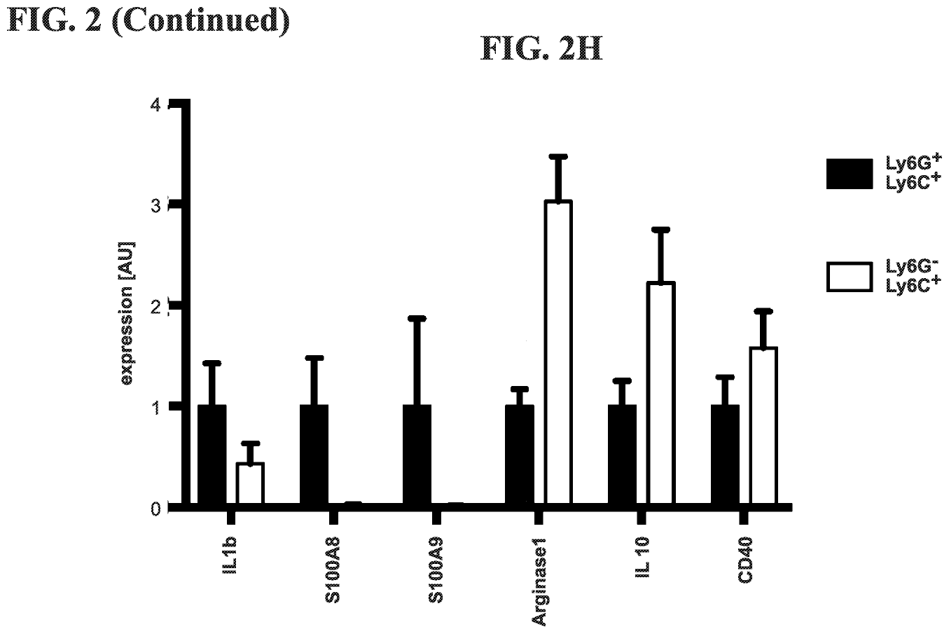

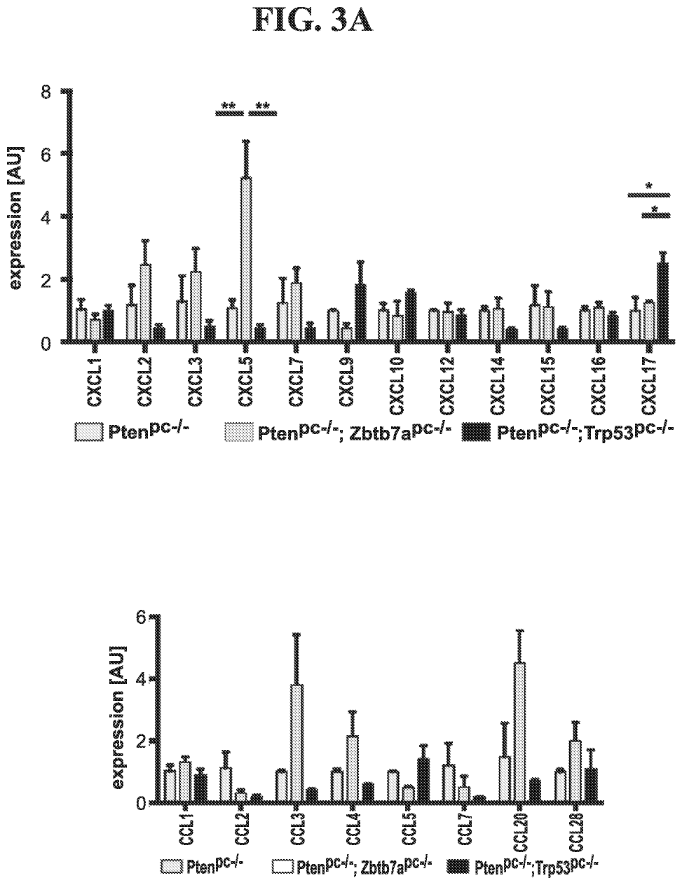

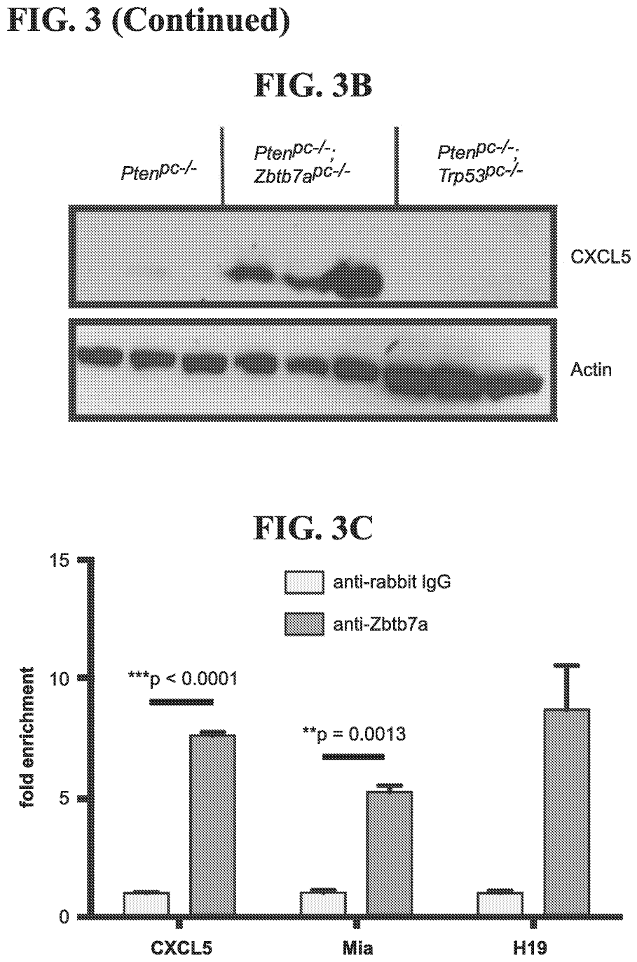

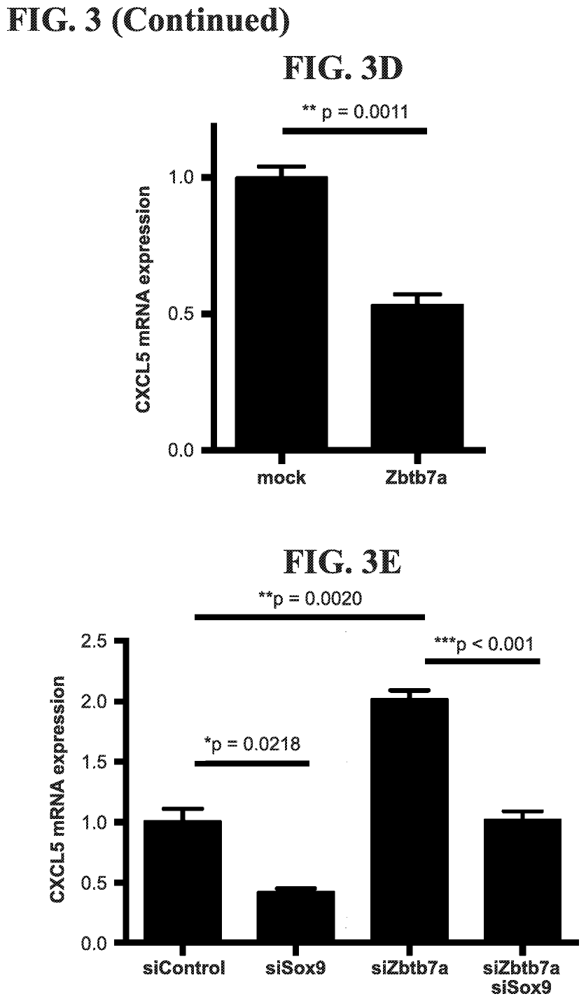

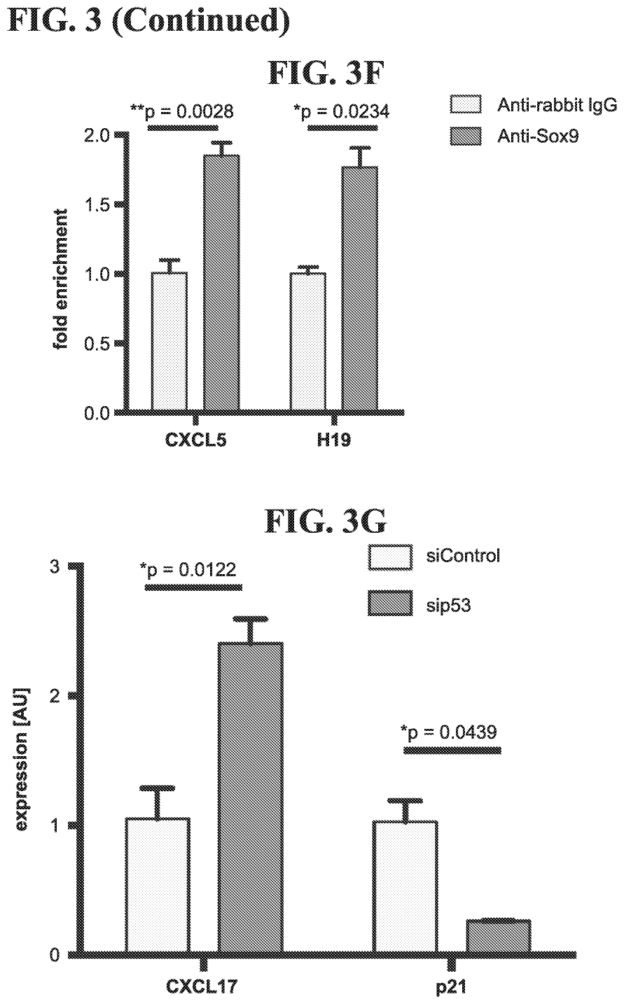

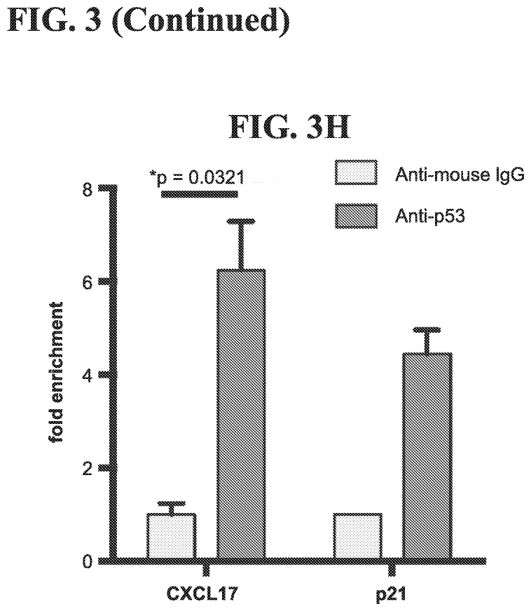

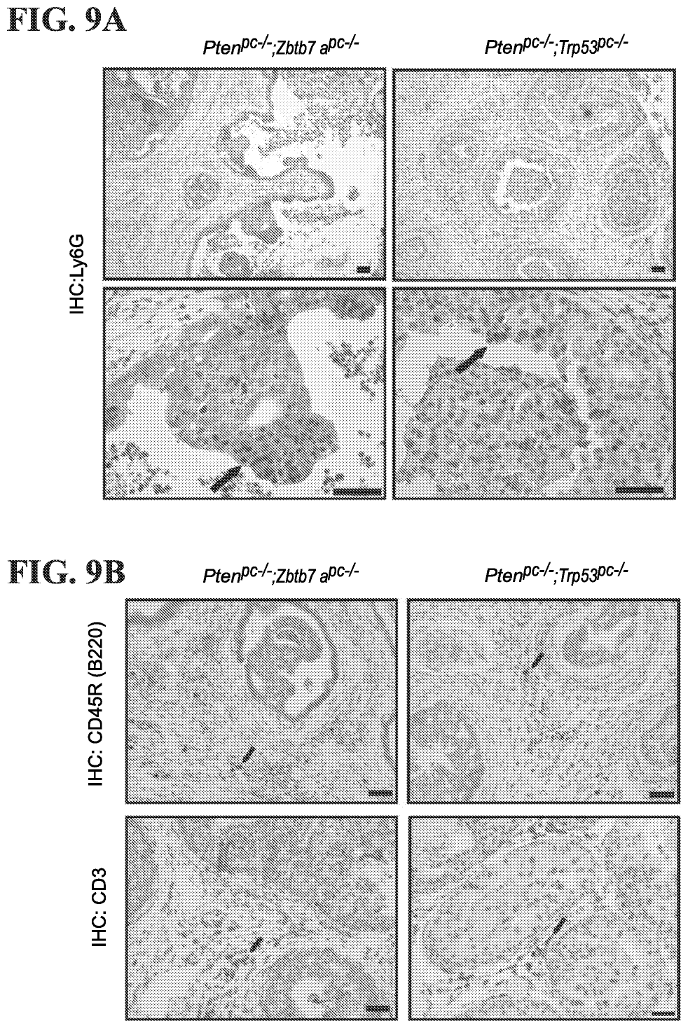

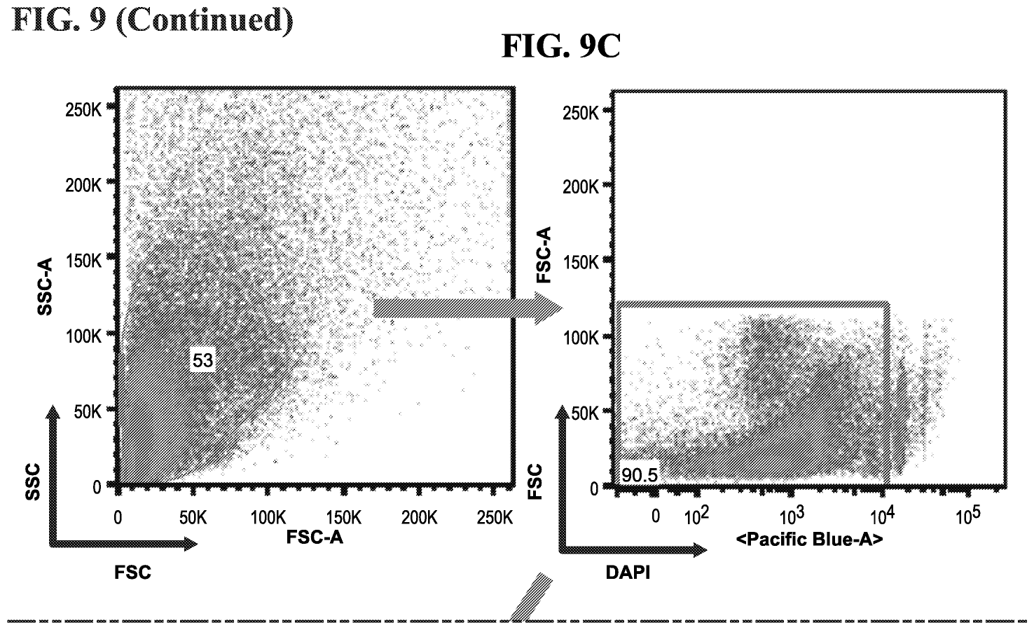

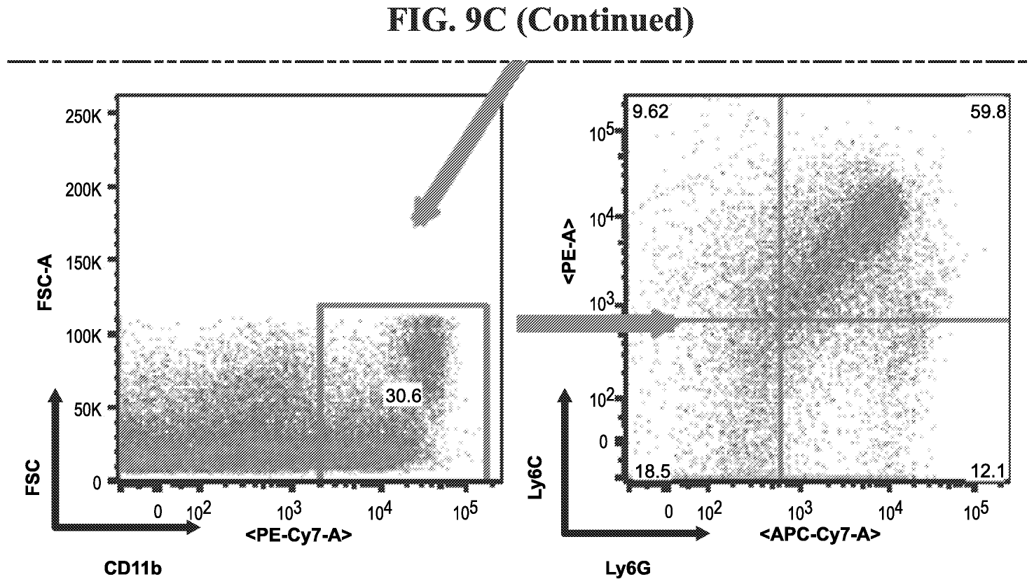

The population of Gr-1+/CD11b+ cells is heterogeneous and comprises mature neutrophils, monocytes and immature myeloid cells (iMC). The latter, when able to suppress cytotoxic T cells, are functionally classified as myeloid derived suppressor cells (MDSCs). MDSCs can be further divided into polymorphonuclear MDSCs (PMN-MDSCs) and monocytic MDSCs (Mo-MDSCs) based on morphological analysis and on the expression of the markers Ly6C and Ly6G. The experiments of this example demonstrated that the morphology of the tumor infiltrated Gr-1+/CD11b+ cells in the two models showed the highest levels of infiltration of myeloid cells at 3 months of age (FIG. 2A). This analysis verified the partly hyper-segmented granulocytic phenotype of the Gr-1+/CD11b+ cells in Pten^{pc-/-}; Zbtb7a^{pc-/-} prostate tumors, distinctive of PMN-MDSCs and neutrophils. In contrast, the Pten^{pc-/-}; Trp53^{pc-/-} infiltrated Gr-1+/CD11b+ cell population appeared heterogeneous and included both polymorphonuclear and mononuclear cells. The localization of these cells was determined through immunohistochemistry (IHC) of the Ly6G epitope (Supp. FIG. 3a). This analysis revealed that this cell population resides mainly in the intra-epithelium of Pten^{pc-/-}; Zbtb7a^{pc-/-} and Pten^{pc-/-}; Trp53^{pc-/-} tumors. Strikingly, compared to IHC of other immune cell infiltrates (FIG. 9B) that were primarily located in the stroma, only Ly6G+ cells were detected in close proximity to tumor cells.

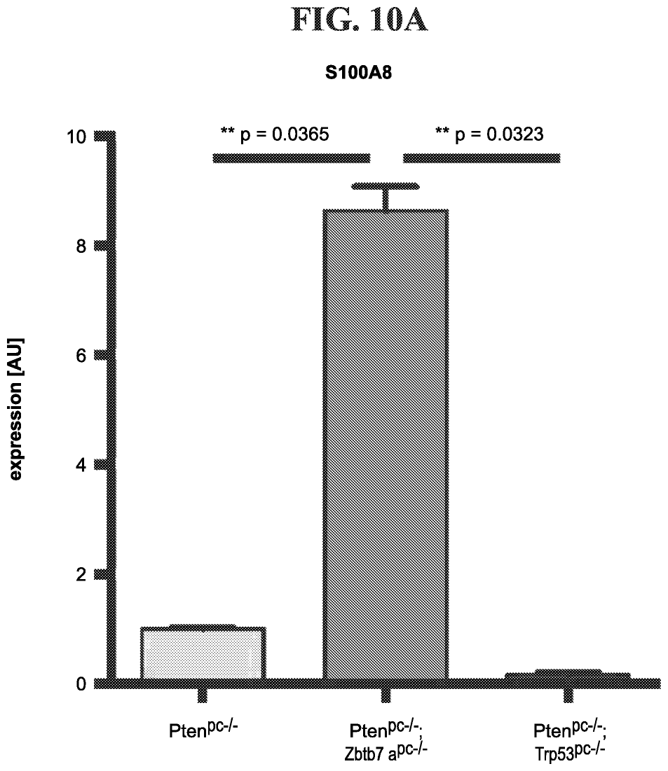

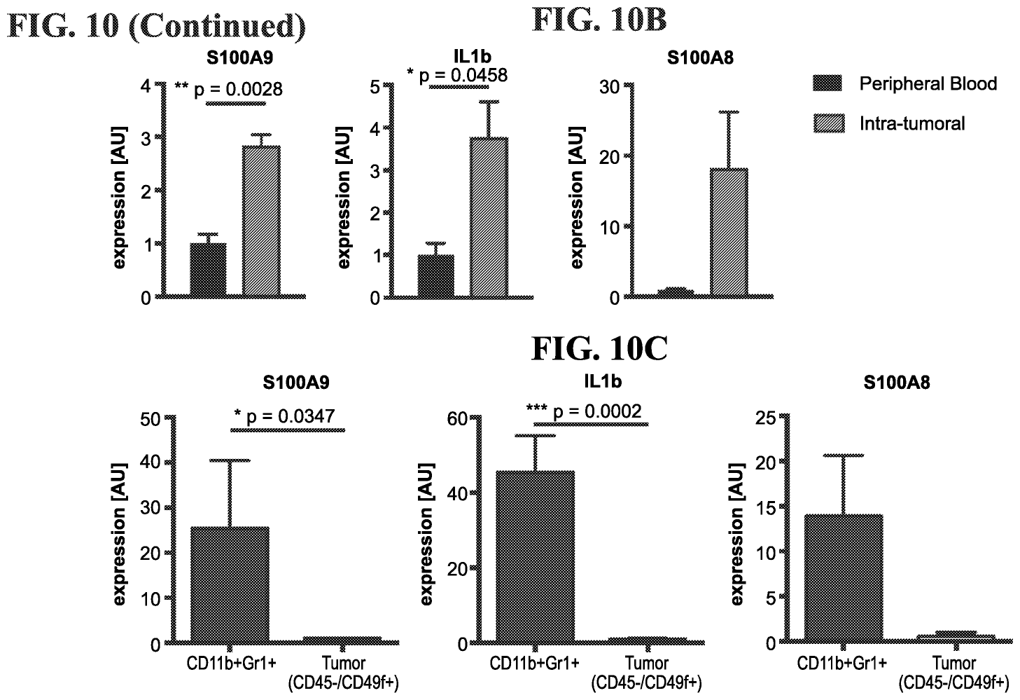

Next, the experiments of this example examined the expression level of a panel of genes implicated in the pro-tumoral function of myeloid cells. The Gr-1+/CD11b+ cells in Pten^{pc-/-} mice were recently shown to support prostate tumors by opposing senescence response and also through classical immune suppression via Arginase 1 (ARG1) and inducible nitric oxidase (iNOS) expression (Di Mitri, D. et al. Nature 515, 134-137 (2014); Garcia, A. J. et al. Mol. Cell. Biol. 34, 2017-2028 (2014)). Interestingly, Gr-1+/CD11b+ cells sorted from Pten^{pc-/-}; Zbtb7a^{pc-/-} tumors showed low expression of Arg1 as well as iNOS, whereas Gr-1+/CD11b+ cells from Pten^{pc-/-}; Trp53^{pc-/-} tumors showed high expression of Arg1 and low expression of iNOS when compared to Gr-1+/CD11b+ cells sorted from Pten^{pc-/-} tumors (FIG. 2B). Notably, Gr-1+/CD11b+ cells sorted from Pten^{pc-/-}; Zbtb7a^{pc-/-} tumors showed significantly higher expression of the tumor promoting genes S100A9, S100A8 and IL1b when compared to Gr-1+/CD11b+ cells from Pten^{pc-/-} and Pten^{pc-/-}; Trp53^{pc-/-} tumors (FIG. 2C and FIG. 10A). Pten^{pc-/-}; Zbtb7a^{pc-/-} intra-tumoral Gr-1+/CD11b+ cells displayed a specific upregulation of these genes when compared to Gr-1+/CD11b+ cells from the peripheral blood (FIG. 10B) or to CD49f+ tumor cells (mouse prostate basal and luminal cells) (FIG. 10C).

Next, the experiments of this example tested the expression levels of IL10 and CD40, which are both associated with Treg cells activation. They were both upregulated in Gr-1+/CD11b+ cells sorted from Pten^{pc-/-}; Trp53^{pc-/-} tumors, when compared to those sorted from Pten^{pc-/-}; Zbtb7a^{pc-/-} and Pten^{pc-/-} tumors (FIG. 2D), suggesting genotype-specific modes of tumor promotion mediated by myeloid cells. To further characterize the phenotype of these cells, the expression of the Ly6G and Ly6C epitopes was studied (FIG. 2E and FIG. 9C). Strikingly, flow cytometry analysis of primary tumors at 3 months of age revealed that CD11b+ cells in Pten^{pc-/-}; Zbtb7a^{pc-/-} and Pten^{pc-/-}; Trp53^{pc-/-} tumors were significantly different (FIG. 2E, FIG. 2F). While Pten^{pc-/-}; Zbtb7a^{pc-/-} tumors contained primarily CD11b+/Ly6G+/Ly6C cells with immune phenotypic features of PMN-MDCSs/neutrophils, Pten^{pc-/-}; Trp53^{pc-/-} tumors mainly recruit CD11b+/Ly6G−/Ly6C^{hi} cells with immune phenotypic features of Mo-MDSCs/monocytes (Brandau, S. et al. Nature Communications 7, 1-10 (2016)). The myeloid infiltrate of primary tumors of 6 months of age in the Pten^{pc-/-}; Zbtb7a^{pc-/-} mice was still dominated by polymorphonuclear cells (FIG. 2G). By comparison, Pten^{pc-/-}; Trp53^{pc-/-}CD11b+ cells showed an increase in CD11b+/Ly6G+/Ly6C^{int} cells accompanied by a slight decrease in the monocytic population, potentially secondary to differentiation of these cells into macrophages, which are indeed dramatically increased at this time point (FIG. 1G and FIG. 2F). In order to gain additional insights into the role of the monocytic and PMN populations detected in the Pten^{pc-/-}; Trp53^{pc-/-} tumors at 3 months of age, we repeated the aforementioned gene expression analysis in CD11b+/Ly6G−/Ly6C^{hi} and CD11b+/Ly6G+/Ly6C^{int} sorted cells (FIG. 2H). The Ly6G+/Ly6C^{int} cells showed higher expression of S110A8/A9 and IL1b, similar to the Gr-1+/CD11b+ cells collected from Pten^{pc-/-}; Zbtb7a^{pc-/-} tumors, while the Ly6G−/Ly6C^{hi} population emerged as the primary contributor to the elevated levels of the immune suppressive genes Arg1, I110 and CD40.

### Example 3: Genotype Specific Chemokine Expression Pattern are Directly Influenced by Gene Loss in Pten^{pc-/-}; Zbtb7a^{pc-/-} Compared to Pten^{pc-/-}; Trp53^{pc-/-} Tumors

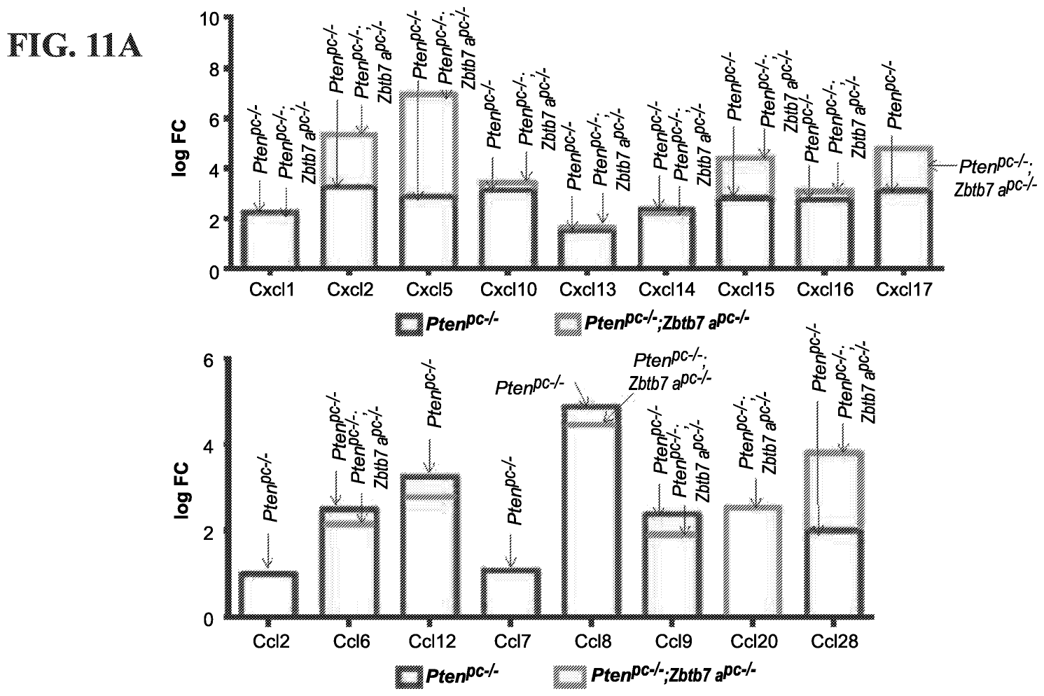

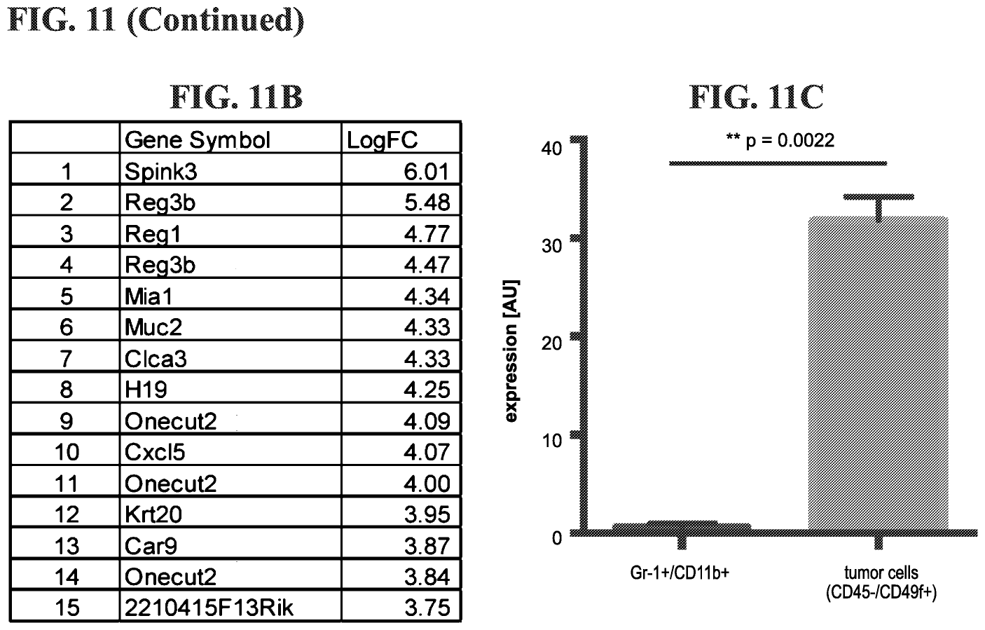

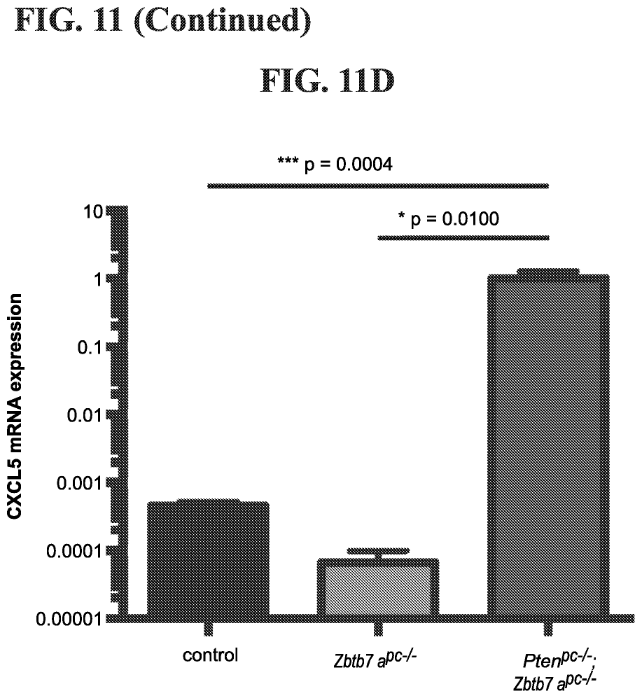

To examine the mechanism of recruitment of Gr1+/CD11b+ cells in an unbiased way, the experiments of this example initially analyzed the available microarray data set for expression of cytokines in Pten^{pc-/-}; Zbtb7a^{pc-/-} compared to Pten^{pc-/-} tumors at 3 months of age. Various cytokines were differentially regulated between the two models and loss of Zbtb7a in a Pten deficient setting leads to the upregulation of a very specific inflammatory program (FIG. 11A, FIG. 11B, FIG. 11C, and FIG. 11D). Based on these data, one aim was to do an mRNA expression analysis of selected chemokines from the CXC family (FIG. 3A left panel) and the CC family (FIG. 3A, right panel), and to compare their expression in Pten^{pc-/-}, Pten^{pc-/-}; Zbtb7a^{pc-/-} and Pten^{pc-/-}; Trp53^{pc-/-} derived prostate tumors. The chemokine analysis experiments revealed a different pattern of inflammatory mediators in Pten^{pc-/-}; Trp53^{pc-/-} tumors compared to Pten^{pc-/-}; Zbtb7a^{pc-/-} tumors (FIG. 3A, FIG. 3B, FIG. 3C, FIG. 3D, FIG. 3E, and FIG. 3F). Specifically, it was found that CXCL17, which was reported as an attractant for monocytic cells (Pisabarro, M. T. et al. The Journal of Immunology 176, 2069-2073 (2006)), was clearly upregulated in Pten; Trp53^{pc-/-} tumors as compared to the other models. Thus, it was investigated whether p53 could transcriptionally suppress CXCL17. To this end, knockdown of p53 expression by siRNA treatment in RWPE1 cells was performed. Intriguingly, knockdown of p53 induced expression of CXCL17 (FIG. 3G). Furthermore, the ability of endogenous p53 to bind the promoter of CXCL17 was confirmed by ChIP analysis in RWPE1 cells (FIG. 3H). In summary, these data suggest that p53 differentially regulates expression of CXCL17 in prostate epithelial cells, and the loss of p53 leads to the specific upregulation of CXCL17 in Pten^{pc-/-}; Trp53^{pc-/-} tumors.

### Example 4: Differential Mechanisms of Gr-1+/CD11b+ Cell Recruitment in Pten^{pc-/-}, Zbtb7a^{pc-/-} Compared to Pten^{pc-/-}; Trp53^{pc-/-} Tumors

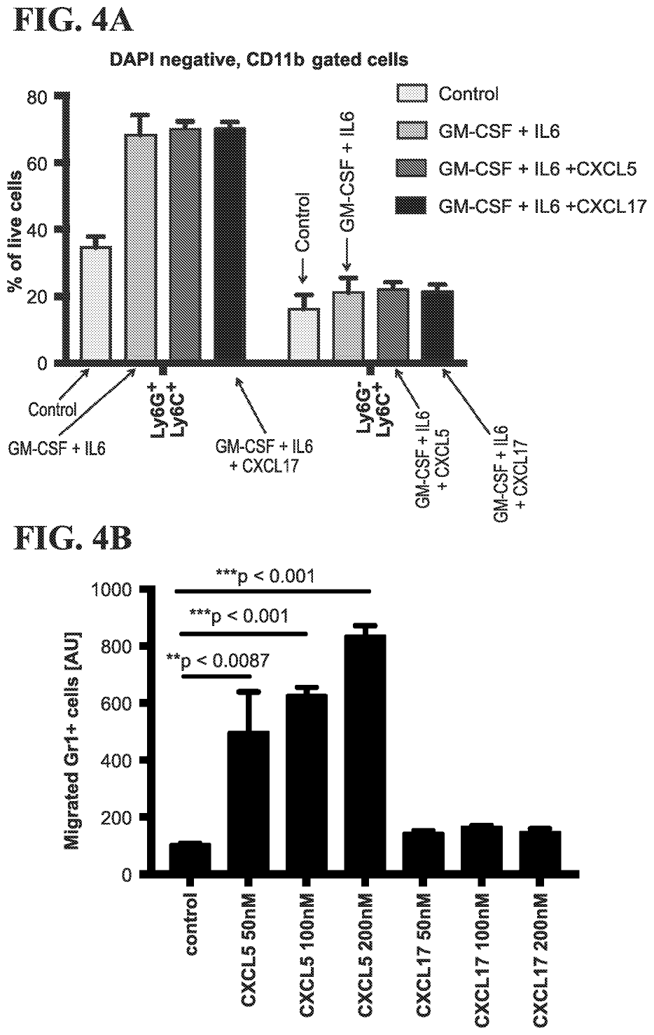

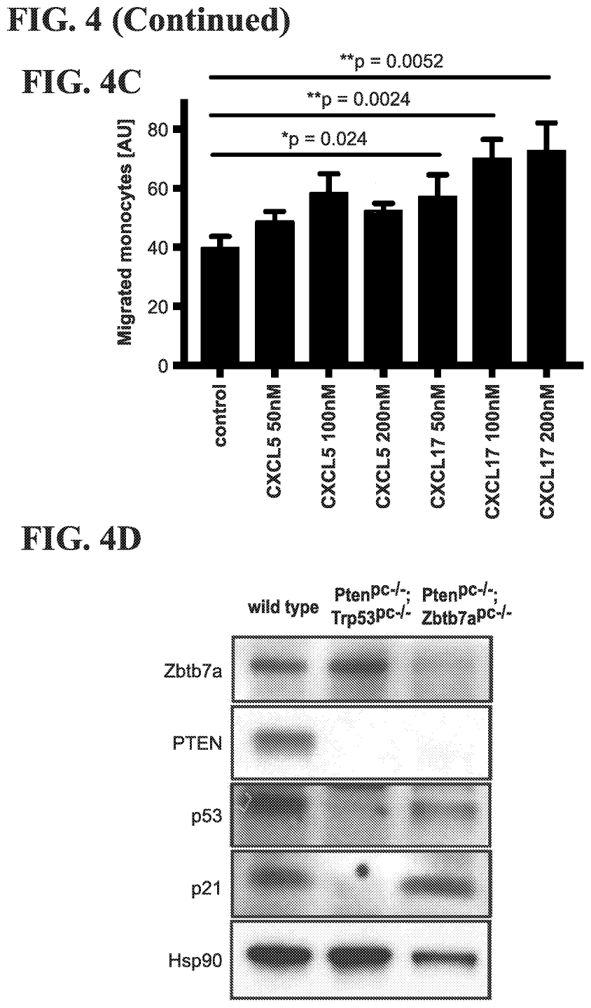

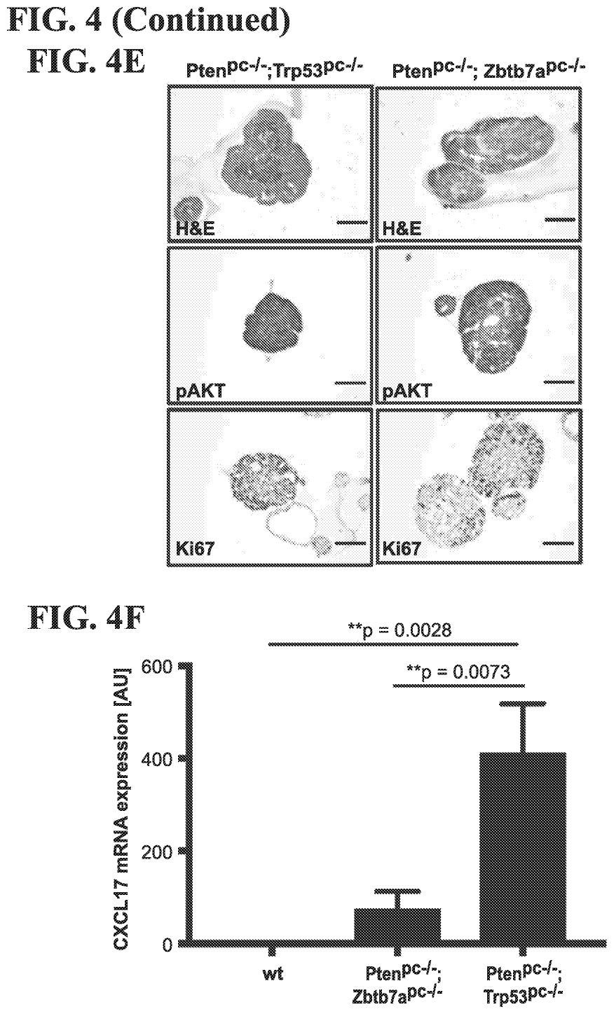

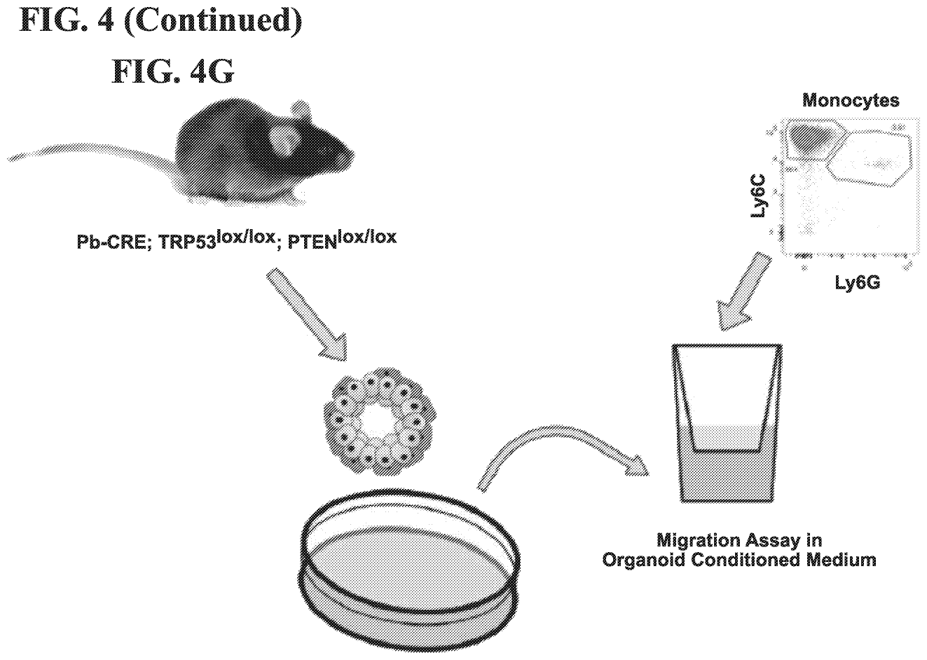

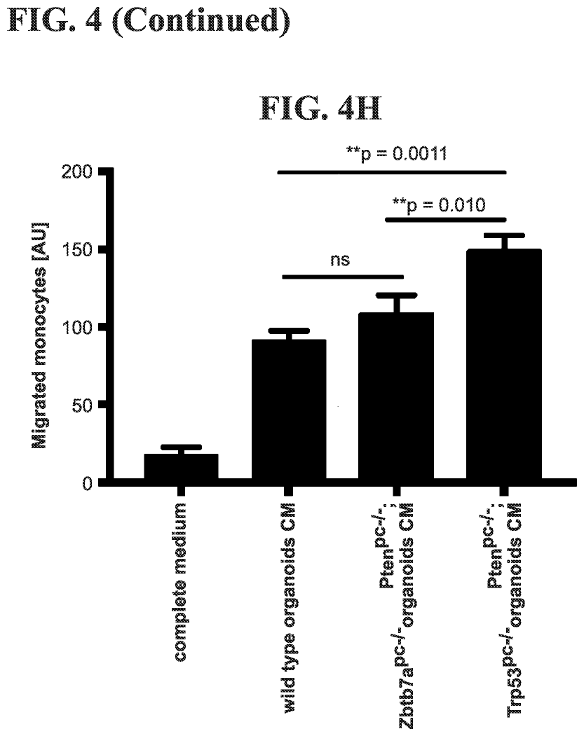

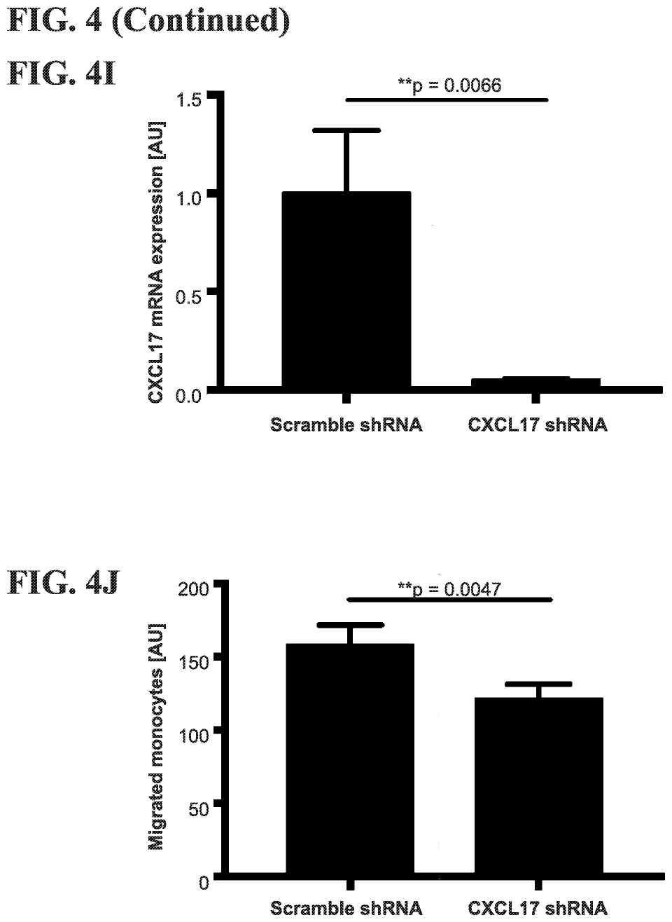

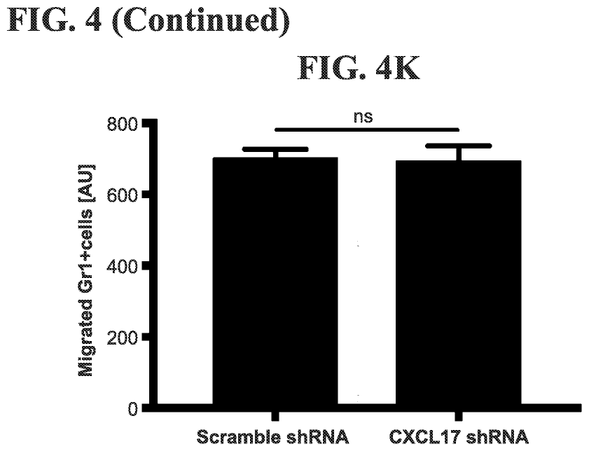

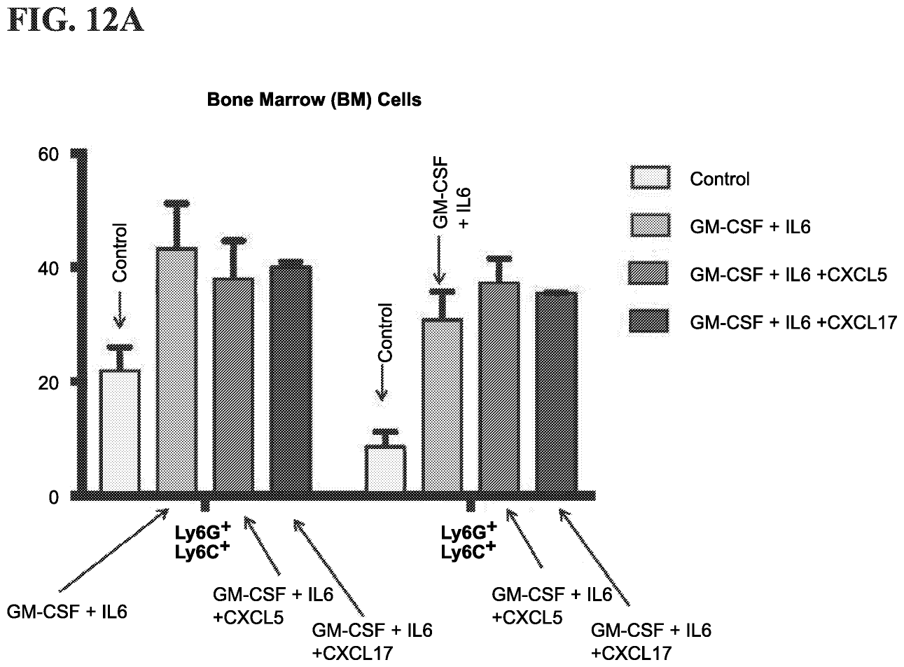

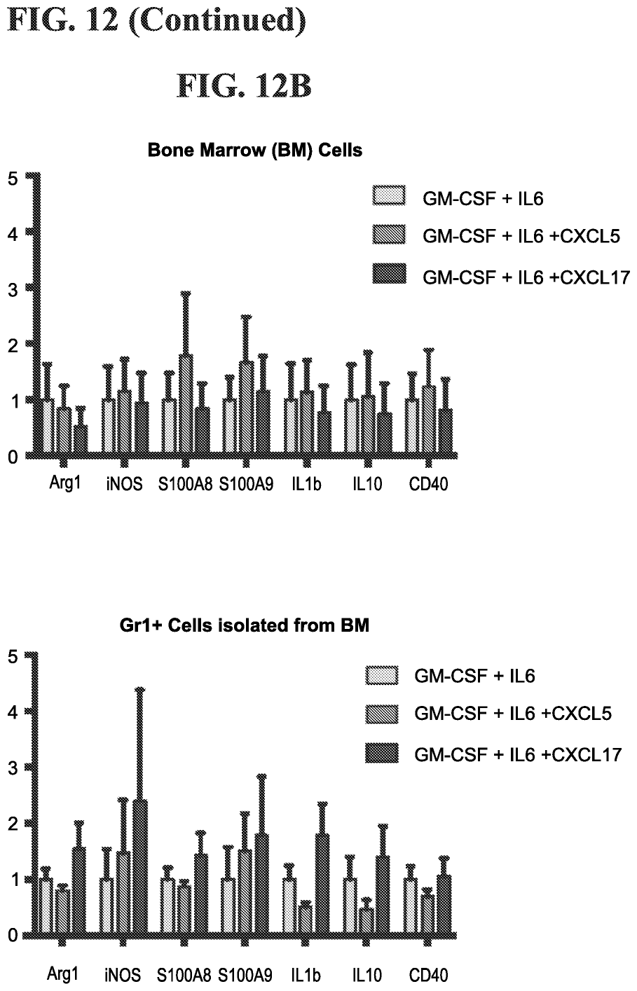

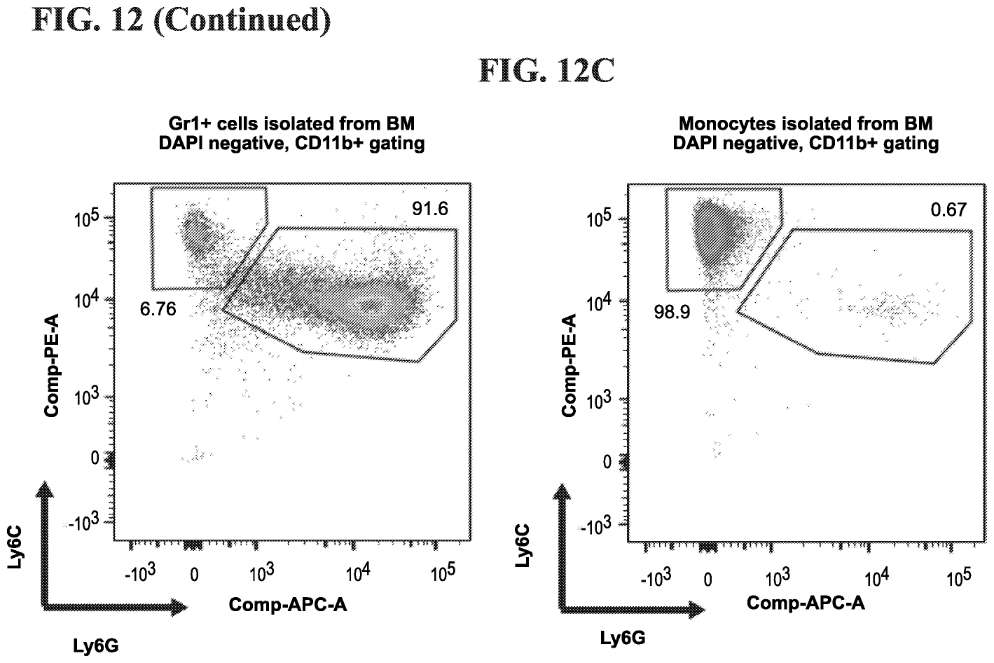

Immature myeloid cells that reside in the bone marrow can be cultured and induced to acquire phenotypic features of MDSC upon addition of GM-CSF and interleukin-6 (IL6) to the culture medium. To analyze the potential role of CXCL5 and CXCL17 in shaping the TME, whole bone marrow (BM) mouse cells were first cultured, and Gr1+ cells isolated from the BM with IL6 and GM-CSF alone or in the presence of either CXCL5 or CXCL17. After 4 days of culture the experiments of this did not observe any significant difference in the expression of the Ly6C and Ly6G markers or in the expression profile of the genes tested (FIG. 4A, FIG. 12A, and FIG. 12B), suggesting that these two chemokines are not major factors in the determination of immature myeloid cells phenotype. Nevertheless, in accordance with previous studies (Wang, G. et al. Cancer Discov 6, 80-95 (2016); Pisabarro, M. T. et al. The Journal of Immunology 176, 2069-2073 (2006)), the experiments of this example were able to validate the functions of CXCL5 and CXCL17 as chemoattractants for PMN and monocytic cells, respectively. A transwell migration assay was performed by using recombinant proteins and either Gr1+ cells (which are mostly Ly6G+/Ly6C^{int} PMN cells) or monocytes isolated from the bone marrow of healthy mice (FIG. 11B). CXCL17 showed a concentration-dependent effect only on monocytes (FIG. 4B, FIG. 4C).

Since there is currently no reliable anti-CXCL17 antibody available for use in vivo, it was sought to further assess the role of CXCL17 in Pten^{pc-/-}; Trp53^{pc-/-} tumors by establishing organoid cultures. Prostate cells were isolated from 3 months old Pten^{pc-/-}; Trp53^{pc-/-}, Pten^{pc-/-}; Zbtb7a^{pc-/-}, and wild type mice and were propagated in vitro by using a recently published 3D culture method (Karthaus, W. R. et al. Cell 159, 163-175 (2014); Drost, J. et al. Nat Protoc 11, 347-358 (2016)). Western blot analysis confirmed that the genetically targeted tumor suppressor genes were almost completely absent (FIG. 4D). Furthermore, IHC showed Pten^{pc-/-}; Trp53^{pc-/-}, Pten^{pc-/-}; Zbtb7a^{pc-/-} organoids with a histological pattern similar to the mouse model of origin, as well as elevated levels of both pAKT and Ki67 (FIG. 4E). Importantly, CXCL17 expression was strikingly higher in the Pten^{pc-/-}; Trp53^{pc-/-} organoids, when compared to Pten^{pc-/-}; Zbtb7a^{pc-/-} and wild type organoids (FIG. 4F).

To validate the 3D culture approach as a suitable tool for tumor-TME interaction studies, a transwell migration assay was performed using organoid conditioned medium (CM) and monocytes isolated from the bone marrow of 3 months old mice (FIG. 4G and FIG. 12C). Notably, in line with what was observed in prostate cancer mouse models, the migration of monocytic cells was enhanced in CM from Pten^{pc-/-}; Trp53^{pc-/-} organoids when compared to CM from Pten^{pc-/-}; Zbtb7a^{pc-/-} and wild type organoids (FIG. 4H). To examine whether CXCL17 is important in the recruitment of monocytes by Pten^{pc-/-}; Trp53^{pc-/-} tumors, next, Pten^{pc-/-}; Trp53^{pc-/-} organoids were generated stably expressing a shRNA against CXCL17 (FIG. 4I). CXCL17 knockdown reduced the migration of monocytes to a degree that was similar to what was observed for Pten^{pc-/-}; Zbtb7a^{pc-/-} organoids (FIG. 4J and FIG. 4H), but had no effect on the migration of Gr1+ cells isolated from the BM (FIG. 4K). Collectively, the results of this example support the idea that CXCL17 could act as a chemoattractant for Mo-MDSCs in the Pten^{pc-/-}; Trp53^{pc-/-} tumor model.

### Example 5: Selective Blockade of Gr-1+/CD11b+ Cells in Pten^{pc-/-}; Zbtb7a^{pc-/-} and Pten^{pc-/-}; Trp53^{pc-/-} Impact Tumorigenesis

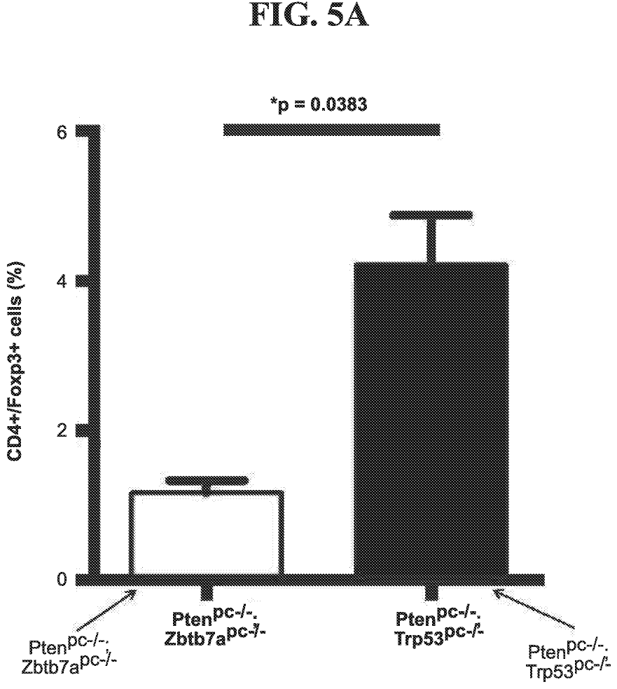

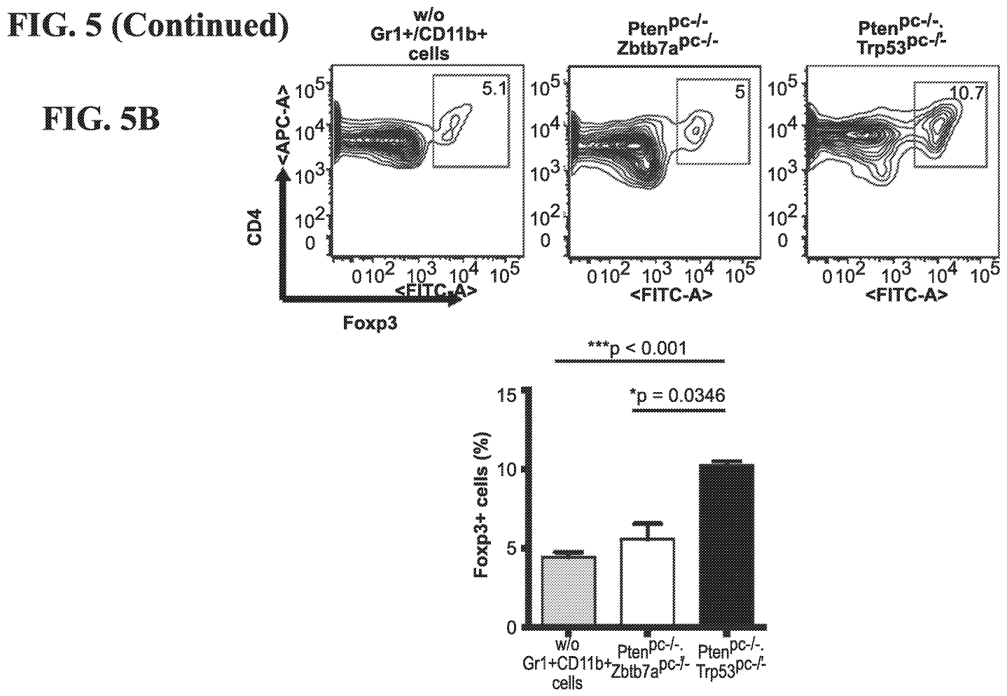

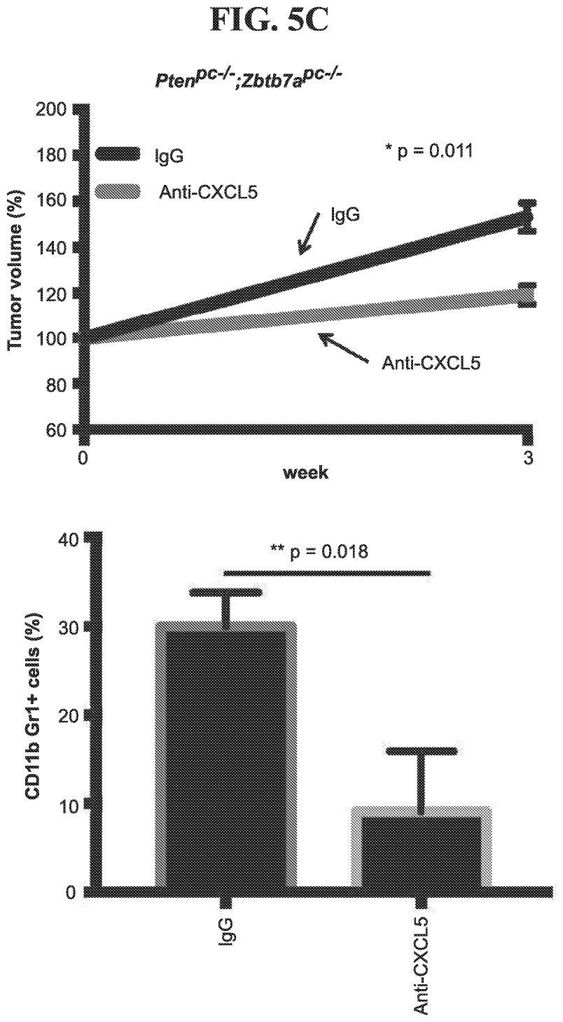

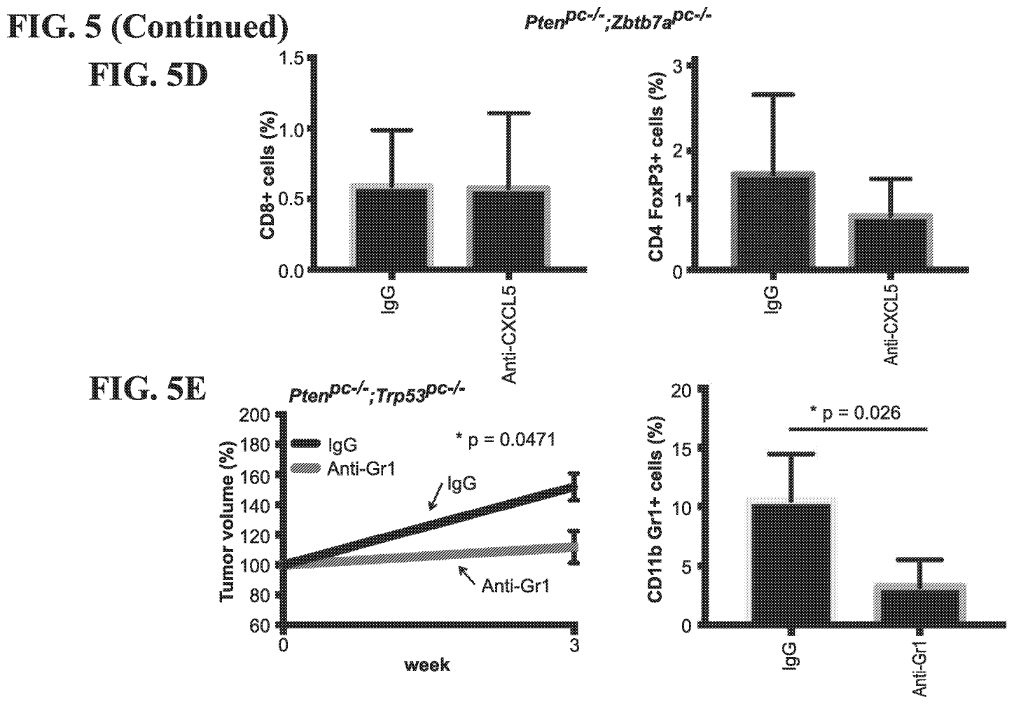

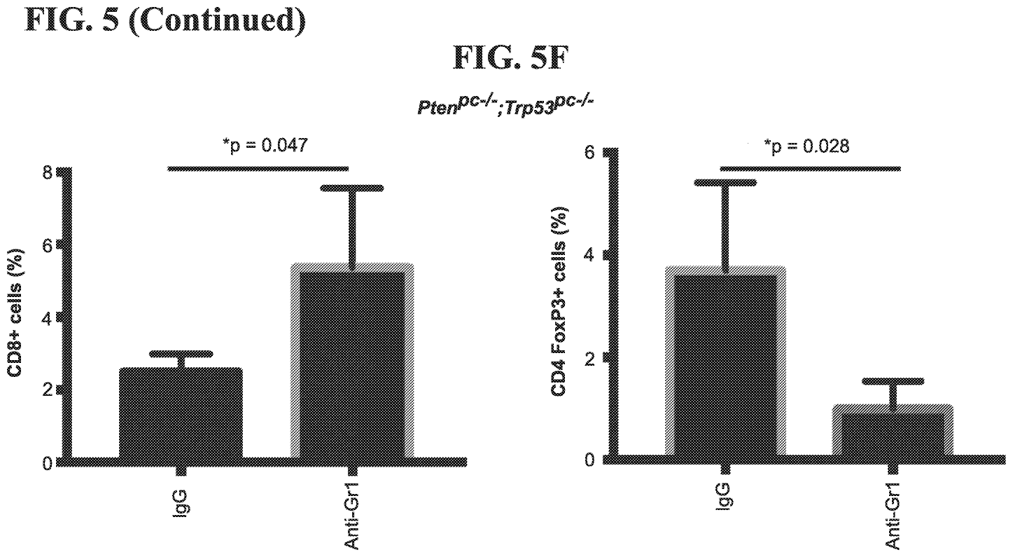

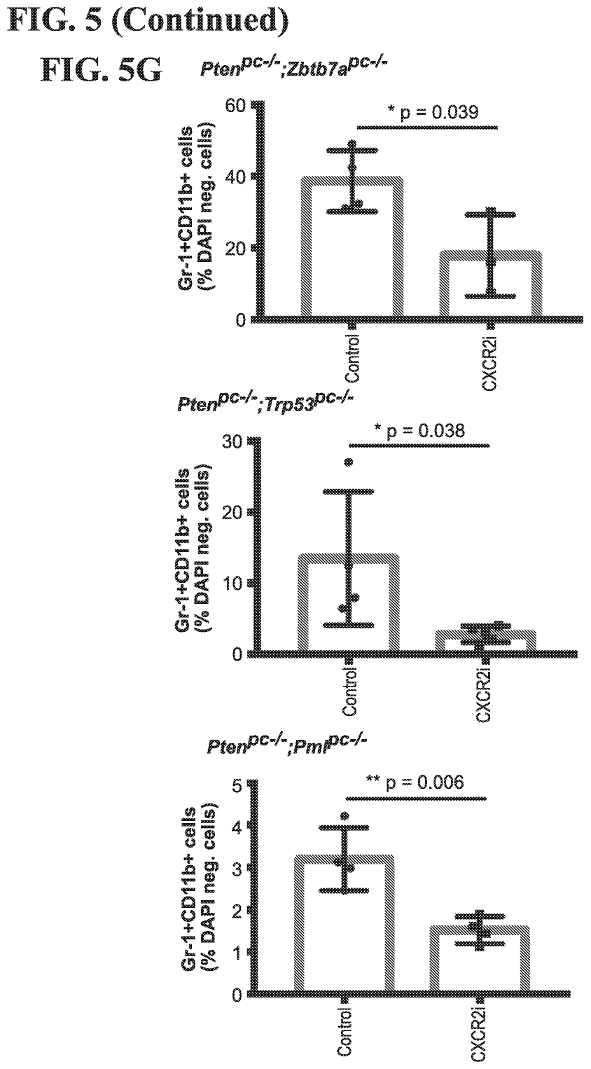

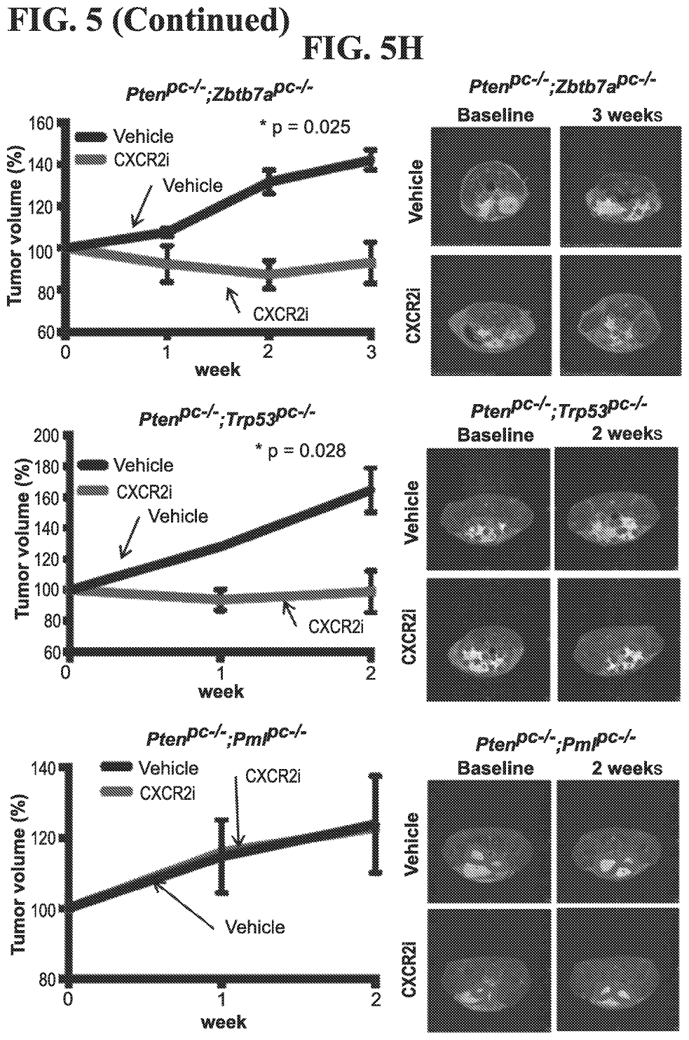

Gr-1+/CD11b+ cells are often implicated in tumor progression; however, the full impact they have on cancer cells is still being actively investigated. Additionally, studies regarding the contributions of Gr-1+/CD11b+ cells to tumor growth and metastasis show a context-dependent function, and are in some cases contradictory (Colombo, M. P. et al. Journal of Experimental Medicine 173, 889-897 (1991); Pekarek, L. A. et al. Journal of Experimental Medicine 181, 435-440 (1995); Mittendorf, E. A. et al. Cancer Res. 72, 3153-3162 (2012); Cools-Lartigue, J. et al. J. Clin. Invest. 123, 3446-3458 (2013); Granot, Z. et al. Cancer Cell 20, 300-314 (2011); Haverkamp, J. M. et al. Immunity 41, 947-959 (2014)). Based on the gene expression analysis in this example (FIG. 2B, FIG. 2C, and FIG. 2D), and on the presence of Treg cells in Pten^{pc-/-}; Trp53^{pc-/-} tumors at both 3 and 6 months of age (FIG. 5A and FIG. 8E), ex vivo co-culture of CD4+ T cells with Gr1+/CD11b+ cells sorted from either Pten^{pc-/-}; Trp53^{pc-/-} or Pten^{pc-/-}; Zbtb7a^{pc-/-} tumors was performed. Notably, only Gr1+/CD11b+ cells sorted from Pten^{pc-/-}; Trp53^{pc-/-} mice were able to significantly induce the expansion of Treg cells (FIG. 5b). Therefore, in accordance with the recently published recommendation for myeloid derived suppressor cell (MDSC) nomenclature (Brandau, S. et al. Nature Communications 7, 1-10 (2016)), at 3 months of age the Pten^{pc-/-}; Trp53^{pc-/-} TME is characterized by the presence of Mo-MDSCs whereas Pten^{pc-/-}; Zbtb7a^{pc-/-} tumors are infiltrated by PMN-MDSC-like cells (PMN-MDSC-LC).

Next, to assess the role of Gr-1+/CD11b+ cells in the Pten^{pc-/-}; Trp53^{pc-/-} mouse model, an anti-Gr1 monoclonal antibody was used for the depletion of both Ly6G+ and Ly6C+ cells. After 3 weeks of treatment, a significant volume reduction of the tumors was observed and confirmed a significant depletion of intra-tumoral Gr-1+/CD11b+ cells (FIG. 5E and Table 1). Anti-Gr1 treated Pten^{pc-/-}; Trp53^{pc-/-} mice displayed an altered immune landscape characterized by a decrease in Treg cells associated with an increase in CD8+ T cells (FIG. 5F). This analysis validated the Pten^{pc-/-}; Trp53^{pc-/-} myeloid infiltrate as Mo-MDSCs.

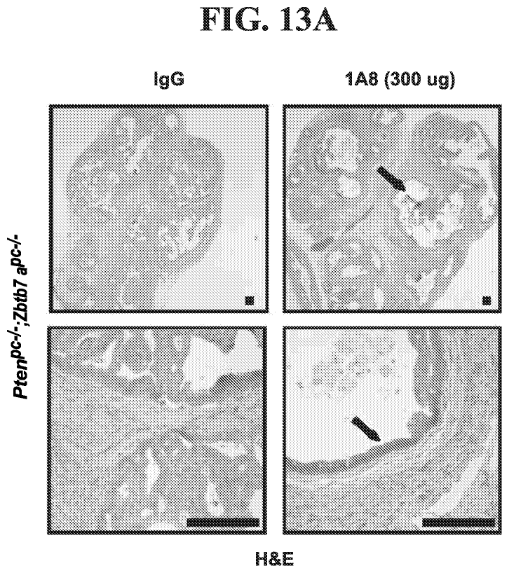

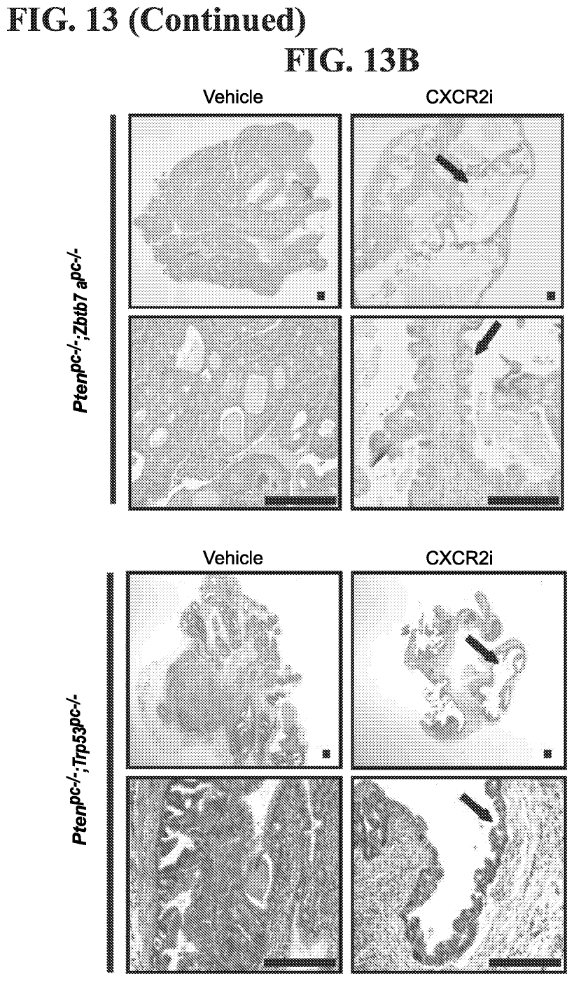

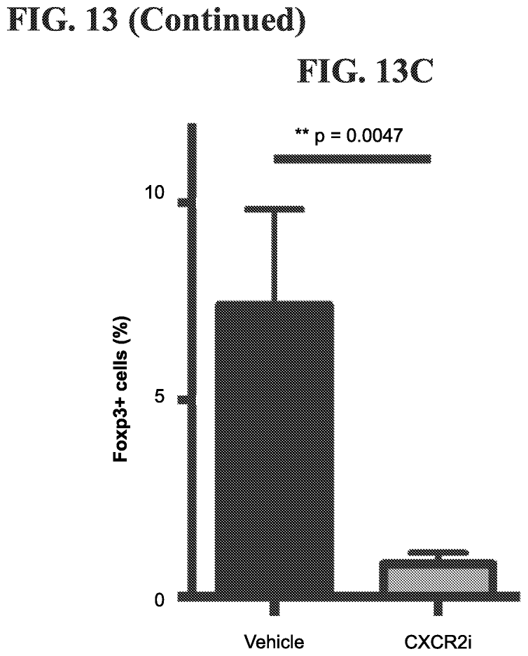

Finally, an aim of the experiments of this example sought to determine tumor growth rates upon CXCR2 antagonist SB225002 treatment, which is known to inhibit Gr-1+/CD11b+ cell attraction. Indeed, CXCR2 inhibition led to a decrease of Gr-1+/CD11b+ cells in all the models tested (FIG. 5G). To assess the effect of Gr-1+/CD11b+ cell depletion on tumor growth, the tumor volume of anterior lobe lesions was quantified on a weekly basis by MRI after CXCR2 inhibitor treatment. It was observed that CXCR2 inhibition significantly suppressed the tumor growth of both Pten^{pc-/-}; Zbtb7a^{pc-/-} and Pten^{pc-/-}; Trp53^{pc-/-} tumors but not the tumor growth of the “immune-depleted”Pten^{pc-/-}; Pml^{pc-/-} tumors (FIG. 5H and Table 1). Subsequent histological analysis in Pten^{pc-/-}; Zbtb7a^{pc-/-} and Pten^{pc-/-}; Trp53^{pc-/-} mice demonstrated that, while vehicle treated tumors displayed large tumor areas containing PIN lesions and complex glandular structures, the CXCR2 inhibitor treated mice displayed prostate glands with significantly diminished tumor involvement and large tumor cysts (FIG. 13B). Moreover, Pten^{pc-/-}; Trp53^{pc-/-} prostate tumors showed less Treg cells after treatment with SB225002 (FIG. 13C). Altogether, these data reveal that Gr-1+/CD11b+ cells in the Pten^{pc-/-}; Zbtb7a^{pc-/-} tumors and in the Pten^{pc-/-}; Trp53^{pc-/-} tumors, but not in the Pten^{pc-/-}; Pml^{pc-/-} tumors, exert a critical role in tumor progression and maintenance.

### Example 6: Gr-1+/CD11b+ Cells in Pten^{pc-/-}; Zbtb7a^{pc-/-} Promote Tumor Progression by Impacting the NFκB Signaling Pathway

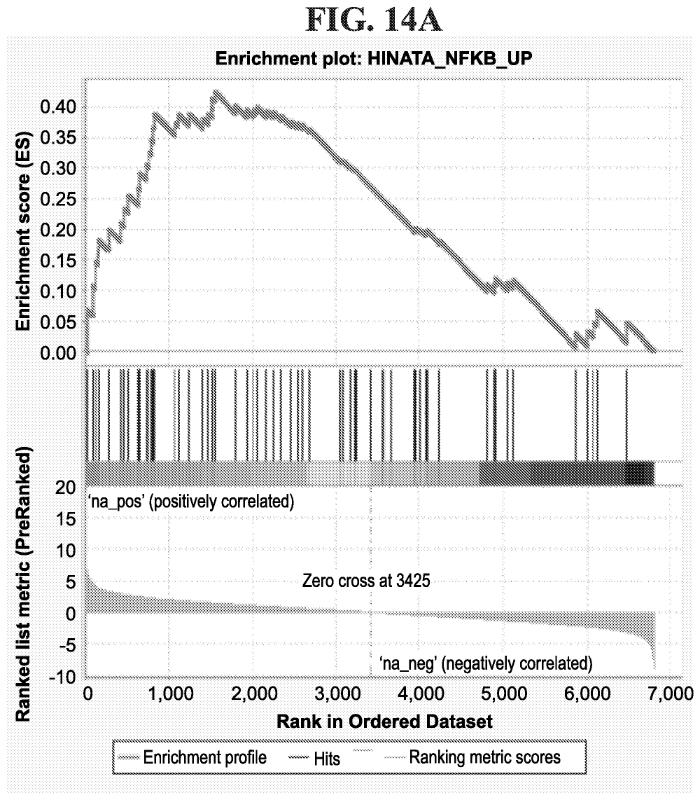

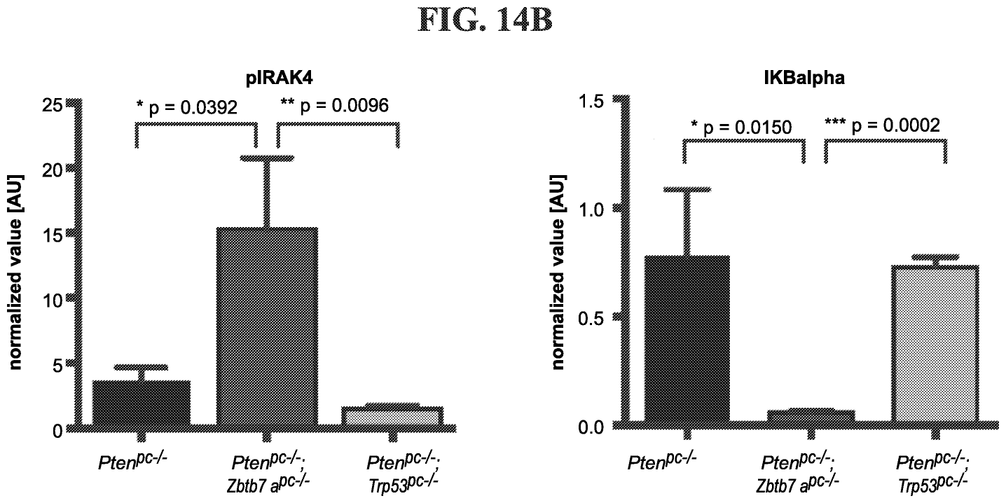

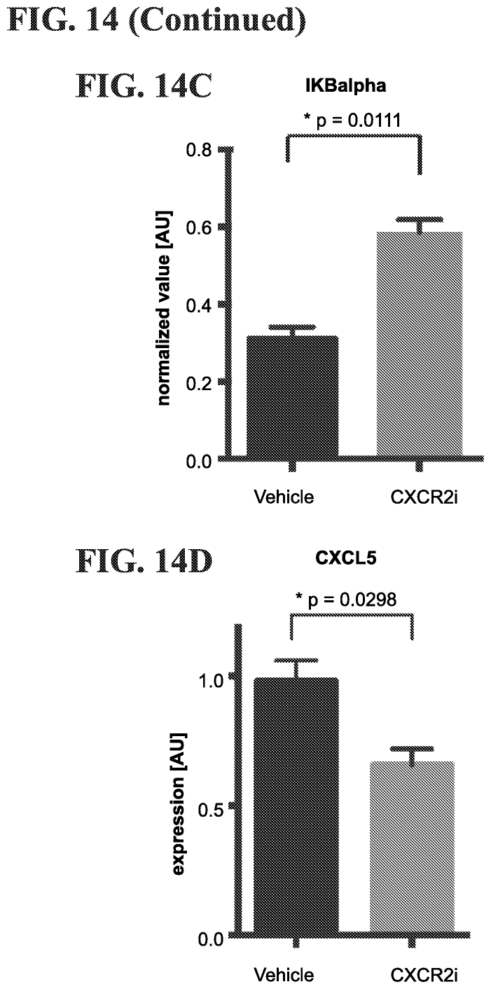

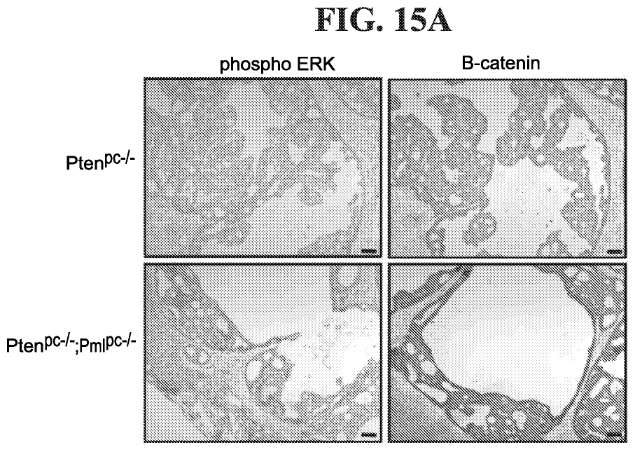

Similar to Pten^{pc-/-}; Zbtb7a^{pc-/-} tumors, PMN cells have recently been observed in a different mouse model of prostate cancer, the Pten^{pc-/-}; Smad4^{pc-/-} mouse model (Wang, G. et al. Cancer Discov 6, 80-95 (2016)). However, in that specific case, as well as in other tumor types (Kumar, V. et al. Trends in Immunology 37, 208-220 (2016)), the PMN infiltrate displayed immunosuppressive activity. Therefore, the experiments of this example further investigated the mechanism by which PMN-MDSC-LCs promote Pten^{pc-/-}; Zbtb7a^{pc-/-} tumor growth. As shown in FIG. 2, these cells expressed high levels of S100A8, S100A9 and IL1b. S100A9 was previously implicated in tumor progression through the upregulation of several pro-tumorigenic signaling pathways, including NFκB signaling through the activation of the RAGF/TLR4 receptors (Markowitz, J. et al. Biochim. Biophys. Acta 1835, 100-109 (2013)). Similarly, IL1b is known as a regulator of inflammatory responses and a pro-tumorigenic cytokine. It also equally activates NFκB signaling through its type 1 receptor. In line with this, gene set enrichment analysis of microarray data obtained from 3 month old Pten^{pc-/-} and Pten^{pc-/-}; Zbtb7a^{pc-/-} tumors shows an enrichment for NFκB target genes, specifically in Pten^{pc-/-}; Zbtb7a^{pc-/-} tumors (FIG. 14A), that could be validated by western blot analysis of increased pIRAK4 and decreased IκBα expression in Pten^{pc-/-}; Zbtb7a^{pc-/-}, compared to Pten^{pc-/-} and Pten^{pc-/-}; Trp53^{pc-/-} tumors (FIG. 14B). Conversely, western blot analysis of Pten^{pc-/-}; Zbtb7a^{pc-/-} tumors treated with the CXCR2 antagonist SB225002 showed increased IκBα protein levels (FIG. 14C) as well as elevated expression of CXCL5, a known NFκB target gene (FIG. 14D). This result indicates a negative regulation of NFκB signaling after inhibition of Gr-1+/CD11b+ cell recruitment, and thereby links tumor promotive NFκB activation with Gr-1+/CD11b+ cell activity. These data further indicate that Gr-1+/CD11b+ cells in the TME of Pten^{pc-/-}; Zbtb7a^{pc-/-} tumors display a specific phenotype and tumor promotive activity when compared to Pten^{pc-/-} and Pten^{pc-/-}; Trp53^{pc-/-} tumors.

### Example 7: Validation of the Association Between Tumor Genetic Make-Ups and Differential Immune-Infiltrates in Human Samples

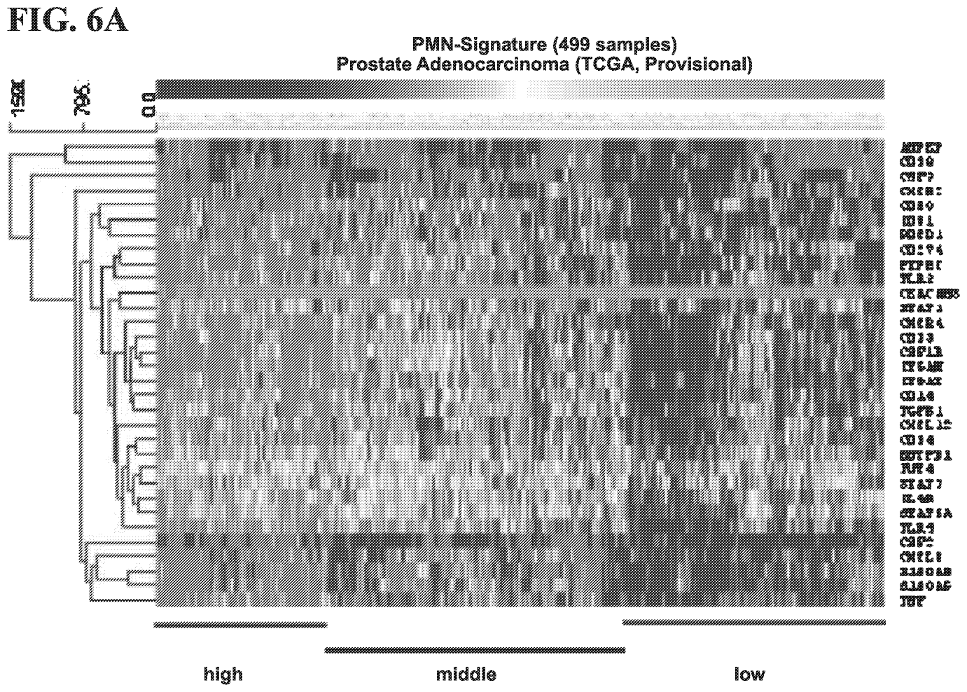

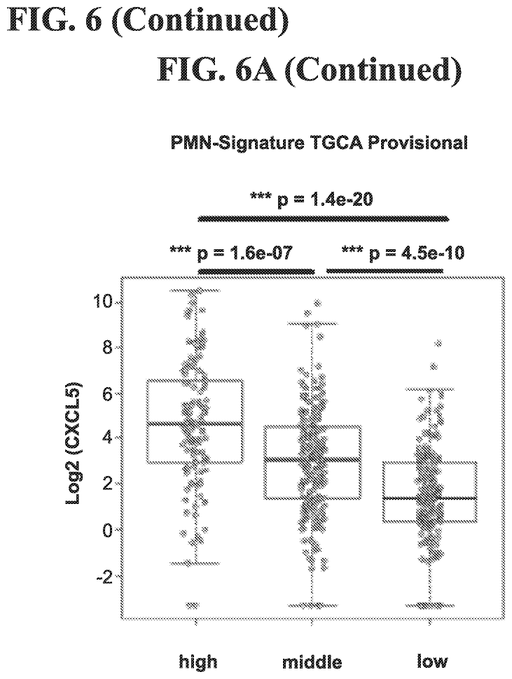

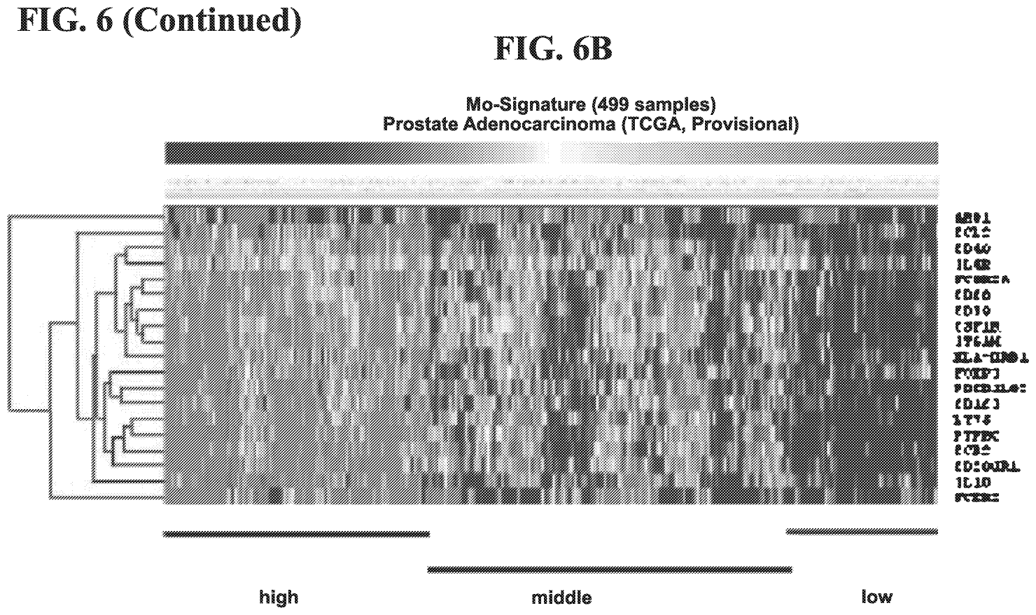

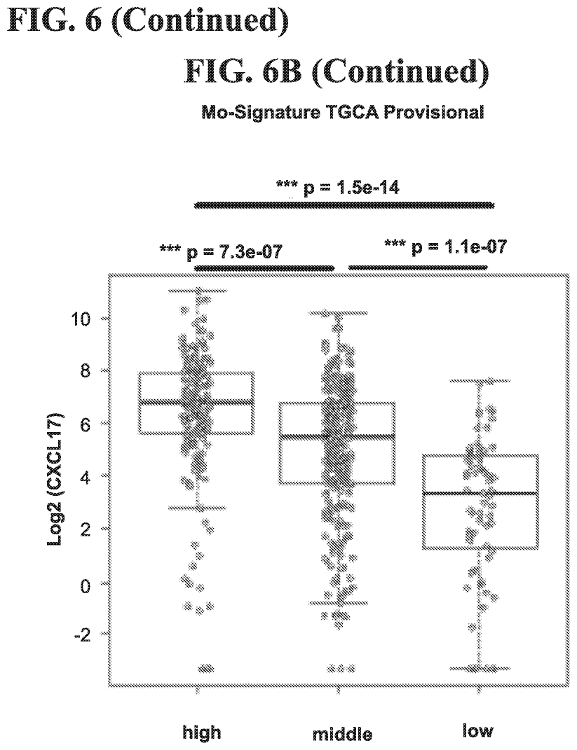

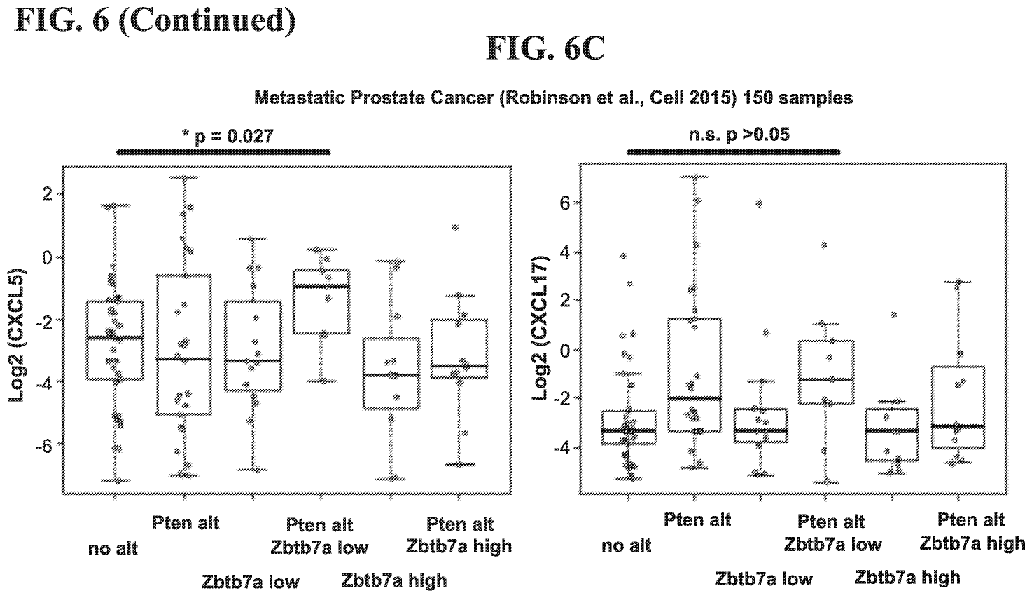

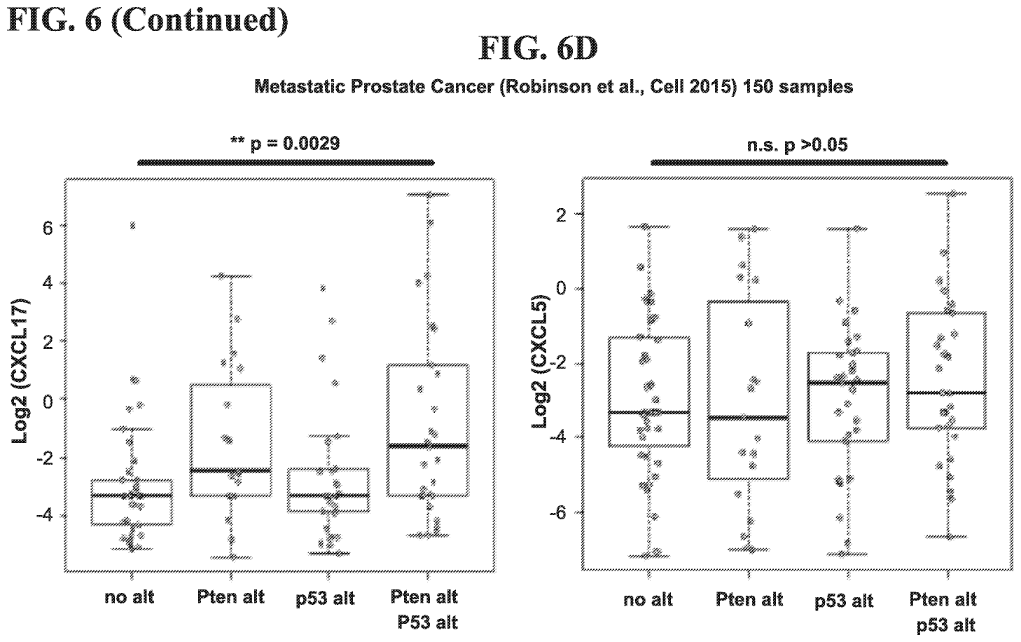

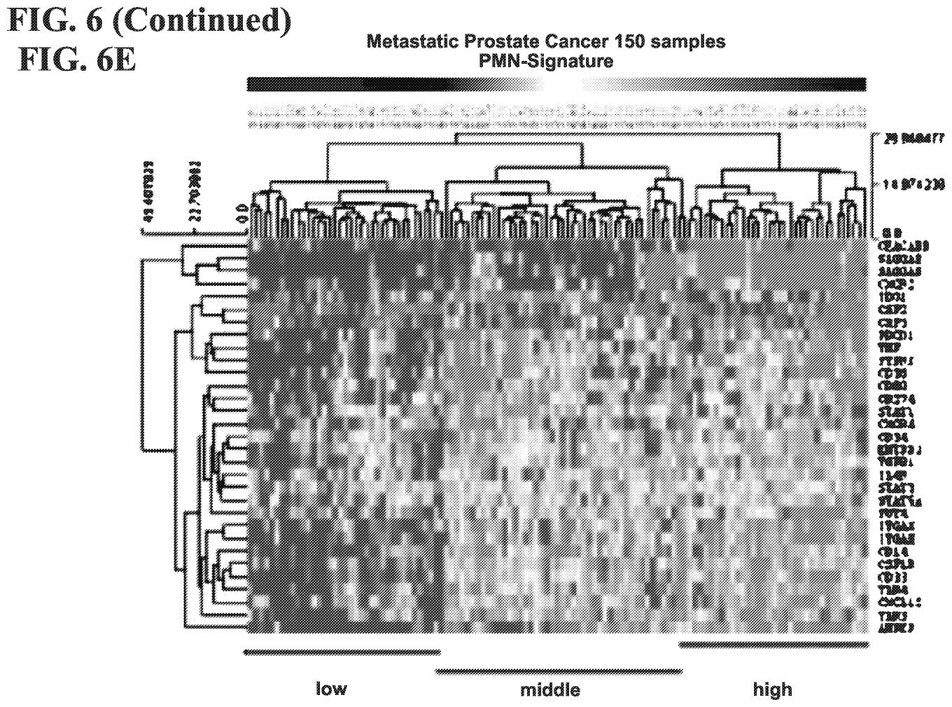

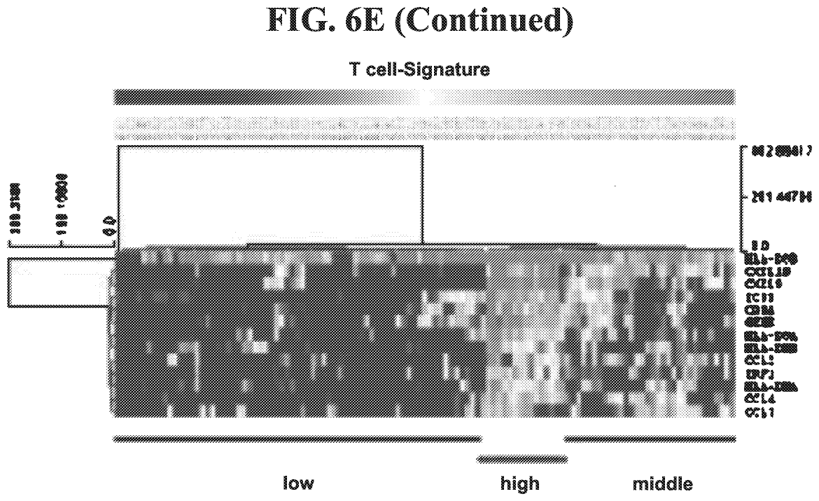

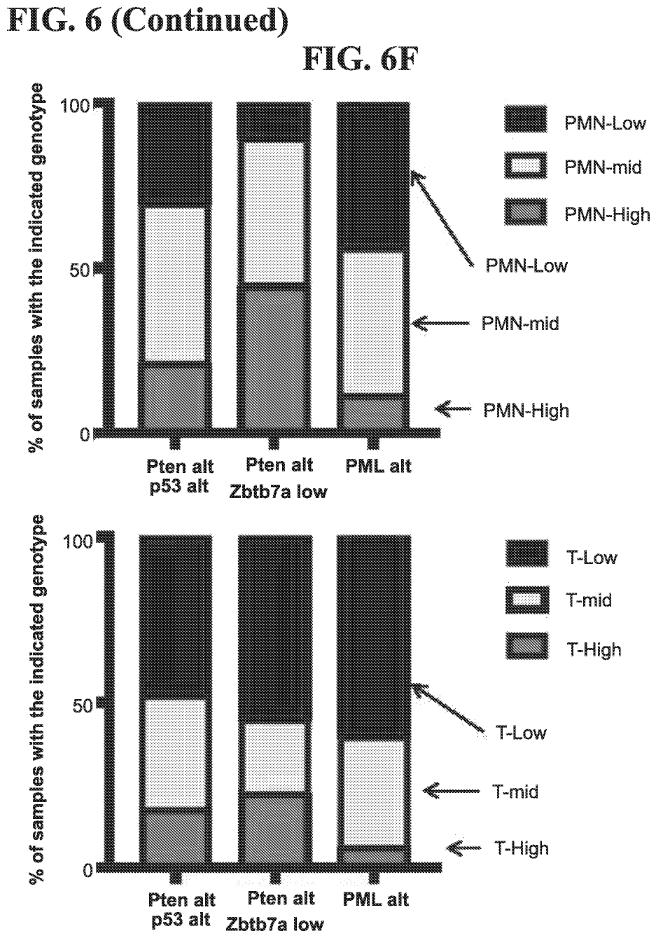

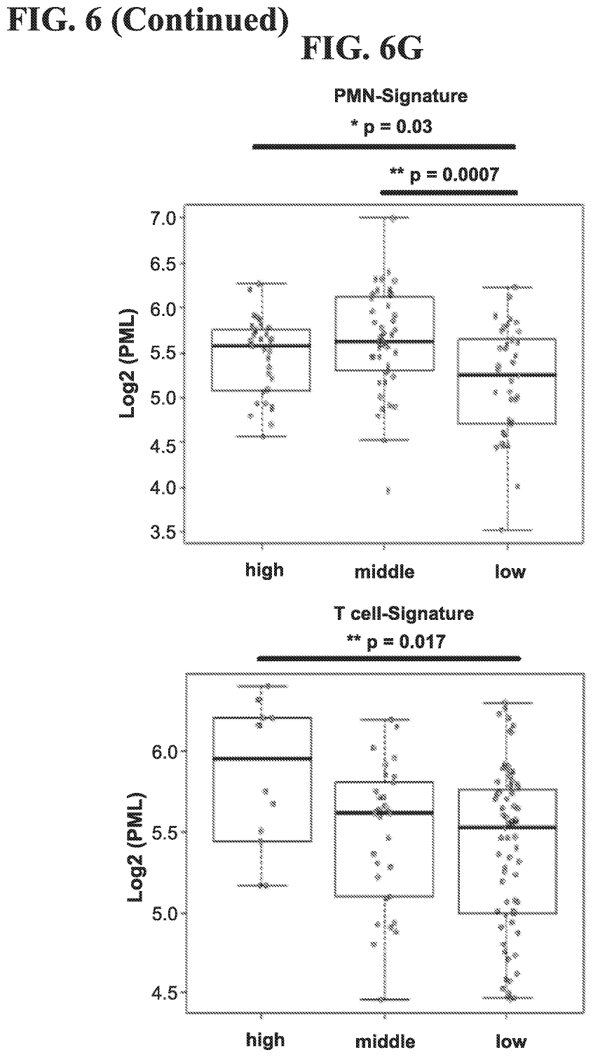

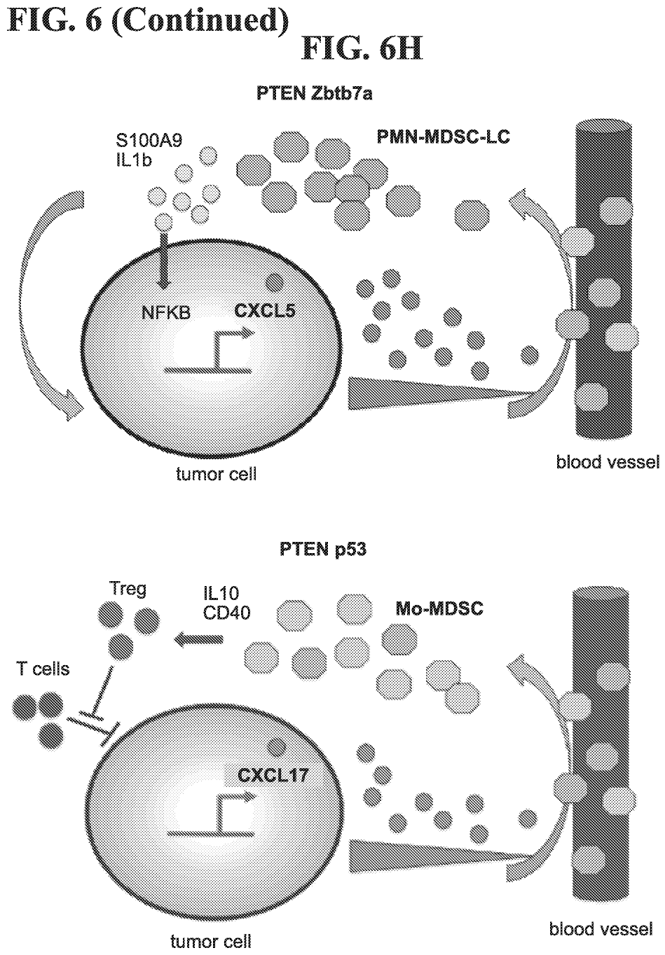

Gene expression signature analysis has been shown to be an effective method to characterize the TME and can have a profound prognostic potential (Gentles, A. J. et al. Nat. Med. 1-12 (2015)). The experiments of this example took advantage of such approach to validate, in human samples, the association between CXCL5/17 and tumor-associated immune cells. To this end, the experiments of this example interrogated the 499 samples of “The Cancer Genome Atlas” (TGCA) provisional prostate adenocarcinoma dataset using a gene signature for PMN cells (PMN-Signature) and a gene signature for monocytic MDSCs and M2-like macrophages (Mo-Signature) (Table 2). Table 2 below shows the gene signatures used for the analysis in FIG. 6.

TABLE 2

PMN-signature
CXCR4
CXCR2
ITGAM
ITGAX
ANPEP
CD14
FUT4
CD33
CD34
CD38
ENTPD1
PTPRC
CEACAM8
CD80
CSF1R
IL4R
CSF3
CSF2
CXCL8
TNF
CXCL12
CSF1R
S100A8
S100A9
STAT1
STAT3
STAT5A
ARG1
NOS2
CD274
TLR3
TLR4
TGFB1
IL10
IDO1
PDCD1
Mo-MDSC/M2
Macrophages-signature
CD14
CD124
CD45
CD11B
CD33
ARG1
IL10
CD40
CD32
CD163
CD23
CD200R
PD-L2
CD68
CD115
HLA-DR
CD205
CCR2
CCL2
FOXP3
T-Cell-Signature
CD8A
CCL2
CCL3
CCL4
CXCL9
CXCL10
ICOS
GZMK
IRF1
HLA-DMA
HLA-DMB
HLADOA
HLA-DOB

The PMN gene signature was generated by modifying a recently published 39-gene MDSC signature (Wang, G. et al. Cancer Discov 6, 80-95 (2016)). This signature clustered the TGCA samples into three groups: PMN-high, PMN-mid and PMN-low. The Mo-signature was created from literature mining (Ugel, S. et al. Journal of Clinical Investigation 125, 3365-3376 (2015)), and was used to categorize the TGCA provisional prostate adenocarcinoma dataset into the three groups Mo-high, Mo-mid and Mo-low (FIG. 6B). CXCL17 showed higher expression levels in the Mo-high group, which directly correlated with the findings of this disclosure (FIG. 6A, FIG. 6B).

The concomitant deregulation of the tumor suppressor genes PTEN and TP53 is a common characteristic of advanced human prostate cancer. Similarly, it has been recently shown that in patients with altered PTEN, low levels of Zbtb7a are associated with aggressive castration-resistant prostate cancer (Lunardi, A. et al. Nat. Genet. 45, 747-755 (2013)). The experiments of this example therefore explored the possible link between these genetic make-ups and the expression of CXCL5 and CXCL17 in human prostate cancer. To this end, the experiments of this example first examined a publicly available dataset of metastatic prostate cancer (Robinson et al. (n=150) (Robinson, D. et al. Cell 161, 1215-1228 (2015)). In line with the data obtained from the Pten^{pc-/-}; Zbtb7a^{pc-/-} model, the cohort with PTEN altered (deleted or mutated) and ZBTB7A low status showed higher expression of CXCL5 (FIG. 6C), but not that of CXCL17. To investigate the human relevance of CXCL17 expression in relation to PTEN and p53 loss, the experiments of this example focused the analysis on four patient cohorts: wild type PTEN and TP53 (no alt), PTEN homozygous alteration (PTEN alt), TP53 homozygous alteration (p53 alt), and concomitant PTEN and TP53 deficiency based on homozygous deletion or mutations (PTEN alt; p53 alt). The results were once again consistent with the findings of this disclosure in the mouse models. The cohort of PTEN alt; p53 alt patients showed the highest expression of CXCL17, whereas CXCL5 expression did not differ significantly among the different groups (FIG. 6D).

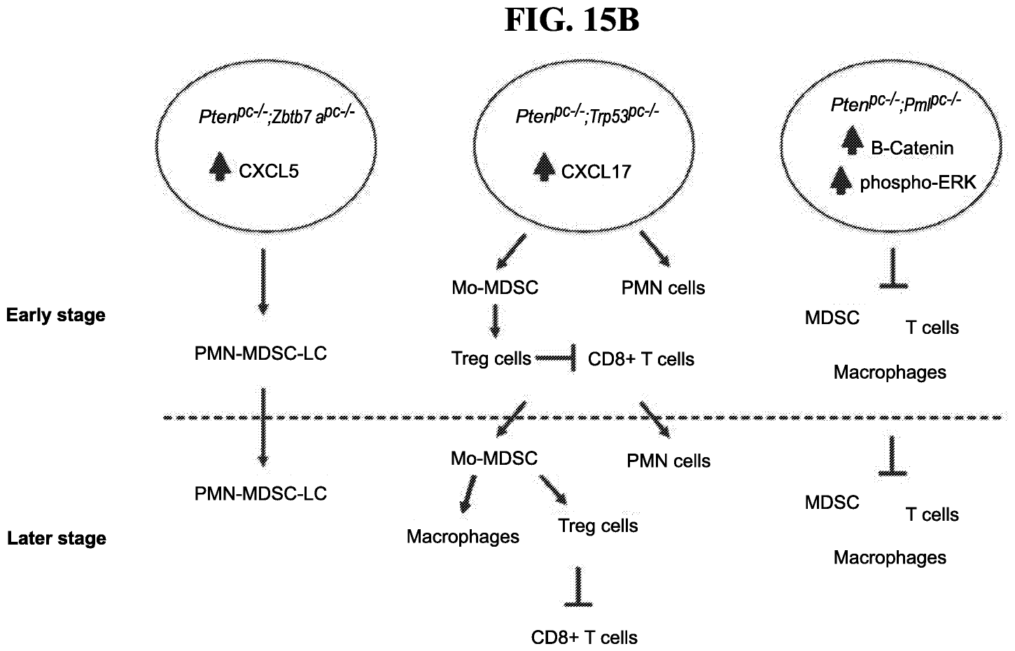

Next, the experiments of this example focused the analysis on prostate cancer genetics vis a vis different immune landscapes. The experiments of this example used the aforementioned PMN-signature and a previously published T cell signature (Spranger, S. et al. Nature 523, 231-235 (2015)) (Table 2) to categorize the 150 metastatic prostate cancer samples of the Robinson dataset. The sequencing profiles were grouped into the high-, mid- and low-infiltrate clusters (FIG. 6E) and it was analyzed how patients with genetics similar to the mouse models investigated in the study of this disclosure were distributed among the different groups. Remarkably, only 1 out 9 patients (11.1.%) with altered PTEN and Zbtb7a showed low PMN infiltrate and only 2 out of 18 PML deleted patients (11.1%) clustered into the PMN high type. To further confirm the link between loss of PML and a non-inflamed “cold” TME, only 1 out of 18 samples (5.6%) displayed high expression of the T cell-signature (FIG. 6F). Additionally, PML expression levels were analyzed in the different clusters. Significantly lower levels of PML expression were observed in the samples with less PMN and T-cell infiltrate (FIG. 6G). The association data that include the PML genetic status and expression levels are particularly relevant as recent extensive cancer-immune profile studies have resulted in the identification of an immune-desert phenotype characterized by a “cold”, non-inflamed, tumor microenvironment (Chen, D. S. et al. Nature 541, 321-330 (2017)), similar to the one that has been described in this disclosure in the Pten^{pc-/-}; Pml^{pc-/-} prostate cancer model. This is of clinical relevance because this phenotype appears to be resistant to anti-PD-L1/PD1 therapy. Two major tumor oncogenic pathways have been directly linked to the immune-desert phenotype: the β-catenin and the MAPK signaling pathways (Spranger, S. et al. Nature 523, 231-235 (2015); Seliger, B. et al. Exp. Hematol. 24, 1275-1279 (1996); Seliger, B. et al. Eur. J. Immunol. 28, 122-133 (1998); Atkins, D. et al. Int. J. Cancer 109, 265-273 (2004); Bradley, S. D. et al. Cancer Immunology Research 3, 602-609 (2015)). Notably, Pten^{pc-/-}; Pml^{pc-/-} prostate cancers at 3 months of age showed upregulation of both β-catenin and phospho-ERK (FIG. 15). In summary, the data of the experiments of this example, obtained from GEMMs, correlates with what is observed in human clinical cancer samples, highlighting the relevance of the approach of this disclosure towards patient stratification.

Diverse immune cell types can infiltrate and interact with solid and liquid tumors and have an impact on virtually every therapeutic approach by multiple mechanisms that appear to be extremely context specific. The experiments of this disclosure hypothesized that distinct genomic alterations may shape the TME in a genotype-specific manner based on distinct chemokine pools resulting from specific transcriptional and signaling programs. As demonstrated herein, the diverse genetic background of prostate cancer can directly, and cell autonomously, determine the differential infiltration and composition of immune cells in the TME (FIG. 6H, FIG. 15B), as well as the suppression of the immune cell infiltration.

As disclosed herein, the recruitment of distinct Gr-1+/CD11b+ cells to prostate tumors is directly regulated by the genetic make-up in mouse models, as well as in human cancer. Specifically, the experiments of this disclosure have shown that loss of p53 leads to upregulated expression of the known Gr-1+/CD11b cell attractant CXCL17. In line with these findings, human prostate cancer specimens that are deficient of p53 and PTEN show a significantly higher expression of CXCL17.

The experiments of the present disclosure further show that tumor associated Gr-1+/CD11b+ cells exhibit a tumor-promoting phenotype in both Pten^{pc-/-}; Zbtb7a^{pc-/-} as well as Pten^{pc-/-}; p53′ that can be blocked pharmacologically. However, the mechanisms of tumor promotion differ dramatically. In Pten^{pc-/-}; Zbtb7a^{pc-/-} tumors, infiltrating Gr-1+/CD11b+ cells exhibit a PMN-MDSC-LC phenotype that promotes tumor progression directly by impacting the NFκB signaling pathway through the secretion of S100A9 and IL1b. (FIG. 6H)

In addition to the “tumor-promoting” immune landscape of the Pten^{pc-/-}; Zbtb7a^{pc-/-} model and the “immuno-suppressive” phenotype of the Pten^{pc-/-}; p53^{pc-/-} tumors, described herein is a third scenario: “the immune-desert” phenotype of Pten^{pc-/-}; Pml^{pc-/-} prostate cancer. Albeit aggressive and lethal, this model showed very limited intra-tumoral immune infiltrate when compared to the other models and to the control mice. Importantly, Pten^{pc-/-}; Pml^{pc-/-} mice did not respond to CXCR2i treatment. In keeping with these findings, and as a potential explanation for such phenotype, the loss of Pml has been recently associated with decreased cytokine production (Lunardi, A. et al. Genes & Cancer 2, 10-19 (2011)), and the upregulation of β-catenin and the activation of the MAPK pathway has been implicated in suppressing anti-tumor immunity (Spranger, S. et al. Nature 523, 231-235 (2015); Seliger, B. et al. Exp. Hematol. 24, 1275-1279 (1996); Seliger, B. et al. Eur. J. Immunol. 28, 122-133 (1998); Atkins, D. et al. Int. J. Cancer 109, 265-273 (2004); Bradley, S. D. et al. Cancer Immunology Research 3, 602-609 (2015)). The PTEN/PML model mimics the immune-desert “cold” phenotype observed in patients, which are known to be resistant to anti-PD-L1/PD-1 therapy (Chen, D. S. et al. Nature 541, 321-330 (2017)), and the PTEN/PML model of the present disclosure is currently the only prostate cancer preclinical model available for the investigation of this important cancer immune-phenotype.

As disclosed herein, the data regarding the qualitative difference of Gr-1+/CD11b+ cells attracted to prostate cancer may be especially relevant for tailoring immune therapies. By promoting T cells activation, immune checkpoint-targeting inhibitors have produced impressive results in multiple types of cancer, raising hope for a universal anti-tumoral approach. However, in recent clinical trials, the majority of prostate cancer patients showed resistance to such treatments (Small, E. J. et al. Clin. Cancer Res. 13, 1810-1815 (2007); Slovin, S. F. et al. Ann. Oncol. 24, 1813-1821 (2013); Kwon, E. D. et al. Lancet Oncol. 15, 700-712 (2014)). The findings of the present disclosure may be relevant for the stratification of a responsive patient population for combinatorial immunotherapy. For example, while the combination of immune checkpoint-targeting inhibitors with MDSC-depleting strategies may be extremely effective in patients with altered PTEN/TP53 and PTEN/SMAD4, it may not work as well in patients with altered PTEN/ZBTB7a or PTEN/PML.

Likewise, the unexpected findings of the present disclosure and Co-Clinical platform can significantly contribute to the ability to determine the mechanisms of action and the responder population for other compounds that did not show positive results in clinical trials with unselected patients. The S100A9 inhibitor Tasquinimod recently failed to show a clear survival benefit in a phase III clinical trial in prostate cancer (Williamson, S. C. et al. Drug Des Devel Ther 7, 167-174 (2013); Pili, R. et al. J. Clin. Oncol. 29, 4022-4028 (2011)). As clearly shown by the analysis of the present disclosure, such agents may interfere with only a certain subpopulation of tumors recruiting S100A9 secreting Gr-1+/CD11b+ cells. In addition, CXCR2 antagonists are currently under investigation in clinical trials, and could be found ineffective in tumors that do not recruit Gr-1+/CD11b+ cells.

Inter-patient cancer genetic heterogeneity is a major obstacle to successful cancer treatment and the data disclosed herein strongly suggests that next-generation clinical trials that are based on better patient stratification are essential to test the efficacy of combinatorial personalized cancer therapies targeting both cell-autonomous, as well as non-cell-autonomous pro-tumoral mechanisms. The results disclosed herein therefore highlight the importance of a systematic assessment of the TME composition of cancer patients. Importantly, the observed direct relationship between the immune landscape and the genetic make-up of cancers can greatly facilitate patient stratification for more effective clinical trials. This systematic profiling now needs to be expanded to additional mouse models that include other genetic aberrations frequently observed in prostate cancer, such as amplification of the oncogenes Myc and Ar. Furthermore, the association between different genetic make-ups and cancer-immune phenotypes needs to be thoroughly investigated and integrated in the context of exploratory cancer treatments in preclinical settings. In a recent publication, Patnaik et al. show the efficacy of the tyrosine kinase inhibitor Cabozantinib in a Pten^{pc-/-}; p53^{pc-/-} mouse model and how a massive post-treatment recruitment of PMN cells is critical for a striking anticancer response (Patnaik, A. et al. Cancer Discov CD-16-0778 (2017)). Conversely, in a mouse model combining genetic loss of Pten, p53 and Smad4, Cabozantinib treatment reduces the number of intra-tumoral PMN cells and this, in turn, greatly enhanced the anti-tumor efficacy of immune checkpoint blockade (Lu, X. et al. Nature 543, 728-732 (2017)).

Collectively, the results of the present disclosure strongly suggest that the genetics of cancer play a direct and critical role in shaping the cancer immune-phenotype and that the outcome of combinatorial immunotherapy will be therefore impacted by the tumor genotype, strongly supporting the need of integrated genotypic-immune phenotypic analyses. Importantly, our findings lend support to a cell-autonomous-non-autonomous mode for tumorigenesis dictated by the genetic diversity of cancer and the differential response of the TME that the various genetic make-ups impart (FIG. 6H, FIG. 15B).

In conclusion, the data obtained from genetically engineered mouse models (GEMMs) correlate with what is observed in human clinical cancer samples highlighting the relevance of this co-clinical approach.

The results described herein were obtained using the following materials and methods.

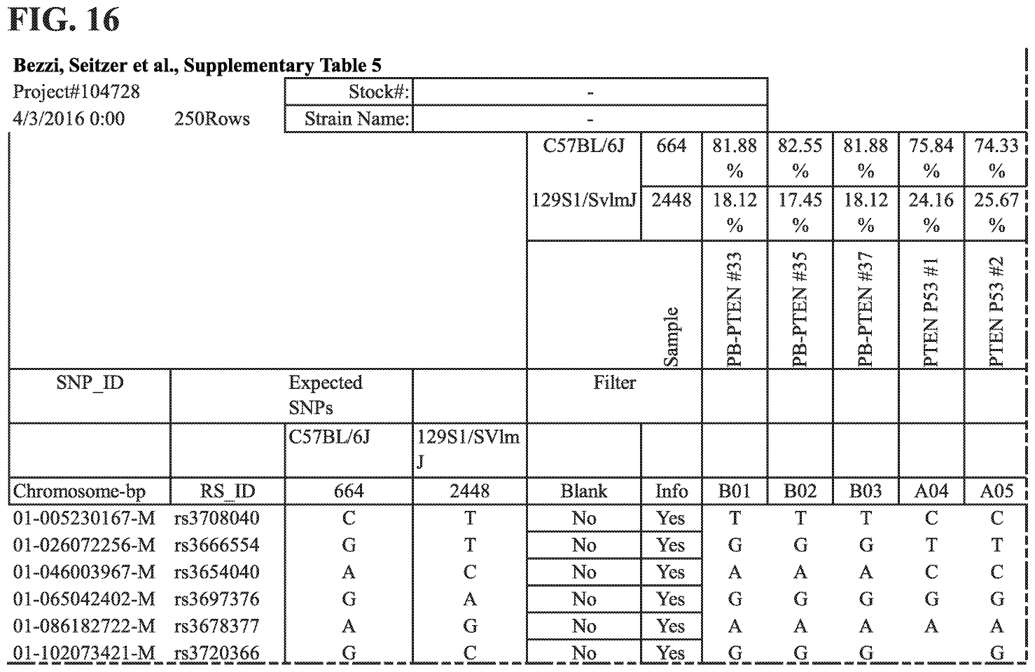

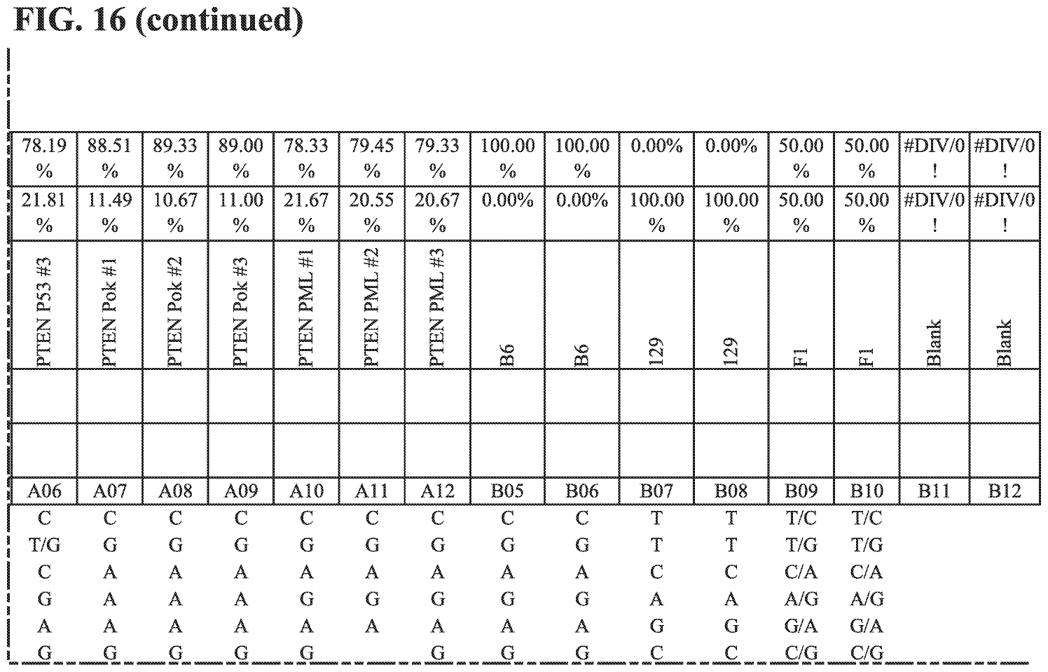

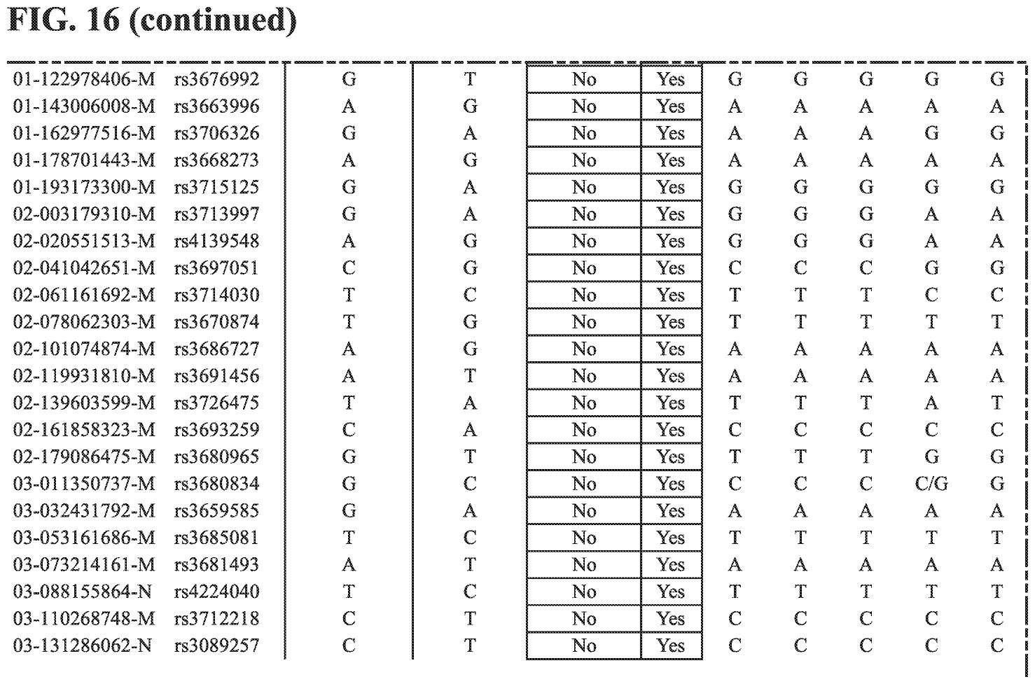

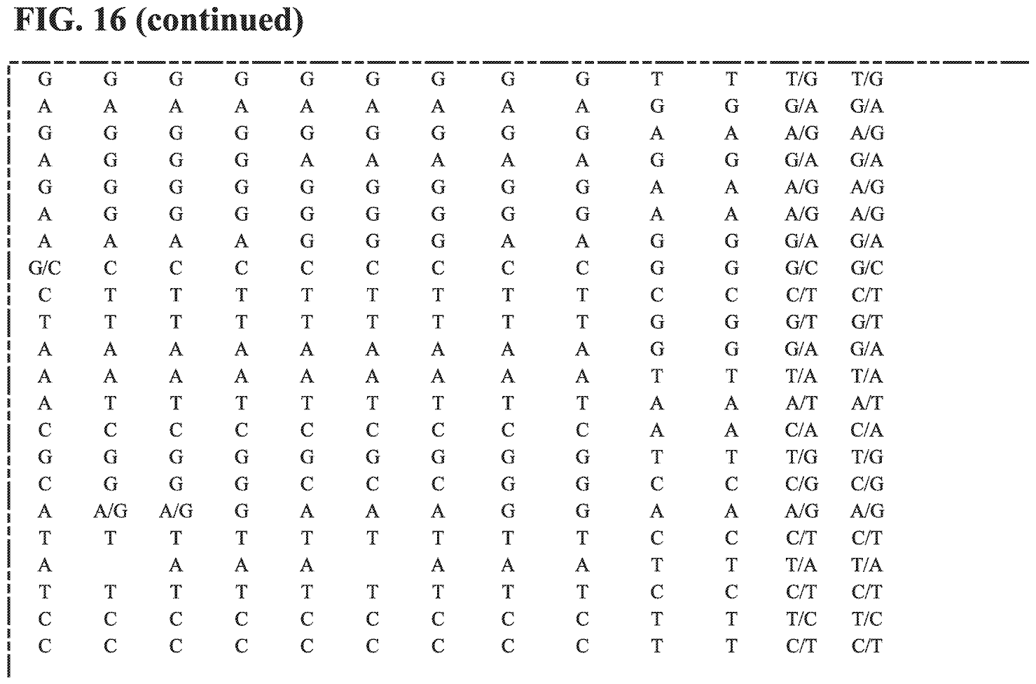

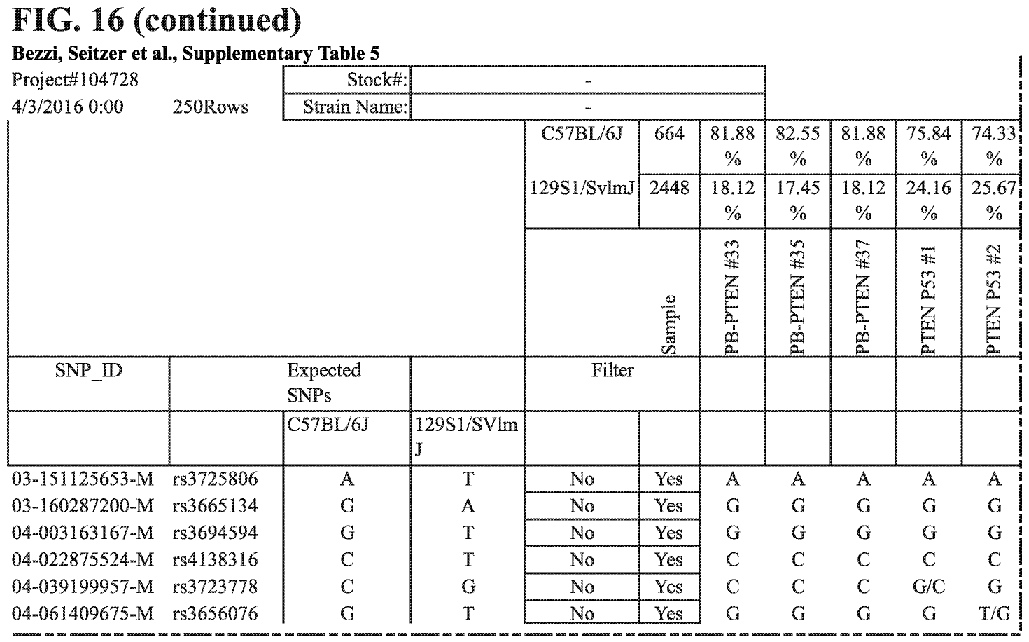

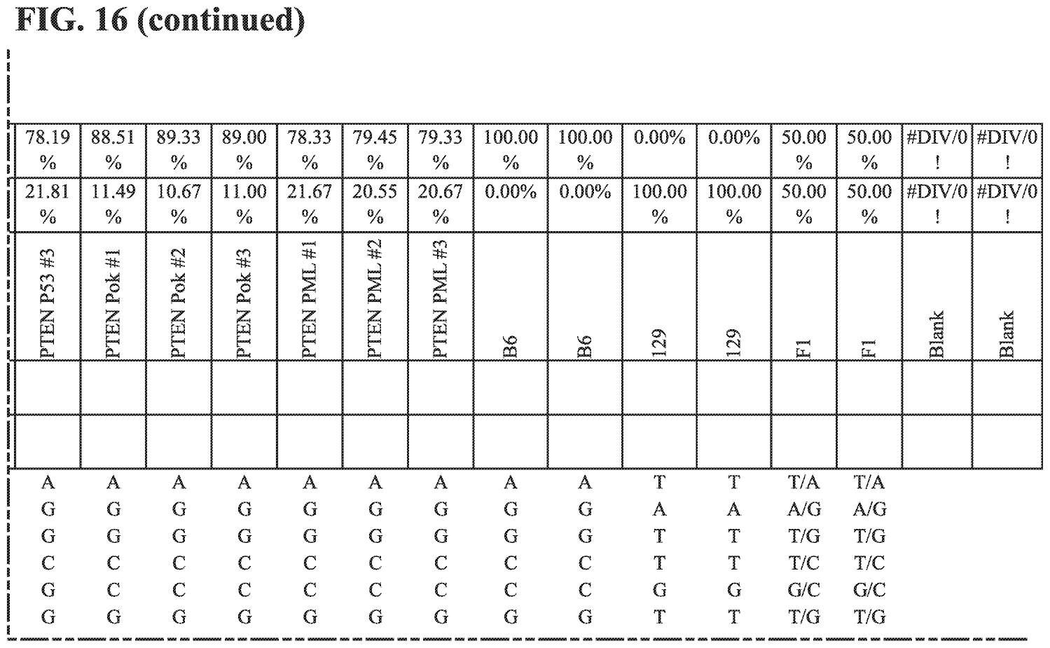

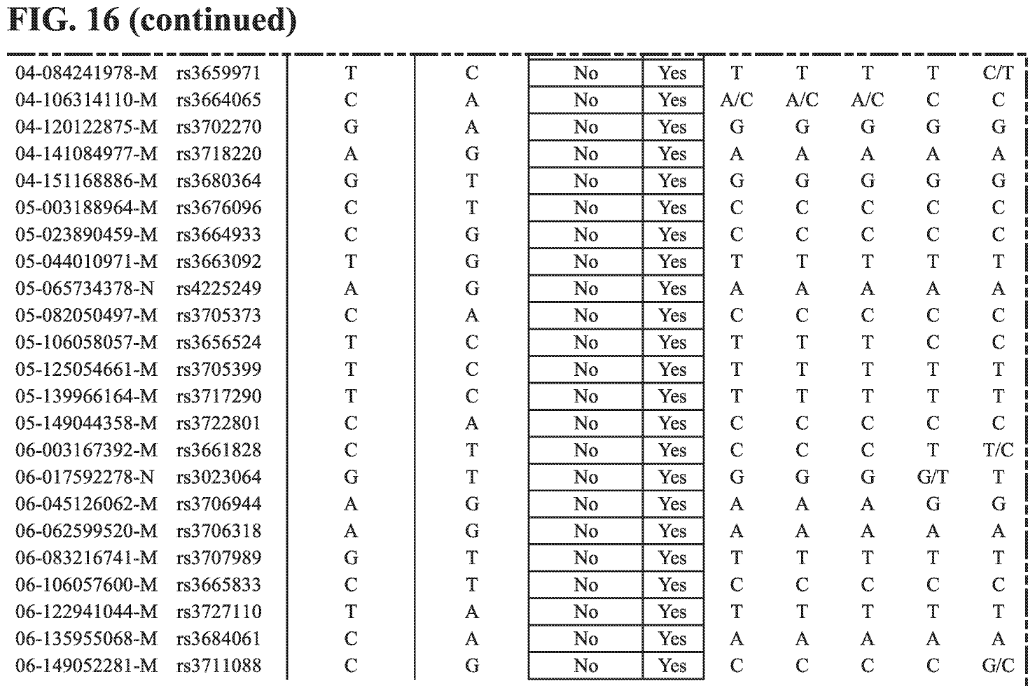

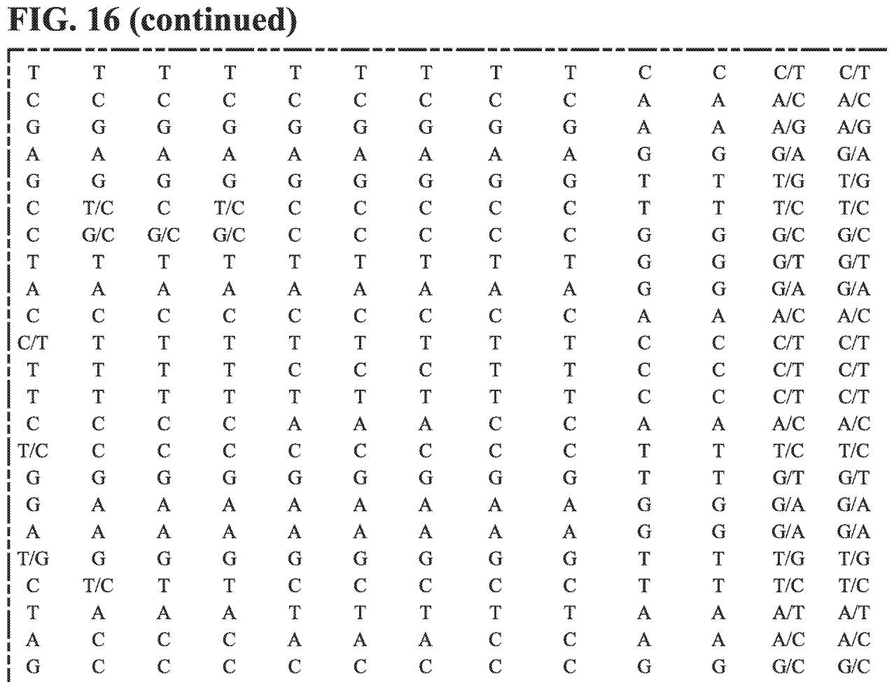

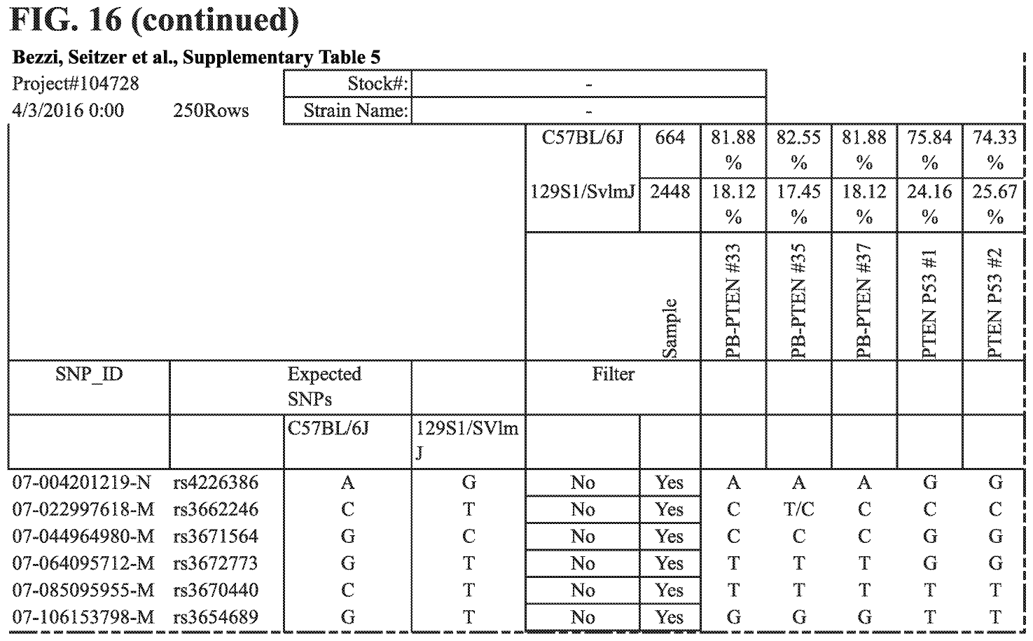

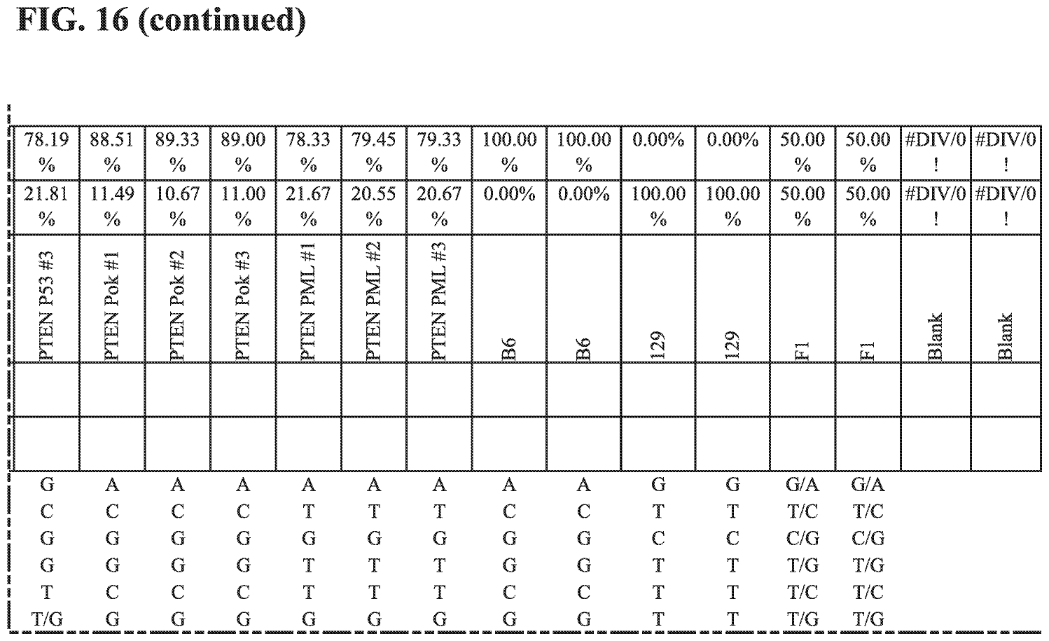

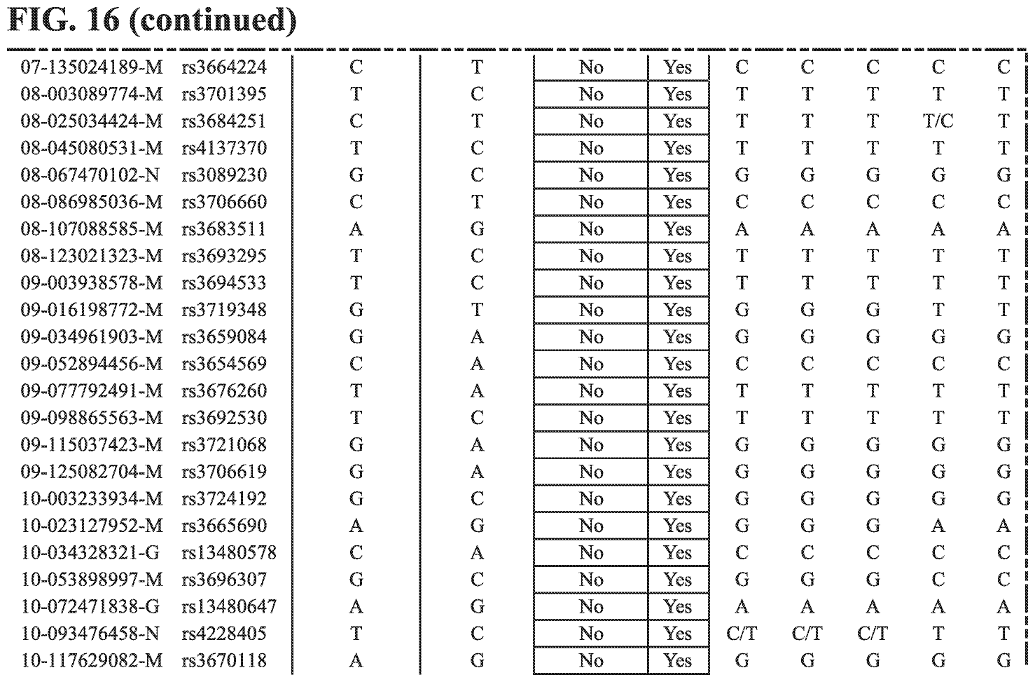

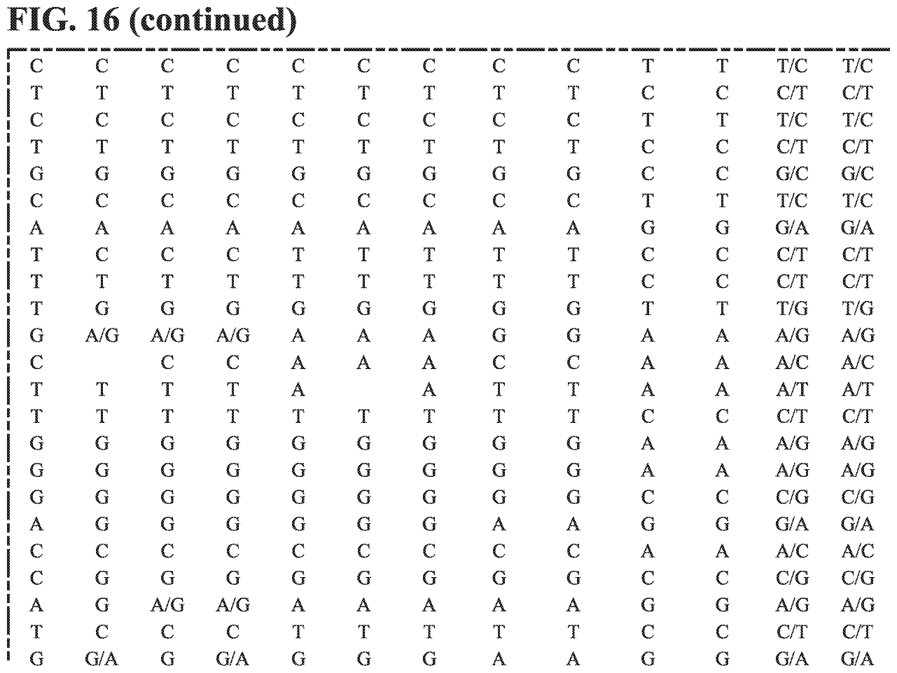

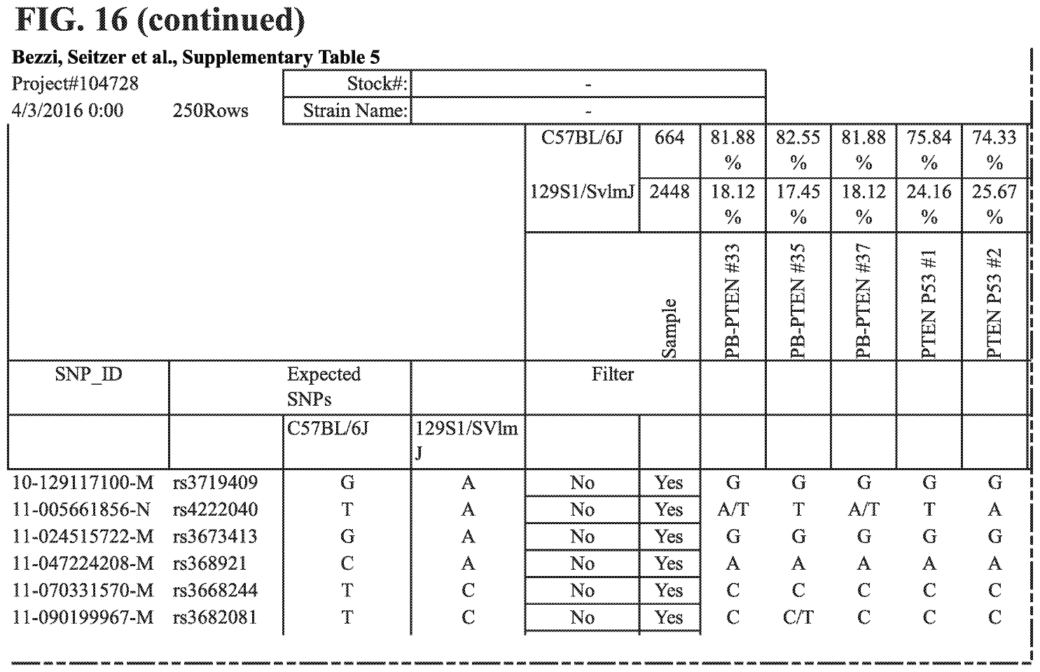

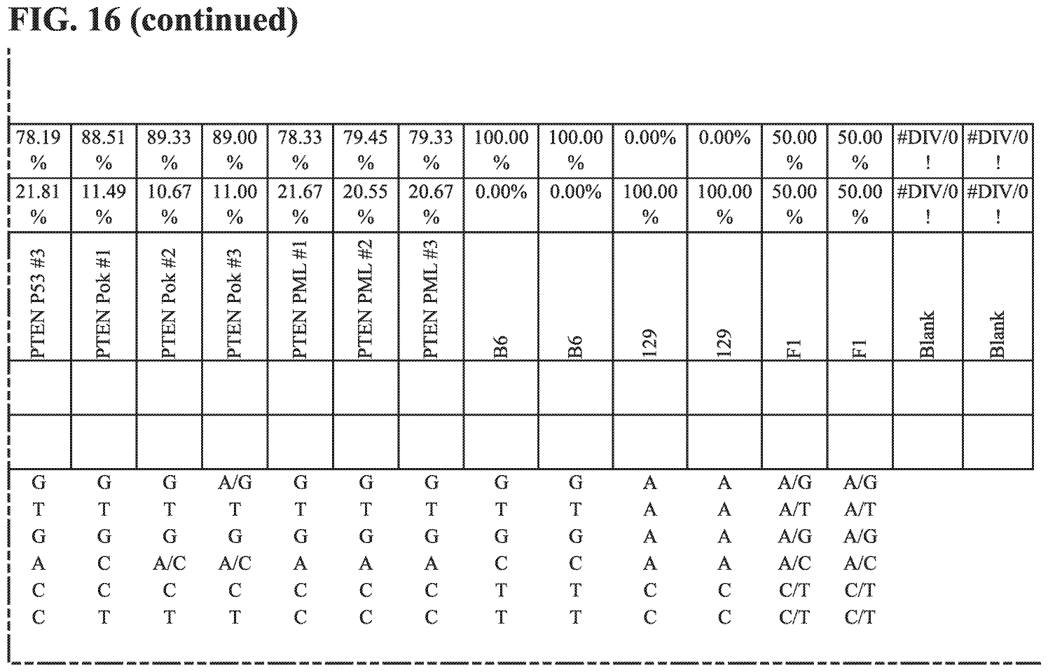

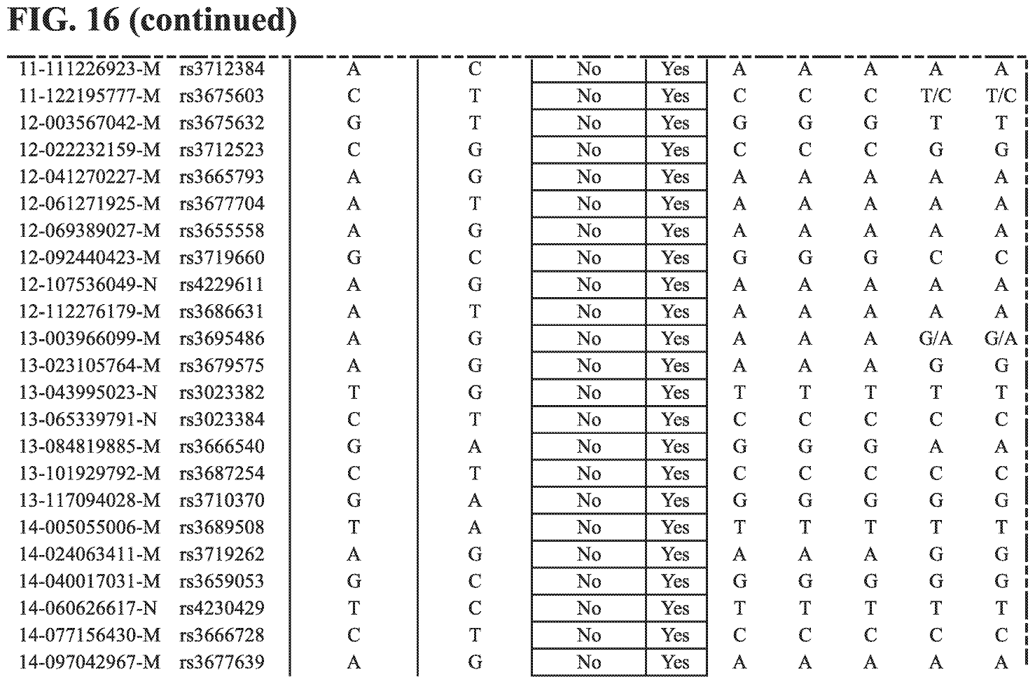

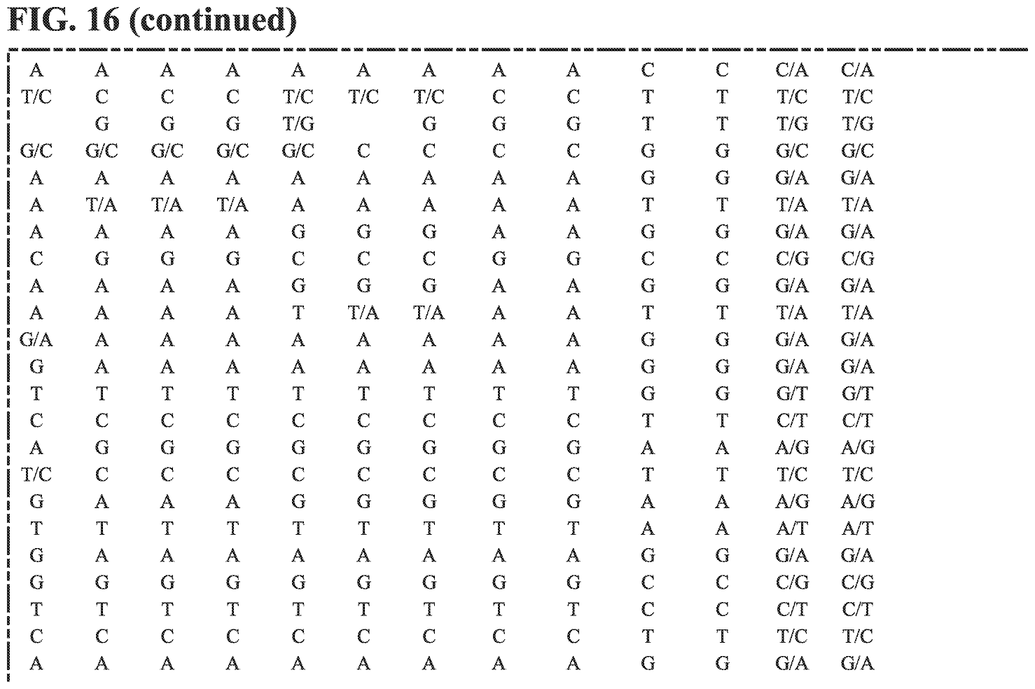

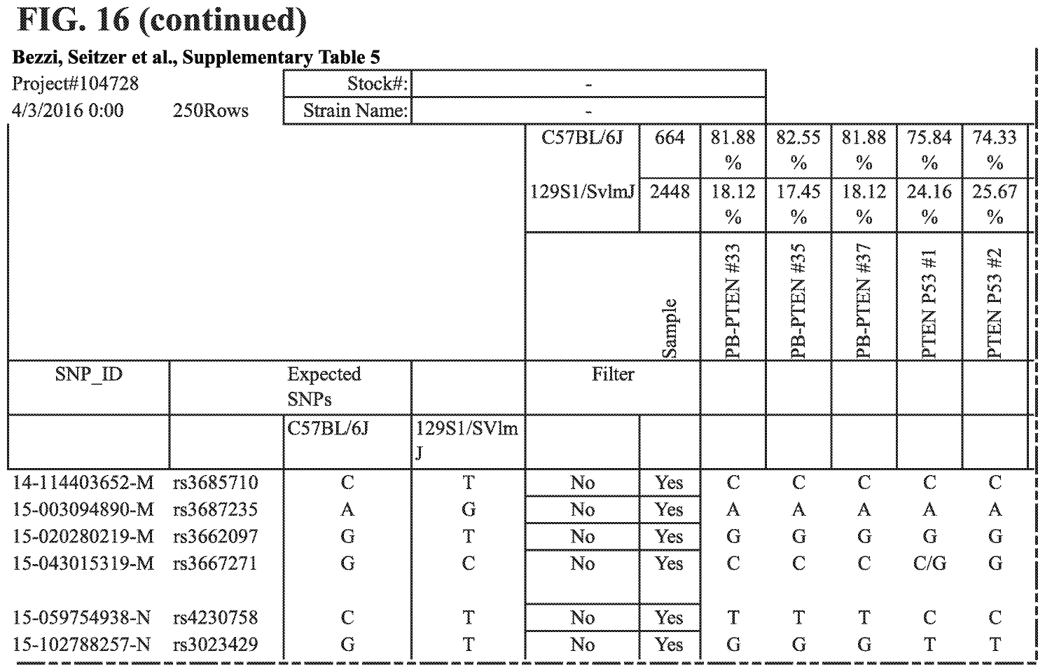

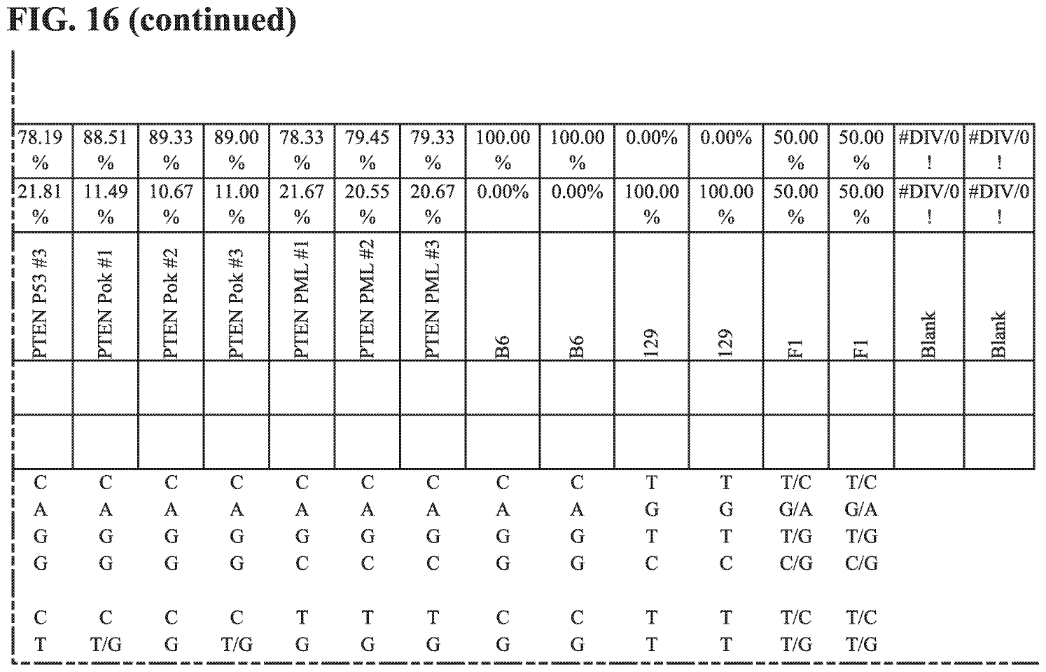

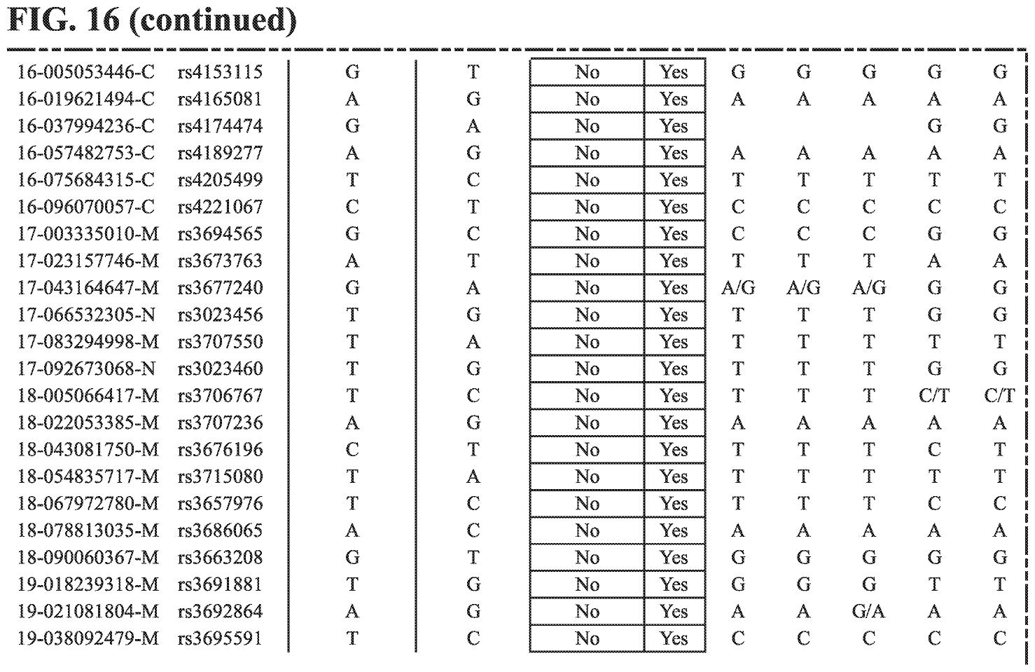

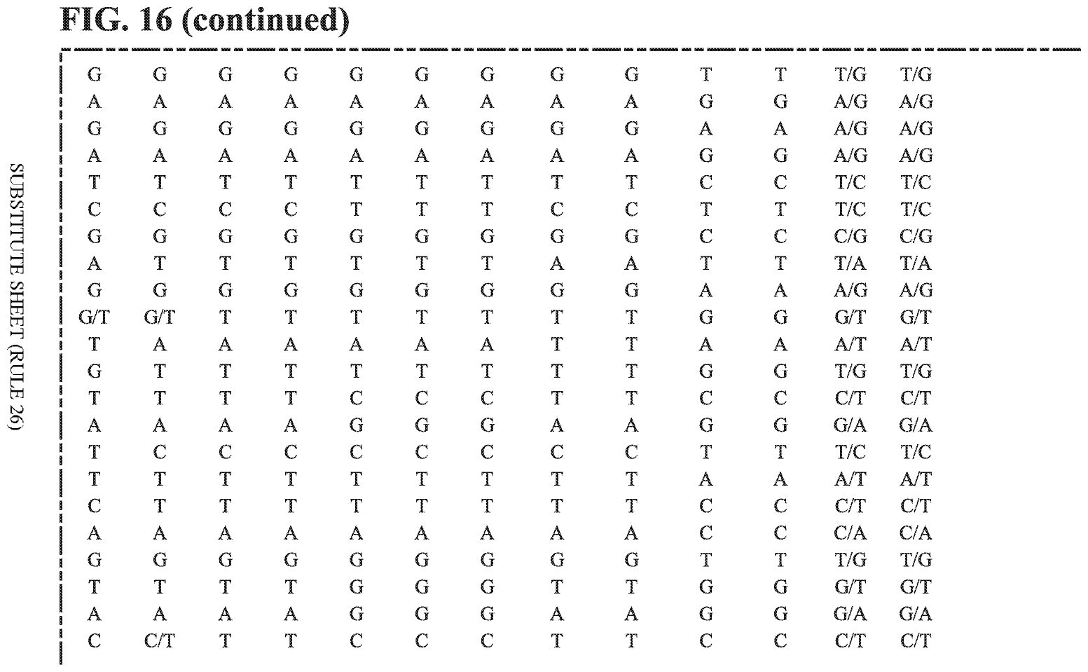

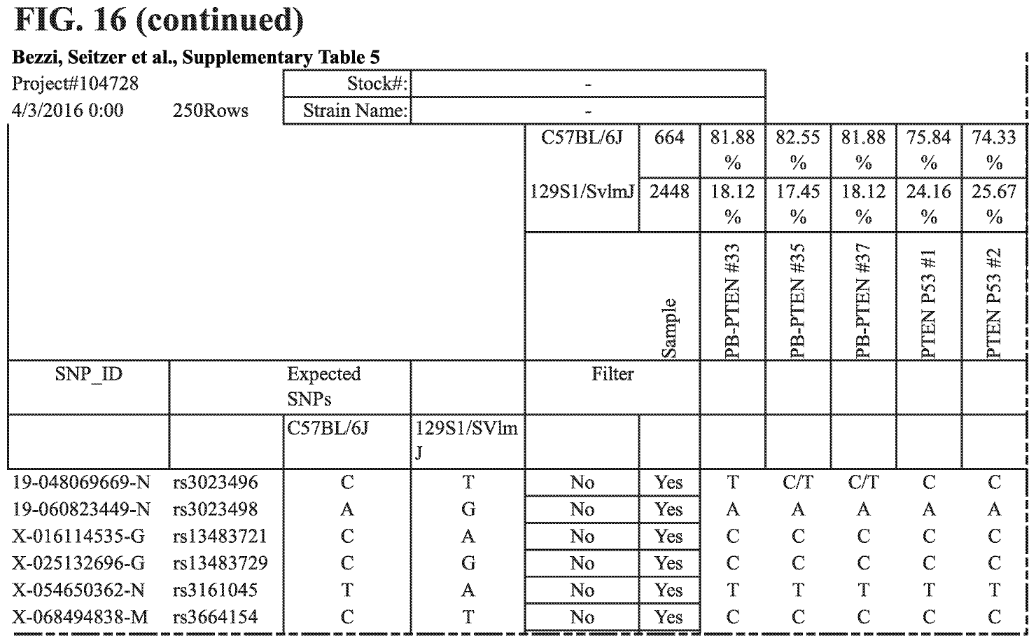

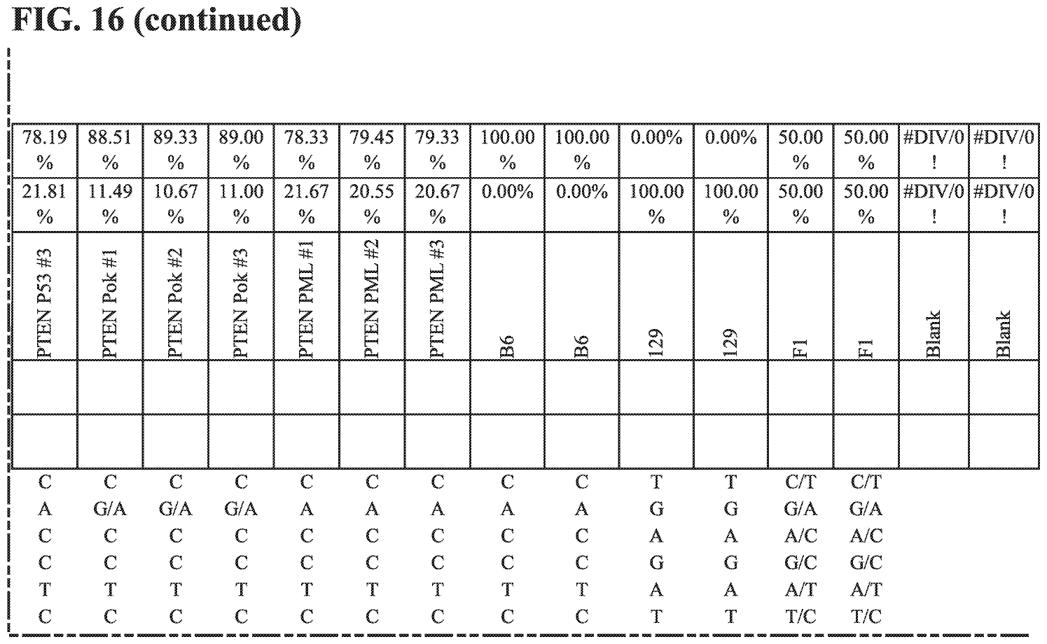

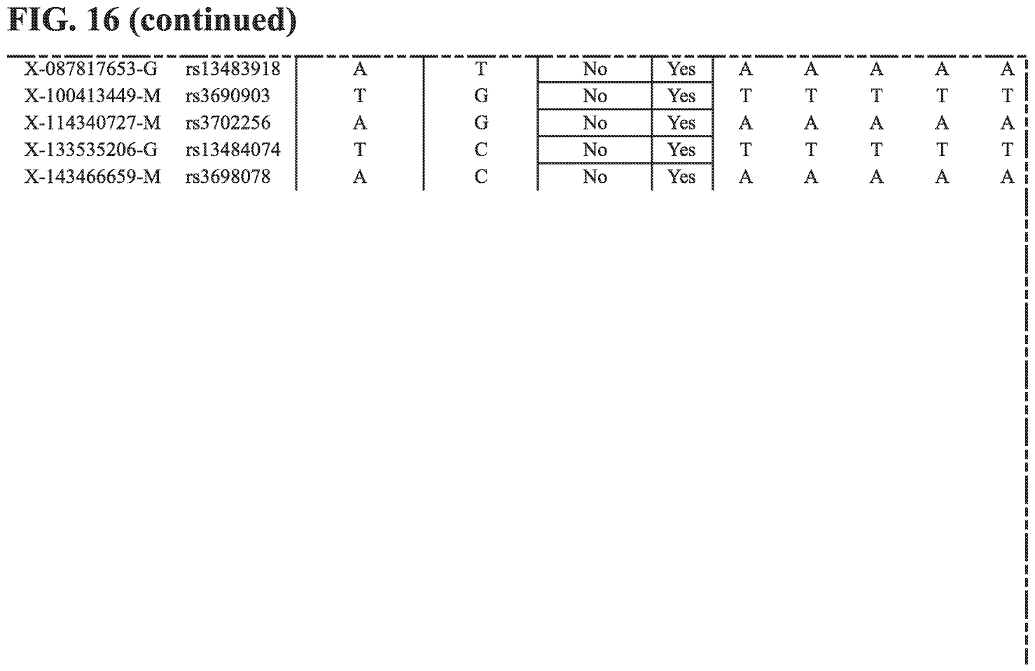

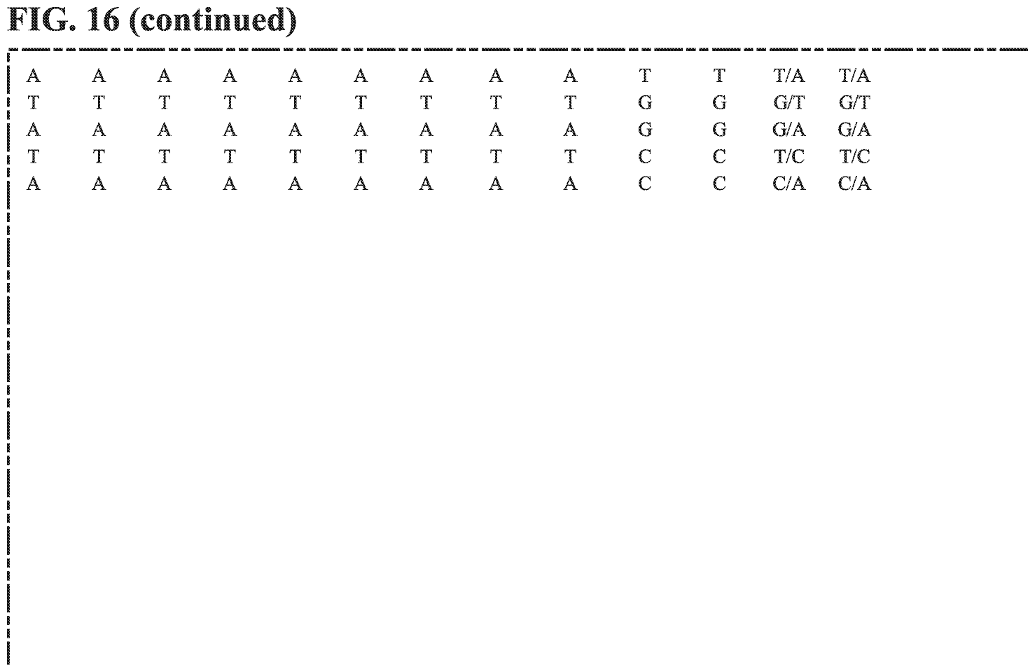

Mice Control, Pten^{pc-/-}, Pten^{pc-/-}; Zbtb7a^{pc-/-}, Pten^{pc-/-}; Trp53^{pc-/-} and Pten^{pc-/-}; Pml^{pc-/-} mice were utilized as described by Trotman, L. C. et al. (PLoS Biol. 2003. 1, e59), Wang, G. et al. (Nat. Genet. 2013. 45, 739-46), Chen, Z. et al. (Nature. 2005. 436, 725-30) and Chen & Pandolfi (manuscript under revision). The mice were maintained in the animal facilities of Beth Israel Deaconess Medical Center (BIDMC)/Harvard Medical School in accordance with institutional rules and ethical guidelines for experimental animal care. All animal experiments were approved by the BIDMC IACUC protocol 066-2011 and 082-2014. The genetic background of the mice is described in FIG. 16.

### In Vivo Drug and Antibody Treatments and MRI Measurement

Mice were allocated at random to experimental groups and studies were performed in an unblended manner. For treatment with the CXCR2 antagonist, SB225002 (Cayman Chemical #13336) was dissolved in DMSO (10 mg/ml) and diluted in vehicle (0.9% NaCl, 0.3% Tween 80) for in vivo administration. Mice (4 months of age) were treated daily for 10 days by intraperitoneal injection (5 mg/kg) and prostate tumor tissue (anterior lobes) was subjected to Flow Cytometry and histological analysis. For MRI analysis, mice (4 months of age) were treated daily for 21 days (Pten^{pc-/-}; Zbtb7a^{pc-/-}), for 14 days (Pten^{pc-/-}; Trp53^{pc-/-}) or for 14 days (Pten^{pc-/-}; Pml^{pc-/-}) by intraperitoneal injection (5 mg/kg). For depletion of Gr-1+/CD11b+ cells, Ly6G-depletion antibody (1A8, BioXcell) and control Rat IgG2a antibody (BioXcell) were diluted in phosphate-buffered saline (PBS) for in vivo administration. Mice (4 months of age) were treated every other day for 10 days by intraperitoneal injection (200-300 μg/mouse). InVivoMAb anti-mouse Ly6G/Ly6C (Gr-1) antibody, clone RB6-8C5 (BE0075, BioXcell), and control Rat IgG2b antibody (BE0090, BioXcell) were diluted in PBS and Pten^{pc-/-}; Trp53^{pc-/-} mice were treated every other day for 14 days by intraperitoneal injection (200 μg/mouse). For neutralization of CXCL5, anti-Mouse CXCL5 antibody (Leinco Technologies) and control Rat IgG2a antibody (BioXcell) were diluted in PBS and injected every other day for 21 days by intraperitoneal injection (20 μg/mouse). Tumor volume quantification was performed by using VivoQuant and Image J software. All mouse prostate MRI imaging analysis was performed at Small Animal Imaging Core at BIDMC and acquired on an ASPECT Model M2 1T tabletop scanner.

### Western Blot Analysis and Immunohistochemistry

For western blotting, cell lysates were prepared by homogenizing tumor tissue with NP40 Buffer (Boston Bioproducts) supplemented with protease (Roche) and HALT phosphatase inhibitor cocktails (Thermo Scientific) and subsequently subjected to SDS-Gel separation (Invitrogen) and western blotting. The following antibodies were used for western blotting: O-Actin (AC-74; Sigma), CXCL5 (R & D Systems #AF433), pIRAK4 (Cell Signaling Technology #11927S), IRAK4 (Cell Signaling Technology #4363P), IκBα (Cell Signaling Technology #4812S), Zbtb7a (hamster anti-Zbtb7a clone 13E9), PTEN (Cell Signaling Technology #9559S), p53 (Cell Signaling Technology #2524S), p21 (Santa Cruz Biotechnology sc-6246) and HSP90 (BD Biosciences BDB610419). Western blots were quantified using Image J software. For immunohistochemistry, tissues and organoids were fixed in 4% paraformaldehyde and embedded in paraffin in accordance with standard procedures. Embedding and hematoxylin and eosin staining of sections were performed by the Histology Core at BIDMC and analyzed by a pathologist. Sections were stained with anti-LY6G (BioLegend #127603), anti-CD45R (Abcam #ab64100), anti-CD3 (Abcam #ab5690), anti-beta Catenin (Abcam #32572), anti-phospho ERK (Cell Signaling Technology #4376), anti-phospho AKT Ser473 (Cell Signaling Technology #4060) and anti-Ki67 (Thermo Scientific #RM-9106) according to manufacturer's recommendations.

Cell Lines and siRNA Transfection

RWPE1 immortalized prostate epithelial cells were obtained from ATCC and tested for mycoplasma with the MycoAlert Mycoplasma Detection Kit (Lonza). RWPE1 cells were maintained in Keratinocyte Serum Free Medium supplemented with bovine pituitary extract (0.05 mg/ml) and human recombinant epidermal growth factor (5 ng/ml). SiRNA targeting Zbtb7a, Sox9 and p53 (SIGMA; final 20 nmol/L) and non-target siRNA control (Thermo Fisher Scientific; final 20 nmol/L) were transfected into RWPE1 cells using Lipofectamine RNAiMAX (Invitrogen). After 48 hours, cells were subjected to mRNA expression analysis. Transient overexpression of Zbtb7a was done as previously described by Wang, G. et al. (Nat. Genet. 2013. 45, 739-46).

### Chromatin Immunoprecipitation

Chromatin Immunoprecipitation (ChIP) was done using the Enzymatic Chromatin Immunoprecipitation Kit (Cell Signaling Technology #9003) following manufacturer's recommendation. For Immunoprecipitation, Zbtb7a antibody (Bethyl Laboratories #A300-549A), Sox9 antibody (Millipore #AB5535), p53 antibody (Cell Signaling Technology #2524), mouse control IgG (Santa Cruz Biotechnology #sc-2025) and rabbit control IgG (Santa Cruz Biotechnology #sc-2027) were used. Analysis of immunoprecipitated DNA was done on the Step One Plus Real Time PCR System from Applied Biosystem using SYBR Green method. Fold Enrichment of ChIP experiments are shown. Primers for the detection of Mia and H19 loci are described previously by Wang, G. et al. (Nat. Genet. 2013. 45, 739-46). Other genes were detected as described in Table 3 shown below.

TABLE 3
Primer Sequences.

| Target | Forward Primer | Reverse Primer |
|---|---|---|
| CXCL5 | ACAACGTCCCTCTC | GGGCAGTGTGGAAA |
| | GGTAGA | GAAGAG |
| CXCL17 | CCAAGTTATCAGTC | CATAACAGGTGAGG |
| | ACCTTC | TGACGCTG |
| p21 | GCTCCCTCATGGGC | TGGCTGGTCTACCT |
| | AAACTCACT | GGCTCCTCT |

### Organoid Culture.

For the generation of mouse prostate cancer organoids, prostate cells were isolated and cultured as described by Drost and Karthaus et al. (Karthaus, W. R. et al. Cell 159, 163-175 (2014); Drost, J. et al. Nat Protoc 11, 347-358 (2016)). Briefly, the prostates of 3 month old mice were dissected and digested in a collagenase type II solution. Single cells were resuspended in Matrigel and cultured as drops in complete prostate organoid medium (advanced DMEM/F12, GlutaMAX, penicillin-streptomycin, (DiHydro) testosterone, B27, N-acetylcystein, EGF, R-Spondin, Noggin, A83-01, Y27632).

### Gr1+ Cells and Monocytes Isolation, Culture and Migration Assay.

Gr1+ cells and monocytes were isolated from the bone marrow (tibias and femurs) of C57BL/6 wild type, 3 months old mice using the MACS Myltenyi Biotec Cell Isolation system according to the manufacturer's instruction. For monocytes isolation the Monocyte Isolation Kit (BM) (Miltenyi 130-100-629) was used, whereas Gr1 positive cells were isolated using the antibody Anti-Gr-1-Biotin, clone RB6-8C5 (Miltenyi 130-101-894). Red blood cells were lysed with the ACK lysis buffer (ThermoFisher Scientific A1049201). Total bone marrow cells and Gr1+ cells were cultured for 4 days. Briefly, 40 ng/ml GM-CSF (PeproTech #315-03) and 40 ng/ml IL-6 (PeproTech #216-16) were added to the control medium RPMI 1640 (ThermoFisher Scientific 11875-093), supplemented with penicillin-streptomycin, 10% FBS, 10 mM HEPES, 20 μM 2-Mercaptoethanol. Either recombinant mouse CXCL5 (BioLegend #573302) or recombinant mouse CXCL17 (BioLegend #585402) was added at the beginning of the experiment (200 nM). For the migration assay 2.5×10⁵ MACS sorted cells were resuspended in 100 μL of either RPMI 1640 control medium or organoid complete medium and placed on the upper well of the transwell system (5 μm, Corning #160241). The migration assay with recombinant proteins was performed by adding to the bottom well 600 μL of RMPI1640 control medium supplemented with the indicated amount of either CXCL5 or CXCL17. The migration assay with organoid conditioned medium was performed by adding to the bottom well 600 μL of medium collected over 5 days of culture of prostate organoids with the indicated genotype. The migration of cells was quantified by flow cytometry, 15 seconds acquisition time, using BD LSR II flow cytometer.

### Cytospin

To perform Cytospins, 2×10⁵ sorted granulocytes were resuspended in PBS containing 2% fetal bovine serum (FBS) (2% FBS/PBS) and spun onto slides with 250 rpm for 3 min in a slide centrifuge. Slides were subsequently fixed in methanol and stained with May Grunwald/GIEMSA.

### Flow Cytometry

For Flow Cytometry, spleen and lymph node single cell suspensions were prepared by mashing the tissue in 2% FBS/PBS. Tumor and control prostate tissue (from anterior lobes) single cell suspension was prepared by mincing the tumor and digestion with Collagenase Type I (Life Technologies #17018029) in 10% DMEM (GIBCO) for 1 hr at 37° C. Cell suspensions were passed through 100 μM cell strainers to obtain single cell suspensions. Blood samples and single cell suspensions were re-suspended in 1-2 ml of ACK red cell lysis buffer (GIBCO) and lysed on ice for 1 minute. Cell suspensions were then washed in 2% FBS/PBS, centrifuged and re-suspended in 0.5-1 ml of 2% FBS/PBS. For flow cytometry, 100 μl of cell suspension was stained in a 96-well U-bottom plate and the following antibodies were used: CD45.2-Pacific Blue (BioLegend #109820), CD45.2-APC (eBioscience #17-0454-82), CD45.2-FITC (eBioscience #11-0454-85), Gr-1-FITC (eBioscience #11-5931), Gr-1-PE (eBioscience #12-5931-81), CD11b-PECy7 (eBioscience #25-0112), CD11b-FITC (eBioscience #11-0112-82), Ly6C-PE (eBioscience #12-5932-80), Ly6G-APC-Cy7 (BioLegend #127623), Ly6G-APC (BioLegend #127613), CD3-PE (eBioscience #12-0031), CD4-APC (BioLegend #100515), CD4-PE-Cy7 (eBioscience #25-0042-82), CD8-APC (BioLegend #100721), CD8-FITC (eBioscience #11-0081-85), B220-FITC (eBioscience #11-0452), CD19-PerCP-Cy5.5 (eBioscience #45-0193), F4/80-APC (eBioscience #17-4801), CD44-FITC (BD Pharmingen #561859), CD62L-APC (BioLegend #104411), CD206-PE (BioLegend #141705) and CD49f-APC (eBioscience #17-0495). All antibodies were used 1:100. To assess cell viability, cells were incubated with either DAPI or TO-PRO3 prior to FACS analysis. Foxp3 staining was done using FOXP3 Fix/Perm Buffer Set (BioLegend) and cells were stained by Foxp3-FITC antibody (eBioscience #11-5773). All staining mixtures were analyzed on a BD LSR II flow cytometer (Becton Dickinson). Resulting profiles were further processed and analyzed using the FlowJo 8.7 software.

### Cell Sorting

For cell sorting of the Gr-1+/CD11b+ cell population, CD45-/CD49f+ cell population and the CD4+ cell population, tumor tissue, blood and spleen was prepared as described above. After red blood cell lysis in 1-2 ml of ACK lysis buffer, cells were immunostained with anti-CD45-Pacific Blue, anti-Gr-1-FITC, anti-CD11b-PECy7, anti-CD49f-APC and CD4-APC, washed and sorted on a BD™ FACSAria IIu SORP cell sorter (Becton Dickinson). For cell sorting of the Ly6C+/Ly6G− and Ly6C+/Ly6G+ cell populations, cells were immunostained with anti-CD11b-FITC, anti-Ly6C-PE and anti-Ly6g-APC.

### In Vitro Treg Cells Induction Assay

CD4+ T cells were sorted from spleen of tumor free control mice as described above. Purified CD4+ T cells were co-cultured with Gr1+/CD11b+ cells from Pten^{pc-/-}; Zbtb7a^{pc-/-} and Pten^{pc-/-}; Trp53^{pc-/-} tumors at 3 month of age at a ratio of 4:1 (T cells/Gr-1+/CD11b+ cells) in the presence of recombinant murine interleukin 2 (10 ng/ml, R&D Systems). After 4 days culture, cells were harvested and subjected to flow cytometry analysis as described above.

### RT-PCR and Microarray Analysis

Microarray analysis and gene set enrichment analysis on mouse tumor tissue were conducted and analyzed as previously described by Palucka, A. K. et al. (Palucka, A. K. et al. Cell 164, 1233-1247 (2016)). For mRNA expression levels, tissue from indicated mice were homogenized in TRIZOL (Life Technologies #15596026) and RNA was extracted WO 2018/044937 PCT/US2017/0491%6 according to manufacturer's recommendation. RNA was further purified with the Pure Link RNA Mini Kit (Life Technologies #12183025) following the manufacturer's recommendation. For mRNA expression analysis of human cell lines or separated Gr-1 positive cells, RNA was isolated using Pure Link RNA Mini Kit following manufacturer's recommendations. RNA was reverse transcribed into cDNA by the High Capacity cDNA Reverse Transcription Kit (Life Technologies #4368814). Expression levels were measured via relative quantification on the Step One Plus Real Time PCR System from Applied Biosystem using SYBR Green method. Data are shown as fold change or expression values as indicated. Primer sequences are included in Table 4 and Table 5 below.

TABLE 4
Primer sequences targeting
mouse genes used for qRT-PCR

| Gene | Forward | Reverse |
|---|---|---|
| Actin | CGTCGACAACGGC | TGGGCCTCGTCAC |
| | TCCGGCA | CCACATAGG |
| CCL1 | CAGGATGTTGACA | CATCTTTCTGTAA |
| | GCAAGAG | CACTGG |
| CCL2 | GGCCTGCTGTTCA | CTGCTGGTGATCC |
| | CAGTTG | TCTTGTAG |
| CCL3 | CTGCAACCAAGTC | GCCGGTTTCTCTT |
| | TTCTCAG | AGTCAGG |
| CCL4 | CTTCTGTGCTCCA | CTGTCTGCCTCTT |
| | GGGTTCTC | TTGGTCAG |
| CCL5 | GCTGCTTTGCCTA | TCGAGTGACAAAC |
| | CCTCTCC | ACGACTGC |
| CCL7 | GCTTTCAGCATCC | GACTACTGGTGAT |
| | AAGTGTG | CCTTCTG |
| CCL20 | GCCTCTCGTACAT | CCAGTTCTGCTTT |
| | ACAGACGC | GGATCAGC |
| CCL28 | GTGTGTGGCTTTT | TGCATGAACTCAC |
| | CAAACCTCA | TCTTTCCAG |
| CXCL1 | ACTGCACCCAAAC | TGGGGACACCTTT |
| | CGAAGTC | TAGCATCTT |
| CXCL2 | CCAACCACCAGGC | GCGTCACACTCAA |
| | TACAGG | GCTCTG |
| CXCL3 | GATTTTGAGACCA | CTCTTCAGTATCT |
| | TCCAGAGC | TCTTGATG |
| CXCL5 | TGCATTCCGCTTA | CAGAAGGAGGTCT |
| | GCTTTCT | GTCTGGA |
| CXCL7 | CACTTCATAACCT | CACAGTGAACTCC |
| | CCAGATC | TGGCCTGTAC |
| CXCL9 | GGAGTTCGAGGAA | GGGATTTGTAGTG |
| | CCCTAGTG | GATCGTGC |
| CXCL10 | CCAAGTGCTGCCG | GGCTCGCAGGGAT |
| | TCATTTTC | GATTTCAA |
| CXCL12 | GTAAACCAGTCAG | GCTTTCTCCAGGT |
| | CCTGAG | ACTCTTG |
| CXCL14 | GGAAATGAAGCCA | GATGAAGCGTTTG |
| | AAGTACC | GTGCTCTG |
| CXCL15 | CAAGGCTGGTCCA | TGCTATCACTTCC |
| | TGCTCC | TTTCTGTTGC |
| CXCL16 | GGACTGCTTTGAG | CTGAGTGCTCTGA |
| | CGCAAAG | CTATGTG |
| CXCL17 | AGGTGGCTCTTGG | GGTGACATCGTTT |
| | AAGGTG | GAGAAATTGC |
| IL1beta | GAAATGCCACCTT | TGGATGCTCTCAT |
| | TTGACAGTG | CAGGACAG |
| S100A9 | GCACAGTTGGCAA | TGATTGTCCTGGT |
| | CCTTTATG | TTGTGTCC |
| S100A8 | AAATCACCATGCC | CCCACTTTTATCA |
| | CTCTACAAG | CCATCGCAA |
| Arginase | TTTTTCCAGCAGA | AGAGATTATCGGA |
| | CCAGCTT | GCGCCTT |
| iNOS | TTCTGTGCTGTCC | TGAAGAAAACCCC |
| | CAGTGAG | TTGTGCT |
| IL10 | ATCGATTTCTCCC | TGTCAAATTCATT |
| | CTGTGAA | CATGGCCT |
| CD40 | GTCGGCTTCTTCT | CATCACGACAGGA |
| | CCAATCAG | ATGACCAG |

TABLE 5
Primer sequences targeting
mouse genes used for qRT-PCR

| Gene | Forward | Reverse |
|---|---|---|
| Actin | TGGCACCCAGCACA | CTAAGTCATAGTCC |
| | ATGAA | GCCTAGA |
| CXCL5 | CTGTTGGTGCTGCT | CGAACACTTGCAGA |
| | GCTGCTG | TTACTG |
| CXCL17 | TGCTGCCACTAATG | CTCAGGAACCAATC |
| | CTGATGT | TTTGCACT |
| p21 | GACCTGTCACTGTC | CTTCCTCTTGGAGA |
| | TTGTAC | AGATCAG |

### Gene Expression Profiling

The Cancer Genome Atlas Prostate Adenocarcinoma (TCGA-PRAD) data and Robinson metastatic prostate cancer data were downloaded from the cbioportal web site (http://www.cbioportal.org/). See Cerami, E. et al. (Cancer Discov. 2012. 2, 401-4), Gao, J. et al. (Sci. Signal. 2013. 6, 11). Normalized gene expression data were logarithm-transformed using base 2. Box plot and hierarchical clustering analyses were conducted with R programming. Samples with ZBTB7A expression below quantile 0.205 were counted low, above quantile 0.795 counted as high. Samples with homozygous deletion of PTEN, TP53, or PML were counted as altered, “alt”. Standard t tests or Wilcoxon signed-rank tests were conducted to calculate the significance of number of samples falling into different categories in the boxplot. Z score test for two population proportions was conducted for FIG. 6F between the ratios, e.g. PMN-high ratio in PML-alt group versus that ratio in “Pten-alt, Zbtb7a-low” group. The list of genes used to generate the gene signatures used the bioinformatics analysis are in Table 2. The PMN signature has been generated by slightly modifying the MDSC gene signature used by Wang et al. (Wang, G. et al. Cancer Discov. 6, 80-95 (2016)). The Mo-MDSC signature has been generated including Mo-MDSC and M2-like TAM human genes highlighted in the FIG. 1 of the review recently published by Bronte and colleagues (Ugel, S., et al. Journal of Clinical Investigation 125, 3365-3376 (2015)). The T cell-signature is the one used by Spranger et al. (Spranger, S. et al. Nature 523, 231-235 (2015)).

### Statistical Analysis

No statistical method was used to predetermine sample size. There were no mice excluded from experiments. For all statistical analyses GraphPad Prism 6 software was used and the analysis was done by a two-tailed unpaired student's t-test. Analysis of specimens with high expression of CXCL5 and CXCL17 was carried out performing a Fisher's exact test. Values of p<0.05 were considered statistically significant. *P<0.05; **P<0.01; ***P<0.001 (t-test).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating a cancer characterized by a deficiency in Pten and p53, the method comprising administering an agent that inhibits the expression or activity of CXCL17 to a subject having a cancer identified as Pten, Zbtb7a/Pokemon, p53, and/or Pml deficient.

2. A method of treating a subject having cancer, the method comprising
(a) obtaining a biological sample from the subject;
(b) detecting a tumor suppressor selected from the group consisting of Pten, Zbtb7a/Pokemon, p53, and Pml in the biological sample, wherein a deficiency in the tumor suppressor indicates the subject could benefit from CXCL17 inhibition; and
(c) administering an agent that inhibits CXCL17 expression or activity to the subject, thereby treating the cancer.

3. The method of claim 1, wherein the cancer is prostate cancer, breast cancer, colorectal cancer, gastric cancer, pancreatic cancer, or any other cancer of epithelial origin.

4. Tag method of claim 1, wherein the method treats prostate cancer.

5-6. (canceled)

7. The method of claim 1, wherein the agent is an inhibitory nucleic acid molecule that inhibits the expression of a CXCL17 protein.

8. The method of claim 3, wherein the inhibitory nucleic acid molecule is an antisense molecule, siRNA or shRNA.

9. The method of claim 1, wherein the agent is CID-2745687 or ML-145.

10. (canceled)

11. The method of claim 1, wherein the cancer is deficient in Pten and p53; deficient in Pten and Zbtb7a/Pokemon; deficient in Pten, Zbtb7a/Pokemon and p53; or deficient in Pten, p53, Zbtb7a/Pokemon, and Pml)

12. A mouse comprising a prostate cancer organoid, wherein the organoid expresses endogenous or recombinant CXCL17.

13. The mouse of claim 11, wherein the mouse fails to express or expresses undetectable levels of one or more tumor suppressors selected from the group consisting of Pten, Zbtb7a/Pokemon, p53, and Pml.

14. The mouse of claim 12, wherein the cell is a prostate epithelium cell.

15. A method for obtaining an immune-competent murine model for drug screening, the method comprising
(a) obtaining one or more neoplastic cells expressing CXCL17 from the mouse of claim 12;
(b) culturing the neoplastic cell in vitro to obtain one or more cancer organoids;
(c) implanting the cancer organoid into a syngeneic mouse not having the defined genetic lesion, thereby obtaining an immune-competent murine model for drug screening.

16. A method of identifying an anti-cancer therapeutic agent for a subject having one or more defined genetic lesions, the method comprising
(a) obtaining one or more neoplastic cells expressing CXCL17 from the mouse of claim 12;
(b) culturing the neoplastic cell in vitro to obtain one or more cancer organoids;
(c) implanting the cancer organoid into an immune competent syngeneic mouse;
(c) administering one or more candidate agents to the syngenic mouse; and
(d) assaying the biological response of the organoid or syngeneic mouse to the candidate agent.

17. The method of claim 15, wherein the defined genetic lesion is in a tumor suppressor gene selected from the group consisting of Pten, Zbtb7a/Pokemon, p53, and Pml.

18. The method of claim 17, wherein the genetic lesion is a missense mutation, nonsense mutation, insertion, deletion, or frameshift.

19. The method of claim 17, wherein the defined genetic lesion results in a loss of expression or function in the tumor suppressor.

20. The method of claim 16, wherein the candidate agent is a polypeptide, polynucleotide, or small compound.

21. The method of claim 20, wherein the polypeptide is an anti-CXCL17 antibody.

22. The method of claim 16, wherein assaying the biological response comprises detecting tumor vascularization, the profile of tumor infiltrating myeloid-derived suppressor cell, chemotaxis of myeloid-derived suppressor cells, correlations of CXCL17 expression levels with changes in Treg numbers, Th1 versus Th2 cytokine profiles, tumor growth, and/or murine survival.

23. A method of identifying an anti-cancer therapeutic agent for a subject having one or more defined genetic lesions, the method comprising
(a) obtaining one or more neoplastic cells expressing CXCL17 from a set of the mouse of claim 12, each having one or more defined genetic lesions;
(b) culturing the neoplastic cells in vitro to obtain a set of cancer organoids;
(c) implanting each cancer organoid into an immune competent syngeneic mouse;
(c) administering one or more candidate agents to the syngenic mouse; and
(d) assaying the biological response of the organoid or syngeneic mouse to the candidate agent, wherein a reduction in tumor growth or an increase in mouse survival indicates that the candidate agent is useful for the treatment of a subject having a corresponding defined genetic lesion.


SEQUENCE LISTING (79 sequences)

SEQ ID NO: 1 - protein, 119 aa, Homo sapiens
MKVLISSLLLLLPLMLMSMVSSSLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 2 - DNA, 1209 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaacgaacctgatcattatagatattctgacaccactgactctgatccagagaatgaaccttttgatgaagatcagcatacacaaattacaaaagtc

SEQ ID NO: 3 - protein, 403 aa, Homo sapiens [sequence omitted]

SEQ ID NO: 4 - DNA, 6318 bp, Homo sapiens
actgccgcctcccggcccctcggagggagccagcccagccgcagccgccgccaccgccgccgccggggccgggccccctcgccgctgccccgggaaggaggtctcggcgcggaagatggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcagatcctggcggccgacgcgggcgccgacgccgggcagctggaccttgtagatcaaattgatcagcgcaacctcctccgcgccaaggagtacctcgagttcttccagagcaaccccatgaacagcctgccccccgcggccgccgccgccgctgccagcttcccgtggtccgcctttggggcgtccgatgatgacctggatgccaccaaggaggccgtggccgccgctgtggccgccgtggccgcgggcgactgcaacggcttagacttctatgggccgggccccccggccgagcggcccccgacgggggacggggacgagggcgacagcaacccgggtctgtggccagagcgggatgaggacgcccccaccgggggtctctttccgccgccggtggccccgccggccgccacgcagaacggccactacggccgcggcggagaggaggaggccgcctcgctgtcggaggcggcccccgagccgggcgactctccgggcttcctgtcgggagcggccgagggcgaggacggggacgggcccgacgtggacgggctggcggccagcacgctgctgcagcagatgatgtcatcggtgggccgggcgggggccgcggcgggggacagcgacgaggagtcgcgggccgacgacaagggcgtcatggactactacctgaagtacttcagcggcgcccacgacggcgacgtctacccggcctggtcgcagaaggtggagaagaagatccgagccaaggccttccagaagtgccccatctgcgagaaggtcatccagggcgccggcaagctgccgcgacacatccgcacccacacgggcgagaagccctacgagtgcaacatctgcaaggtccgcttcaccaggcaggacaagctgaaggtgcacatgcggaagcacacgggcgagaagccgtacctgtgccagcagtgcggcgccgcctttgcccacaactacgacctgaagaaccacatgcgcgtgcacacgggcctgcgcccctaccagtgcgacagctgctgcaagaccttcgtccgctccgaccacctgcacagacacctcaagaaagacggctgcaacggcgtcccctcgcgccgcggccgcaagccccgcgtccggggcggggcgcccgaccccagcccgggggccaccgcgacccccggcgcccccgcccagcccagctcccccgacgcccggcgcaacggccaggagaagcactttaaggacgaggacgaggacgaggacgtggccagccccgacggcttgggccggttgaatgtagcgggcgccggtggaggaggtgacagcggaggtggccccggggccgccaccgacggtaacttcacagccggactcgcctaaaaaccaaaaagagaaaacagaaacccgagaaagagagagacagagagagagaaaaaaaatcacccaccacccccccaaaaacacaaaaaaagaaaatctatctatatacagatatctatatctatatatatatatacagatatatatatatgacgcgtcacagaatctagggtagcgctttctcagatttccctcctttctgacgtttttctccctccgcaggggccccggccctccctggctccccttccccccaccaccccatcgctgggtttcggggcttggtttggggttttttgtaggacacaaggaatccgagaccccgcacagccccctgggcacccggcatggggcctggggcccgatccgaggccctgggctggggggagggtagacgtgggggcgctggggggggactggggtgggcttttaatttcctcccctcgctggtttctatgagtctttcagacaagaccttaaatgatttctgtctgctctgagcggacgttaaaatgggcccccgtcccccgacccgcaccctccttcctcagggcacttactaagggaggggtctccctctccatctccccagtggcctccccgcctccaaccctgcctgcggcctccccccgtcgcccaccccacgtctcctggccactgagacacaaacctatttatttctaggcctggagaaaggagatcggactggggttcccggtggggcgccaggatggctcctgggggtgctcctgccgccttccttcacggcacttacaaccggcgggacccccagggaccacccctcagggcgcccccccacccccgcccggtccacctagacccccacgtttggagattcaaaacttctgtcttcgtcctctcccccgagccccctctcccaaatttttaaagcactttttagatttttttttctctttcctccttaaaaacaaaatttatatatagatatatatatatatataaataatatacttttcctcagaggagcaggcaacagtgtgggataaacagagtcacgatcagaggaaccccagggtctggtgatggcagggatggggggagagagagaaaatccacaaattccaatgtcacaaaagcaataaaacaaactagaaaaaaaaaaggttttacaaaatgaaaggaaggaaaaaaaaaaaggcaaccaaccacattagaagtcttggcactttgtaacggaacgggtactacactttatcttaattcttaatttaaaaacatgtttacaagttacaaccaacttctatgaaaagttgaaaagacaaaaaaaaaaaaaaaaagcgagcgagagagagagcgagagagagagcgagagcagaagaaattcctaaaagtcgatttatttttgtacaaaataataaaaaaaaaaacccaccacaaacgtagaatccacttctgttccccaaaaagcgagaaggggggttcaggaggaagccatcgcaggggacctgggagacgccccgaggtgtttgtgcttcacccccagacgtcagcctcgaaggcaggactgtggggtgttcgtgctgtgttccccccgctccccctttctgtccccttttttggttctgacgtgaagaggtcttagcgcccgcttctgtccacggggtctctccttcctcctccctagctcagggatgggccttccagccggagcaccccgatccccatccggcaccccccaatcccccaacacgcctgtccctcccgcatggccaccaaggagctggaccttggatgcgcctaccctgctgaggtgggtgacaggggccccccacctccagggccttagaaccaccgcccctctccccaccccaggcacccctctttttactcaaaggcactgactgtaatccagggggactgggacctgcctccccccaacctctggctcccacaaggcccggtgttgaccgagccacaggccacggacaggggccggggttggggagactatgtcgccagatgccaggacgccctcaccccgtttgcatatgcaatgctagcatgggaccccgaaaatagacgctctgctgcactgagacttcttgtcaatgcccaaccggcgggggggtgtctccctgcccccgacccccccatacccccttctctgtgacacacacatcttctcgtctctttttctttcattgttaaagggaagctttttaagaaggcaattttcatattgtttctacaggatggttttggttcccttcccttcccacccccccttaagcctgtcagccccctccaaatgtctcaggatcccccctctcccctggggctgggtgacagcaccccggctgcgttcacaccccagtgtcacagggcgagctgttctggagagaaaaccatctgtcgtggctgagcggggagcttgaacacccaggccagggacacccctccccagctcccagagaggccccctgaggggtgagccctctttccaccttcccctatccatgcaccccctcgcaataaaaccaactctaaaatcacagctgtcgtcctagccagtgggggcgaccggacttggggggtggagccctctgggacttccgtaggaacaagggctgcggcccaccgcgacacttacacagacctcggggattgcactaaaccctcgttcctagctccgcactcagcttcgcctgtcctgcccgcccactttgccttaactacccgcccgtcctgggggccacagcctctgcatgggcccagagccgggacccccccagcccagccccgccctccccagactccgcgcaatcacatactgtatatagacgtgaatcgattttatttttattctttaaattaaggtcgtgataaagtgttgccaaagatacctgctgaattctcgcgtttcaggaaacaaacaaacaaaaaaaaatgatatttgaggagggtcgtgttgactccatatgaaaggacacagctcaaagcttttttgtttggttgtttggggttttttgtgttttctttttttggggtgttttttttttaactgcctggtacaaaaaaaaaaagagaaaaaaaaaaaagaaaaacaatgcgaaattgttatttccattctcatggtgaagttgcgtggacgcgtgtgtgcgtgtgtgcaagagagcgggagtgaggtccaggctggggttggggggcttcaggcgggggcgcccgggggccggggaggtggccgggccggagcccccgtctgcagtgccccccagcctgccgggcccaggagagagagagaagcatctttgctactagctgttgctgctacctgcctctgccccccgacgccccccgccttttgagattaaggaaaaaaaaaaaaagtcaaaaaagtttttaaaaatgaaaaaaaaaaattataaaccagtgaatgtaaaatgccggagcaggcccggcctggcatgggtgtggacctgcagccaggcaggctcgagcgggcgataccaaagtctgcccccccaccattgtggccatgcagtcctgtcactgtctttttgcttccttccgaggggggtcccccagcctcttccagggtcttcccctggaagtgggcggctgcagggaaggtgggggacaggggtctttgcacgattcagaccccggggccgtggcaggagcggtcacctcacaggtggtgacactgaggcaggggcctcggggtgccccctcccgcccggcaaccagaatggttggaggcaagacagagagaatgaaaggaaaaacagaagaaaaaaaaatattaaaaaccaacaaaaaaagcaaaaatcctattttttgagaaagaaagatatttatatttgcagttttattttaaaaagttatttaagttgaagcagccttcctggaggtgggggggggggggtggtgggtggctggcgcaggacgggtcaggggcctggaggctgggggtgccccaggagctacaacctcagagttaagactagctcgcattaaatacatagatttacggggggggggggggggggccgggcccagggggtggagggggccagggagacccccatccctcgccggggctgcctggaggctgtggaccaggatccgatgcccaggtcccgccccccaccccaccccaggcccagaatcgaggtgccttggactttggaggggccaggcctggtgaatggggggcggggcggcgccctcagggtacagagcacagacagatagacattccagagactgtattgagagtctttataaagtgtgggagatttaaaaaaaaaaaaaactgataaaaatgcactttttgggagtggggagggagaagctttaaaagtaataaaaaacaaacaaaaacacaaaagatgaaaaaacaaaaaaattcatttttcttgtacataaaaaaaaaaaaagaaccactaaacgcagcctgttacgac

SEQ ID NO: 5 - protein, 584 aa, Homo sapiens
MAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQILAADAGADAGQLDLVDQIDQRNLLRAKEYLEFFQSNPMNSLPPAAAAAAASFPWSAFGASDDDLDATKEAVAAAVAAVAAGDCNGLDFYGPGPPAERPPTGDGDEGDSNPGLWPERDEDAPTGGLFPPPVAPPAATQNGHYGRGGEEEAASLSEAAPEPGDSPGFLSGAAEGEDGDGPDVDGLAASTLLQQMMSSVGRAGAAAGDSDEESRADDKGVMDYYLKYFSGAHDGDVYPAWSQKVEKKIRAKAFQKCPICEKVIQGAGKLPRHIRTHTGEKPYECNICKVRFTRQDKLKVHMRKHTGEKPYLCQQCGAAFAHNYDLKNHMRVHTGLRPYQCDSCCKTFVRSDHLHRHLKKDGCNGVPSRRGRKPRVRGGAPDPSPGATATPGAPAQPSSPDARRNGQEKHFKDEDEDEDVASPDGLGRLNVAGAGGGGDSGGGPGAATDGNFTAGLA

SEQ ID NO: 6 - DNA, 2451 bp, Homo sapiens
cgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttctttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccaaaaaaaaaaaaaaaaaaaa

SEQ ID NO: 7 - protein, 393 aa, Homo sapiens [sequence omitted]

SEQ ID NO: 8 - DNA, 2251 bp, Homo sapiens
gccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcggtctcttccagccctcagtctgaggttctgtattggaaagtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccagga

SEQ ID NO: 9 - protein, 641 aa, Homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSVSSSPQSEVLYWKVHGAHGDRRATVLASPLLASPLLASPLLASPVSAESTRSLQPALWHIPPPSLASPPAR

SEQ ID NO: 10 - DNA, 20 bp, Artificial Sequence
  Description of Artificial Sequence Synthetic primer
acaacgtccctctcggtaga

SEQ ID NO: 11 - DNA, 20 bp, Artificial Sequence
  Description of Artificial Sequence Synthetic primer
ccaagttatcagtcaccttc

SEQ ID NO: 12 - DNA, 23 bp, Artificial Sequence
  Description of Artificial Sequence Synthetic primer
gctccctcatgggcaaactcact

SEQ ID NO: 13 - DNA, 20 bp, Artificial Sequence
  Description of Artificial Sequence Synthetic primer
gggcagtgtggaaagaagag

SEQ ID NO: 14 - DNA, 22 bp, Artificial Sequence
  Description of Artificial Sequence Synthetic primer
cataacaggtgaggtgacgctg

SEQ ID NO: 15 - DNA, 23 bp, Artificial Sequence
  Description of Artificial Sequence Synthetic primer
tggctggtctacctggctcctct

SEQ ID NO: 16 - DNA, 20 bp, Artificial Sequence
  Description of Artificial Sequence Synthetic primer
cgtcgacaacggctccggca

SEQ ID NO: 17 - DNA, 20 bp, Artificial Sequence
  Description of Artificial Sequence Synthetic primer
caggatgttgacagcaagag

SEQ ID NO: 18 - DNA, 19 bp, Artificial Sequence
  Description of Artificial Sequence Synthetic primer
ggcctgctgttcacagttg

SEQ ID NO: 19 - DNA, 20 bp, Artificial Sequence
  Description of Artificial Sequence Synthetic primer
ctgcaaccaagtcttctcag

SEQ ID NO: 20 - DNA, 21 bp, Artificial Sequence
  Description of Artificial Sequence Synthetic primer
cttctgtgctccagggttctc

SEQ ID NO: 21 - DNA, 20 bp, Artificial Sequence
  Description of Artificial Sequence Synthetic primer
gctgctttgcctacctctcc

SEQ ID NO: 22 - DNA, 20 bp, Artificial Sequence
  Description of Artificial Sequence Synthetic primer
gctttcagcatccaagtgtg

SEQ ID NO: 23 - DNA, 21 bp, Artificial Sequence
  Description of Artificial Sequence Synthetic primer
gcctctcgtacatacagacgc

SEQ ID NO: 24 - DNA, 22 bp, Artificial Sequence
  Description of Artificial Sequence Synthetic primer
gtgtgtggcttttcaaacctca

SEQ ID NO: 25 - DNA, 20 bp, Artificial Sequence
  Description of Artificial Sequence Synthetic primer
actgcacccaaaccgaagtc

SEQ ID NO: 26 - DNA, 19 bp, Artificial Sequence
  Description of Artificial Sequence Synthetic primer
ccaaccaccaggctacagg

SEQ ID NO: 27 - DNA, 21 bp, Artificial Sequence
  Description of Artificial Sequence Synthetic primer
gattttgagaccatccagagc

SEQ ID NO: 28 - DNA, 20 bp, Artificial Sequence
  Description of Artificial Sequence Synthetic primer
tgcattccgcttagctttct

SEQ ID NO: 29 - DNA, 20 bp, Artificial Sequence
  Description of Artificial Sequence Synthetic primer
cacttcataacctccagatc

SEQ ID NO: 30 - DNA, 21 bp, Artificial Sequence
  Description of Artificial Sequence Synthetic primer
ggagttcgaggaaccctagtg

SEQ ID NO: 31 - DNA, 21 bp, Artificial Sequence
  Description of Artificial Sequence Synthetic primer
ccaagtgctgccgtcattttc

SEQ ID NO: 32 - DNA, 19 bp, Artificial Sequence
  Description of Artificial Sequence Synthetic primer
gtaaaccagtcagcctgag

SEQ ID NO: 33 - DNA, 20 bp, Artificial Sequence
  Description of Artificial Sequence Synthetic primer
ggaaatgaagccaaagtacc

SEQ ID NO: 34 - DNA, 19 bp, Artificial Sequence
  Description of Artificial Sequence Synthetic primer
caaggctggtccatgctcc

SEQ ID NO: 35 - DNA, 20 bp, Artificial Sequence
  Description of Artificial Sequence Synthetic primer
ggactgctttgagcgcaaag

SEQ ID NO: 36 - DNA, 19 bp, Artificial Sequence
  Description of Artificial Sequence Synthetic primer
aggtggctcttggaaggtg

SEQ ID NO: 37 - DNA, 22 bp, Artificial Sequence
  Description of Artificial Sequence Synthetic primer
gaaatgccaccttttgacagtg

SEQ ID NO: 38 - DNA, 21 bp, Artificial Sequence
  Description of Artificial Sequence Synthetic primer
gcacagttggcaacctttatg

SEQ ID NO: 39 - DNA, 22 bp, Artificial Sequence
  Description of Artificial Sequence Synthetic primer
aaatcaccatgccctctacaag

SEQ ID NO: 40 - DNA, 20 bp, Artificial Sequence
  Description of Artificial Sequence Synthetic primer
tttttccagcagaccagctt

SEQ ID NO: 41 - DNA, 20 bp, Artificial Sequence
  Description of Artificial Sequence Synthetic primer
ttctgtgctgtcccagtgag

SEQ ID NO: 42 - DNA, 20 bp, Artificial Sequence
  Description of Artificial Sequence Synthetic primer
atcgatttctcccctgtgaa

SEQ ID NO: 43 - DNA, 21 bp, Artificial Sequence
  Description of Artificial Sequence Synthetic primer
gtcggcttcttctccaatcag

SEQ ID NO: 44 - DNA, 22 bp, Artificial Sequence
  Description of Artificial Sequence Synthetic primer
tgggcctcgtcacccacatagg

SEQ ID NO: 45 - DNA, 19 bp, Artificial Sequence
  Description of Artificial Sequence Synthetic primer
catctttctgtaacactgg

SEQ ID NO: 46 - DNA, 21 bp, Artificial Sequence
  Description of Artificial Sequence Synthetic primer
ctgctggtgatcctcttgtag

SEQ ID NO: 47 - DNA, 20 bp, Artificial Sequence
  Description of Artificial Sequence Synthetic primer
gccggtttctcttagtcagg

SEQ ID NO: 48 - DNA, 21 bp, Artificial Sequence
  Description of Artificial Sequence Synthetic primer
ctgtctgcctcttttggtcag

SEQ ID NO: 49 - DNA, 21 bp, Artificial Sequence
  Description of Artificial Sequence Synthetic primer
tcgagtgacaaacacgactgc

SEQ ID NO: 50 - DNA, 20 bp, Artificial Sequence
  Description of Artificial Sequence Synthetic primer
gactactggtgatccttctg

SEQ ID NO: 51 - DNA, 21 bp, Artificial Sequence
  Description of Artificial Sequence Synthetic primer
ccagttctgctttggatcagc

SEQ ID NO: 52 - DNA, 22 bp, Artificial Sequence
  Description of Artificial Sequence Synthetic primer
tgcatgaactcactctttccag

SEQ ID NO: 53 - DNA, 22 bp, Artificial Sequence
  Description of Artificial Sequence Synthetic primer
tggggacaccttttagcatctt

SEQ ID NO: 54 - DNA, 19 bp, Artificial Sequence
  Description of Artificial Sequence Synthetic primer
gcgtcacactcaagctctg

SEQ ID NO: 55 - DNA, 21 bp, Artificial Sequence
  Description of Artificial Sequence Synthetic primer
ctcttcagtatcttcttgatg

SEQ ID NO: 56 - DNA, 20 bp, Artificial Sequence
  Description of Artificial Sequence Synthetic primer
cagaaggaggtctgtctgga

SEQ ID NO: 57 - DNA, 23 bp, Artificial Sequence
  Description of Artificial Sequence Synthetic primer
cacagtgaactcctggcctgtac

SEQ ID NO: 58 - DNA, 21 bp, Artificial Sequence
  Description of Artificial Sequence Synthetic primer
gggatttgtagtggatcgtgc

SEQ ID NO: 59 - DNA, 21 bp, Artificial Sequence
  Description of Artificial Sequence Synthetic primer
ggctcgcagggatgatttcaa

SEQ ID NO: 60 - DNA, 20 bp, Artificial Sequence
  Description of Artificial Sequence Synthetic primer
gctttctccaggtactcttg

SEQ ID NO: 61 - DNA, 21 bp, Artificial Sequence
  Description of Artificial Sequence Synthetic primer
gatgaagcgtttggtgctctg

SEQ ID NO: 62 - DNA, 23 bp, Artificial Sequence
  Description of Artificial Sequence Synthetic primer
tgctatcacttcctttctgttgc

SEQ ID NO: 63 - DNA, 20 bp, Artificial Sequence
  Description of Artificial Sequence Synthetic primer
ctgagtgctctgactatgtg

SEQ ID NO: 64 - DNA, 23 bp, Artificial Sequence
  Description of Artificial Sequence Synthetic primer
ggtgacatcgtttgagaaattgc

SEQ ID NO: 65 - DNA, 21 bp, Artificial Sequence
  Description of Artificial Sequence Synthetic primer
tggatgctctcatcaggacag

SEQ ID NO: 66 - DNA, 21 bp, Artificial Sequence
  Description of Artificial Sequence Synthetic primer
tgattgtcctggtttgtgtcc

SEQ ID NO: 67 - DNA, 22 bp, Artificial Sequence
  Description of Artificial Sequence Synthetic primer
cccacttttatcaccatcgcaa

SEQ ID NO: 68 - DNA, 20 bp, Artificial Sequence
  Description of Artificial Sequence Synthetic primer
agagattatcggagcgcctt

SEQ ID NO: 69 - DNA, 20 bp, Artificial Sequence
  Description of Artificial Sequence Synthetic primer
tgaagaaaaccccttgtgct

SEQ ID NO: 70 - DNA, 21 bp, Artificial Sequence
  Description of Artificial Sequence Synthetic primer
tgtcaaattcattcatggcct

SEQ ID NO: 71 - DNA, 21 bp, Artificial Sequence
  Description of Artificial Sequence Synthetic primer
catcacgacaggaatgaccag

SEQ ID NO: 72 - DNA, 19 bp, Artificial Sequence
  Description of Artificial Sequence Synthetic primer
tggcacccagcacaatgaa

SEQ ID NO: 73 - DNA, 21 bp, Artificial Sequence
  Description of Artificial Sequence Synthetic primer
ctgttggtgctgctgctgctg

SEQ ID NO: 74 - DNA, 21 bp, Artificial Sequence
  Description of Artificial Sequence Synthetic primer
tgctgccactaatgctgatgt

SEQ ID NO: 75 - DNA, 20 bp, Artificial Sequence
  Description of Artificial Sequence Synthetic primer
gacctgtcactgtcttgtac

SEQ ID NO: 76 - DNA, 21 bp, Artificial Sequence
  Description of Artificial Sequence Synthetic primer
ctaagtcatagtccgcctaga

SEQ ID NO: 77 - DNA, 20 bp, Artificial Sequence
  Description of Artificial Sequence Synthetic primer
cgaacacttgcagattactg

SEQ ID NO: 78 - DNA, 22 bp, Artificial Sequence
  Description of Artificial Sequence Synthetic primer
ctcaggaaccaatctttgcact

SEQ ID NO: 79 - DNA, 21 bp, Artificial Sequence
  Description of Artificial Sequence Synthetic primer
cttcctcttggagaagatcag